(19) United States
(12) Patent Application Publication
(10) Pub. No.: US 2013/0184693 A1
(43) Pub. Date: Jul. 18, 2013

(54) DEVICE AND METHOD FOR TREATING MEDICAL, SKIN, AND HAIR DISORDERS WITH ENERGY

(71) Applicant: Joseph Neev, Laguna Beach, CA (US)

(72) Inventor: Joseph Neev, Laguna Beach, CA (US)

(21) Appl. No.: 13/713,267

(22) Filed: Dec. 13, 2012

Related U.S. Application Data
(63) Continuation-in-part of application No. 13/681,393, filed on Nov. 19, 2012.
Application No. 13/681,393 is a continuation-in-part of application No. 11/356,760, filed on Feb. 17, 2006, now abandoned.
(60) Provisional application No. 61/594,969, filed on Feb. 3, 2012.

(51) Int. Cl. A61B 18/18

(52) U.S. Cl. CPC A61B 18/18
USPC 606/9; 606/10

(57) ABSTRACT

A device and a method for thermal treatments of target material with various thermal interactions are disclosed. A device for treating hair on the skin comprises a treatment head coupled to a housing; a hair remover for removing the hair from a target area of the skin; a light source for transmitting a predetermined amount of energy to the skin. A device and method for treatment of tissue. The device comprises of an energy source for treatment of surface and subsurface tissue, And of a mechanical source of energy for mechanically deforming the treated tissue. Both the Mechanical Energy (ME) and the Treatment Energy (TE) may be either continuously operating (CO) or modulated in time and space.

### CROSS-REFERENCE TO RELATED APPLICATIONS

This application is a continuation-in-part of U.S. patent application Ser. No. 13/681,393, filed Nov. 19, 2012, and this application is a continuation-in-part of U.S. patent application Ser. No. 11/356,760, filed Feb. 17, 2006, which claims the priority benefit of U.S. Provisional Application Ser. No. 60/653,826 filed on Feb. 17, 2005 and U.S. Provisional Application Ser. No. 60/668,678 filed on Apr. 6, 2005, and this application claims priority to U.S. Provisional Application Ser. No. 61/594,969 filed on Feb. 3, 2012, all of which are hereby incorporated by reference in their entirety.

### BACKGROUND

The present invention relates to the fields of the treatment and conditioning of skin, hair, pain, or a medical condition.

Skin disorders, such as acne, can be irritating and embarrassing. The major disease of skin associated with sebaceous follicles is acne vulgaris. This is also the most common reason for visiting a dermatologist in the United States. There are many treatments, but no cures for acne. These include antibiotics (which inhibit growth of p. acnes bacteria which play a role in acne), retinoids such as Accutane™ (isotetinoin, which reduces sebaceous gland output of sebum), and antimicrobials such as benzoyl peroxide.

Acne lesions result from the rupture of a sebaceous follicle, followed by inflammation and pus (a “whitehead”), or by accumulation of plugged material in the sebaceous follicle (a “blackhead”). This condition has two major requirements: (1) plugging of the upper portion of the follicle, and (2) an increase in sebum production. The upper portion of the follicle, i.e., the “pore” into which sebum is secreted and which is directly in contact with the skin surface, is called the infundibulum. A plug forms in the infundibulum from cells, sebum, bacteria, and other debris. The sebaceous gland continues to produce sebum (an oily fluid), stretching the infundibulum until either it or some lower portion of the follicles ruptures.

In most males, acne is worst in the teenage years and then subsides, in women, teenage acne is often followed by menstrual acne flares well into adulthood. It is well known in the art that both plugging of the infundibulum and high sebaceous gland activity are necessary for an acne lesion to develop. Several methods known in the art are aimed at reducing gland activity or inhibiting bacteria. The drug Acutane is approved by the FDA but is taken orally and has severe side effects such as skin dryness, birth defects and severe depression. Light based method in conjunction with cooling are used to at least partially disable the sebaceous glands. These methods too result in skin dryness due to the damage cause to the sebaceous glands and usually require high energy level which are potentially hazardous and require doctor-only operation. As the consequence of the relative invasiveness of the procedure, interaction with live tissue, and high laser power level needed, the instrument are relatively expensive. Both methods require time to take effect and results are generally monitored over period of weeks and months.

Methods for removing unwanted hair include shaving, waxing, plucking unwanted hair as well as technology driven methods. Such technologies include electrolysis wherein a needle is inserted to each unwanted hair shaft and the hair is removed.

Excess hair and/or unwanted hair are common dermatological and cosmetic problems, and can be caused due to heredity factors, illness and diseases, hormonal activity or other factors. Hair can be temporarily removed using a number of techniques such as shaving, wax epilation, depilatory creams, or more permanently removed using electrolysis. Electrolysis is a process that involves insertion of a current-carrying needle into each hair follicle, and is often painful, inefficient, and time consuming. In addition, high power light and laser based methods also allow reduction of hair growth. However, laser and high power light based technologies can result in significant damage to the patient skin or skin components, and thus require operation by a physician or dermatologist. These treatments are often expensive and inefficient.

A need therefore exists for a device that allows hair conditioning and hair growth reduction for a limited time periods. Such devices should be relatively inexpensive to be affordable to large numbers of consumers and should be designed for use by non-medical personnel and wide use at home and in barber and beauty shops. Such a device does not exist at this time.

It is known in the art to use massage to treat body tissue or muscle. Massage is the working of superficial and deeper layers of muscle and connective tissue using various techniques, to enhance function, aid in the healing process, and promote relaxation and well-being.

Massage involves working and acting on the body with pressure—structured, unstructured, stationary, or moving—tension, motion, or vibration, done manually or with mechanical aids. Target tissues may include muscles, tendons, ligaments, fascia, skin, joints, or other connective tissue, as well as lymphatic vessels, or organs of the gastrointestinal system. Massage can be applied with the hands, fingers, elbows, knees, forearm, and feet. There are over eighty different recognized massage modalities. The most common reasons for introducing massage as therapy have been client demand and the fact that many recipient of such a treatment feel clinical effectiveness and improvement.

In professional settings massage involves the client being treated while lying on a massage table, sitting in a massage chair, or lying on a mat on the floor. The massage subject may be fully or partly unclothed. Parts of the body may be covered with towels or sheets. Those who practice massage as a career are referred to as massage therapists. Most states in the US have licensing requirements for massage therapists.

Percussive massage involves repeating the application of mechanical impact to the body tissue and muscle. Deep tissue massage is designed to relieve severe tension in the muscle and the connective tissue or fascia. This type of massage focuses on the muscles located below the surface of the top muscles. Deep tissue massage is often recommended for individuals who experience consistent pain, are involved in heavy physical activity (such as athletes), and patients who have sustained physical injury. It is not uncommon for receivers of deep tissue massage to have their pain replaced with a new muscle ache for a day or two. Deep tissue work varies greatly.

The term “deep tissue” is often misused to identify a massage that is performed with sustained deep pressure. Deep tissue massage is a separate category of massage therapy, used to treat particular muscular-skeletal disorders and complaints and employs a dedicated set of techniques and strokes to achieve a measure of relief. It should not be confused with “deep pressure” massage, which is one that is performed with sustained strong, occasionally intense pressure throughout an entire full-body session, and that is not performed to address a specific complaint. Deep tissue massage is applied to both the superficial and deep layers of muscles, fascia, and other structures. The sessions are often quite intense as a result of the deliberate, focused work. When a client asks for a massage and uses the term “deep tissue”, more often than not he or she is seeking to receive a full-body session with sustained deep pressure throughout. If a practitioner employs deep tissue techniques on the entire body in one session, it would be next to impossible to perform; it might lead to injury or localized muscle and nerve trauma, thereby rendering the session counterproductive.

For treatment of muscle soreness the increased blood flow to the muscle, for example with the application of low-intensity work, massage, hot baths, or a sauna visit may help somewhat. Immersion in cool or icy water, an occasionally recommended remedy, was found to be ineffective in alleviating muscle soreness. Additionally, deep muscle massage increases the flow of fresh blood into the treated regions. It can also break up adhesion and scar tissue, improves blood circulation, help remove lactic acid deposits, and restore muscle flexibility.

Light therapy and, in particular, the application of Near Infrared (NIR) light have been shown to increasing localized blood circulation (micro-circulation) and the reduction of pain. For example, NIR therapy can comprise application of laser light, broad band light with NIR transmitting fillers, or Light Emitting Diodes (LED) in the NIR region of the EM spectrum. For example, High-power infrared LEDs allow for effective, deep penetration into soft tissue, which induce biological effects that take place.

The present invention attempts to solve these problems as well as others.

### SUMMARY OF THE INVENTION

It is therefore useful to have a method and a device that is relatively low cost and effective in treating active acne condition. Such a method and a device includes a low power light or electromagnetic energy source or a source of electric power. The energy source is used to rapidly generate thermal energy deposition in the upper layers of the skin which then result in opening and drainage of the pores. The enlarging of the pores then results in drainage of the sebum and any other liquid or debris trapped within the pores, and with them, the acne causing bacteria or any other infectious or diseases causing components.

In particular, such devices can be hand held and constructed of low power photographic light bulb such as the ones used in single-use or small digital cameras. Other energy sources can be heating elements including electrical resistors that can generate high temperatures by use of a current or an electric heater. Such energy source can be powered by low cost transformers or batteries or electric line, be controlled by small electronic board and discharge their energy from a storage capacitor at variable discharge pulse durations. Such an assembly can be very inexpensive and as result yield low cost home or consumer use device or low cost, cosmeticians, aestheticians or physician use devices.

Because energy is delivered to the uppermost layers of the skin only to allow opening of the pores, the method and the devices are very safe. (energy diffusing below the epidermal dermal junction) are not high enough to cause collateral damage.) Because the expansion is very rapid and the drainage of the pore begins immediately, the response of the acne is very rapid and results can be observed from as little as a few hours or less.

The method utilizes the principle of application of thermal energy to the upper section of the skin such that the skin upper layers are forced to expand (fully or partially) in a manner that results in temporary expansion of the pores and pore openings, thereby treating skin disorder. The method and devices envision thermal energy delivered directly from a source, via the mediation of a heating element capable of depositing such expansion-causing thermal energy on the surface of the skin. One embodiment envisions light or electromagnetic (EM) energy as the energy source for the expansion causing energy. In particular one embodiment envisions the use of low cost flash lamp of the kind used in disposable or digital (or single-use) cameras, to deposit such thermal energy in the skin. This embodiment further envisions the possibility of use of an absorbing intermediate substance which can partially or fully absorb the EM energy to create thermal energy deposition on the surface of the skin.

The use of such low power light source significantly reduces the cost of the systems, their size, and thus make such treatment devices useful for home and consumer use. The use of a system which to a large extent use components of disposable, single use, or consumer digital camera, also increases safety level in a significant way (people expose themselves and others multimillion times a day to such energy level while taking photographs), and thus reduces both the risk of collateral damage and unwanted damage and risk to tissue and human.

Electrical energy to heat tissue and treat skin conditions can also be used. Here however, there is a risk of over-heating, as in all case of application of energy to tissue, but in addition, there is a risk of electric shock and electrocution. To mitigate these risks, the use of a transport of heat from an electrical heat source to the target tissue is applied as well as other components to limit the amount of electric energy and heat deposited in the tissue.

A heat shuttle is “loaded up with thermal energy” and then delivers its thermal energy to the skin in lump quanta of thermal energy. The use of an electro-optic system such as a laser, or a flash lamp with a topically applied high absorbing substance or a film capable of absorption of such optical energy.

More specifically, the method and apparatus described herein are also applicable for treating skin conditions and skin ailments and in particular, acne conditions. Acne lesions result from the rupture of a sebaceous follicle, followed by inflammation and pus (a “whitehead”), or by accumulation of plugged material in the sebaceous follicle (a “blackhead”). The creation of this condition requires two elements: (1) plugging of the upper portion of the follicle, and (2) an increase in sebum production. The upper portion of the follicle, i.e., the “pore” into which sebum is secreted and which is directly in contact with the skin surface, is called the infundibulum. A plug forms in the infundibulum from cells, sebum, bacteria, and other debris. The sebaceous gland continues to produce sebum (an oily fluid), stretching the infundibulum until either it or some lower portion of the follicles ruptures. The method and apparatus described herein, allows the skin upper layers to temporary expand under the influence of energy deposited into this target region thus allowing treatment of the skin disordered, and in particular, acne.

The use of several energy sources to achieve the acne and skin treatment effects is applied and includes: optical energy, optothermal conversion of optical energy to thermal—tissue expanding energy, electrical energy and electro-thermal conversion of electrical energy to thermal energy and mechanical energy source. An electrical energy source may also heat up an intermediate material that is then brought into contact with the tissue surface to achieve treatment and expansion. The electrical heated intermediate material may be disconnected from the heat source and then brought into contact into with the targeted tissue. Alternatively the heater source may remain connected to the electrical source and the electrical source discharge and deliver its energy to the tissue after said energy is converted to thermal energy in the device.

A device and method for reducing hair growth provides a hand held device that can be used safely to affect a portion of the hair shaft and follicular structure to allow reduction of hair growth. In one embodiment, the hand held light or energy emitting device with an on/off switch and a button that pulses the device light output when it is placed on the target site. A battery within the device or an external electrical power source powers a circuit board and drives a short pulse of lights. The light emitting devices has sufficient energy to cause at least some reduction in hair growth.

In another embodiment, light is emitted from the device and impinges on a portion of the skin that has been treated with at least some light absorbing substance. The substance is applied to the skin prior to the application of light and has been at least partially retained in at least some of the hair follicle containing pores in the skin.

In yet another embodiment, light is emitted from the device and impinges on a portion of the skin that has been treated with at least thermally conducting substance that allows heat to flow from the absorbing follicle shaft along the length of the follicle towards the hair papilla.

The amount of energy impinges on the skin is limited so that no permanent damage is caused to any portion of the skin. In general, such light emitting devices would generate fluence on the target skin in the range of from about 0.1 J/cm2 to as much as 20 J/cm2 and preferably from about 0.5 J/cm2 to 7 J/cm2.

In another embodiment, the light emitted by the light source has a broad band radiation ranging from about 300 nm to about 1400 nm and preferably from about 350 nm to 5 about 1200 nm. The pulse duration of the light is form 0.001 ms to about 1000 seconds and, preferably, from about 0.01 ms to about 500 ms.

In yet another embodiment, the device for treating hair utilizes a combination of two energy sources; the energy source emission of treating energy is synchronized so that the first prep source prepares the hair follicle for the action by the 10 subsequent energy sources which permanently modify its hair growing characteristics.

In yet another embodiment, the device for treating hair incorporates a safety component in the form of protruding guards that prevent energy from being emitted unless the device is firmly secured and in tight contact with a HEALTHY Part of the skin. This safety feature prevents the energy from the device from causing damage to the skin or to the eye.

The proposed combination is the mechanical tissue impact and tissue massage, with the application of Electromagnetic (EM) energy or light energy application. The treatment combines the benefits of both treatment regime and, additionally and importantly, enhance the effectiveness of each treatment by the simultaneous or time-adjacent (i.e. before, during or after) the application of the other

For example, application of mechanical massage or mechanical impact on the targeted tissue, before, during, or after (or a combination of such different timing, e.g. both before and during, or e.g. before, and after, or e.g. before, during and after, etc.), may enhance the penetration and delivery of light into the targeted tissue or targeted muscle.

Similarly, the application of light, for example, before, during or after the application of mechanical energy, may warm up the tissue or targeted muscles, and enhance circulation, which will make the massage action or mechanical impact more effective, and the targeted tissue, warmer, softer, and able to absorb the mechanical impact more easily.

Other objects and advantages of the present invention will become apparent from the following description and accompanying drawings.

### BRIEF DESCRIPTION OF THE DRAWINGS

In the accompanying figures, like elements are identified by like reference numerals among the embodiments.

FIG. 1a shows a sectional view taken through the handheld acne treatment device that uses a light energy sources and high absorbing intermediate layer to deliver energy into the skin.

FIG. 1b is a schematic diagram of a device suitable for practicing the methods to treat hair on a portion of a human skin.

FIG. 1c shows is schematic representation of another embodiment of the device and method for treatment of targeted tissue.

FIG. 1d is a schematic representation of the device and method for treatment of targeted tissue.

FIG. 1e is a schematic representation of the treatment head used in the device and method for treatment of targeted tissue.

FIG. 2a shows an exemplary circuit diagram for pulsing the energy source (light, electrical, or mechanical discharge, or preferably, a flash lamp)

FIG. 2b is a schematic diagram of a device suitable for practicing another version of the methods to treat human hair and its position against an exemplary portion of a facial skin.

FIG. 2c shows is schematic representation of another embodiment of the device and method for treatment of targeted tissue with mechanical energy transfer.

FIG. 3a shows a sectional view taken through the Treatment Head of a hand-held acne and skin treatment device, including light energy windows with optional light absorbing intermediate substance, electrical heater treatment window, and other forms of energy sources.

FIG. 3b is a schematic diagram of the elements of a device suitable for practicing a version of the methods and its relevant composition.

FIG. 3c is a schematic representation of a treatment head design

FIG. 3d is a schematic representation of additional exemplary embodiments of a treatment head design.

FIG. 3e is a schematic representation of additional exemplary embodiment possible for a treatment head design.

FIG. 4a shows a sectional view taken through another embodiment of the treatment head and high absorbing intermediate substance composition.

FIG. 4b shows a schematic representation of an exemplary mechanical impact generation.

FIG. 4c shows an additional exemplary embodiment possible for a treatment head design, including embedded treatment energy sources.

FIG. 5a shows a sectional view taken through the handheld acne treatment device that utilizes flash lamps as an energy source, reflectors, and an optical absorber to deliver energy into the skin. Multi-lamp system is shown.

FIG. 5b is a schematic diagram of the elements of another exemplary circuitry suitable for driving and controlling a flash lamp light source.

FIG. 5c shows an exemplary, non-limiting, block diagram for a treatment device.

FIG. 6a shows an exploded view taken through an embodiment illustrating one embodiment of a handheld acne treatment device with flash lamps light source, electrical transformer source, and a high absorbing intermediate layer.

FIGS. 6b-6d are a schematic representation of the steps taken in practicing one of the methods for treating hair.

FIG. 6e shows a schematic representation of an exemplary device for a treatment and removal of tattoos and other unwanted targets within a tissue or targeted material.

FIG. 7a shows a sectional schematic view taken through another embodiment of the hand-held acne treating device.

FIG. 7b is a schematic diagram of a device suitable for practicing the two energy source.

FIG. 8 shows a sectional view taken through another embodiment of treatment head showing both side view and a view from the bottom.

FIG. 9 shows another embodiment of the composition and structure of the high absorbing intermediary layer in the handheld acne treatment device.

FIG. 10 shows a treatment head of a handheld acne treatment device including multiple-treatment heads of both light with high absorbing intermediate layer as well as optical energy alone.

FIG. 11 illustrates two other possible treatment heads configuration utilizing a variety of multiple treatment windows that can be move and replaced within a treatment.

FIG. 12 illustrates how the handheld acne treatment device might be used on the skin of a human face

FIG. 13 is a sectional view showing the component of the enclosures of a possible embodiment of the handheld acne treatment device.

FIG. 14 is a sectional view showing a possible circuit driving an electrical discharge to generate an electric pulse heating of the hand held acne treatment device.

FIG. 15 is a sectional view showing the components of a light or electromagnetic radiation handheld acne treatment device with possible energy absorbing intermediate layer.

FIG. 16 is a sectional view showing the components of the handheld acne treatment device with a dispenser to allow the delivery of drug, nutrient or other elements to the skin.

FIG. 17 is a sectional view showing the components of the enclosures of a possible embodiment of a skin treatment device utilizing a light source and a high absorbing substance being rolled up and replenished by the motion of two rollers.

FIG. 18 is a sectional view showing the components of an electrical heating delivering its energy to the skin through the intermediate use of a movable heat carrier.

FIG. 19 is a sectional view showing the components of an electrical heating delivering its energy to the skin through the intermediate use of a movable heat carrier further comprising the use of a shutter.

FIG. 20 is a sectional view showing the component of a light based handheld acne treatment device utilizing motor-driven mirror scanning and shutter operation.

FIG. 21 is a sectional view showing the components of the enclosures of a possible embodiment of a skin treatment device utilizing a light source and a high absorbing substance of various pattern and various degrees of transmission being rolled up and replenished by the motion of two rollers.

FIG. 22 is a sectional view showing the component of an electric heating transport heat shuttle with disposable shuttle units.

FIG. 23 is a sectional view showing possible components of a heat transporter heat shuttle.

FIG. 24 is a sectional view showing possible components of an optical light or flash lamps handheld acne treatment device.

FIG. 25 is a sectional view showing possible components of an electrical heat handheld acne treatment device.

FIG. 26 is a sectional view showing possible components of an electrical and light thermal energy generator handheld acne treatment device.

FIG. 27 is sectional view showing another possible component configuration of an electrical and light thermal energy generator handheld acne treatment device.

FIG. 28 is a sectional view showing possible components of a light or flash-lamp, or electromagnetic energy source handheld acne treatment device with a removable element of high absorbing substance.

FIG. 29 is a sectional view showing possible components of an optical light, flash lamps, electric heater, or mechanical treatment switchable treatment windows.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

A device and a method for treating skin illnesses and improving skin condition and appearance is disclosed. Making use of thermal energy, the device treats skin conditions such as wrinkles, fine lines, skin lesions, cysts, warts, and improving the appearance of the skin. The device is a low cost, safe hand held device for treating the outer layer of the tissue and skin without undesirable injuries to the skin.

One embodiment aims at treating skin conditions and enhancing the appearance of the skin by depositing sufficient amount of energy into the skin surface to mitigate skin ailments and also allows external products to be able to better penetrate the skin surface thus enhancing skin conditions and healing the skin. Further, the device aims at doing the above by minimizing collateral damage to the skin.

One embodiment also contemplates accomplishing many of the above tasks by using a low cost hand held devices. Such a device is designed to utilize inexpensive components and is often powered by batteries or transformers. It limits the amount of energy deposited to the surface of the skin, resulting in relatively large concentration of energy at the upper surface of the skin so that beneficial physical effects are created healing the skin and improving its condition, but the amount of energy that the device deposit into the skin is not large enough to create collateral or unwanted damage to the living tissue of the skin.

In one embodiment, the device utilizes the low cost components of disposable, single use, or digital cameras to generate light energy, converting it to heat, and thus healing the skin and improving skin conditions.

Another embodiment is a device for controlling hair growth comprises: an optical element with variable power levels, at least one light source, a circuit to deliver a fixed amount of energy to the light sources, means to activate and trigger the circuit.

In a further embodiment, the device above can be used wherein the circuit to deliver a fixed amount of energy to the light source also allows the user to adjust the light source power level such that no permanent damage or alteration occur to any living tissue in the target skin.

An alternative embodiment can include a device for reducing the presence of hair on the skin, the device comprising: a handheld compact light source, a circuit to deliver a predetermined amount of energy to the light source, and a trigger to activate and trigger the circuit. More specifically, the light source can be a flash lamp, in particularly the type of flash lamps used in single use cameras. Alternatively the device can use a light source such as LED, lasers, lamps, tungsten lamps or other electromagnetic light sources.

In another embodiment, the device comprises an enclosure, said enclosure contain a Treatment ENERGY source (TES), a controller, and a MECHANICAL energy source (MES). The energy from the Mechanical Energy source is transferred to a Treatment Head through a Mechanical Energy Transfer mechanism. The energy from the Treatment Energy source is transferred to a Treatment Head through a Treatment Energy Transfer mechanism. The treatment Head may comprises a mass capable of delivering mechanical Impact to the treatment area or targeted media.

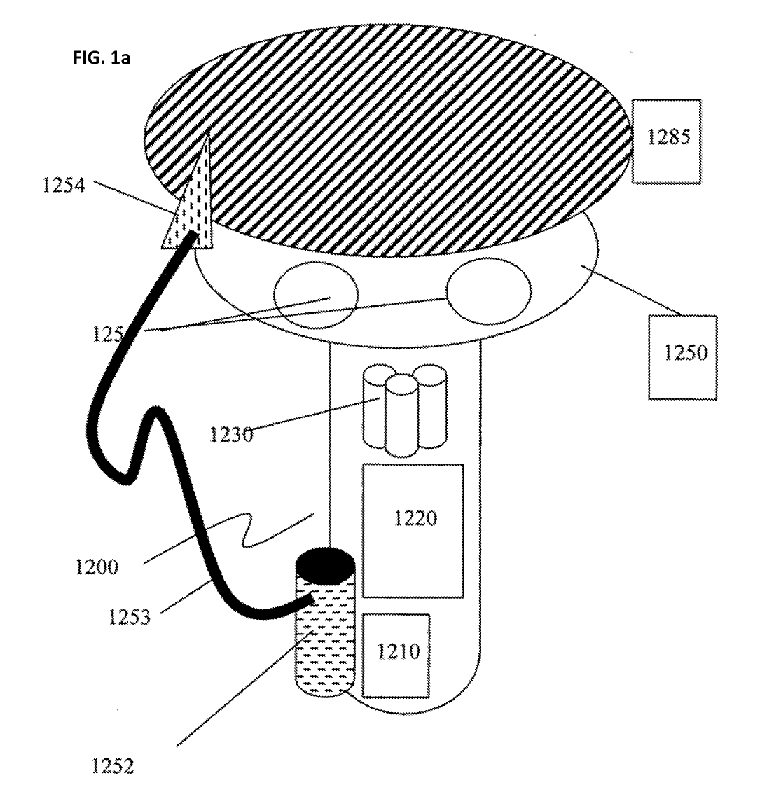

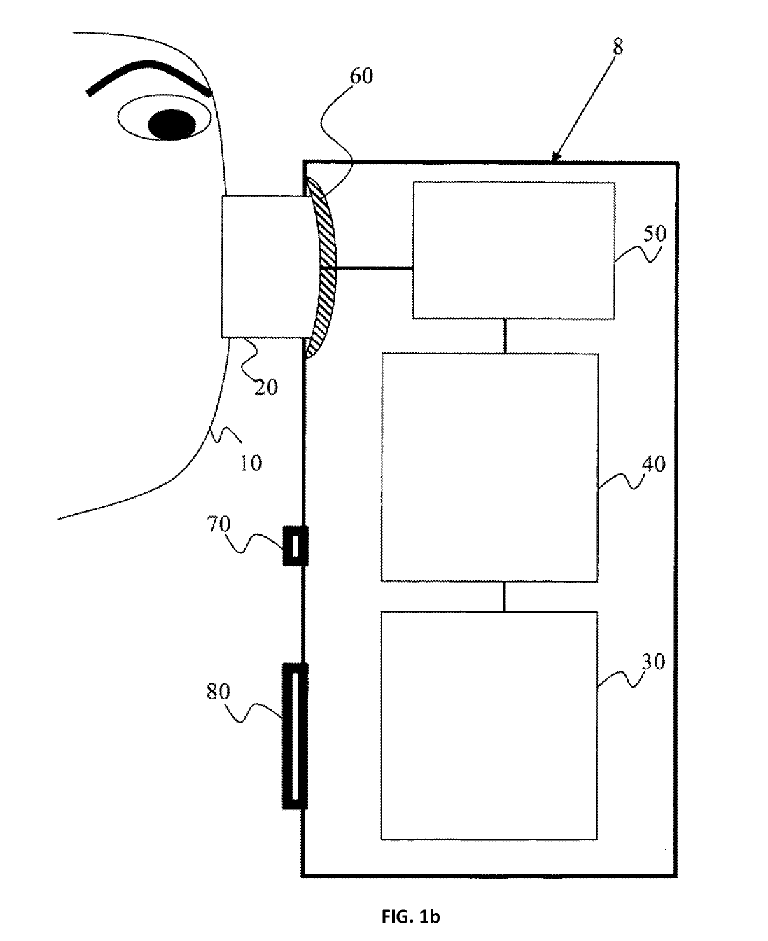

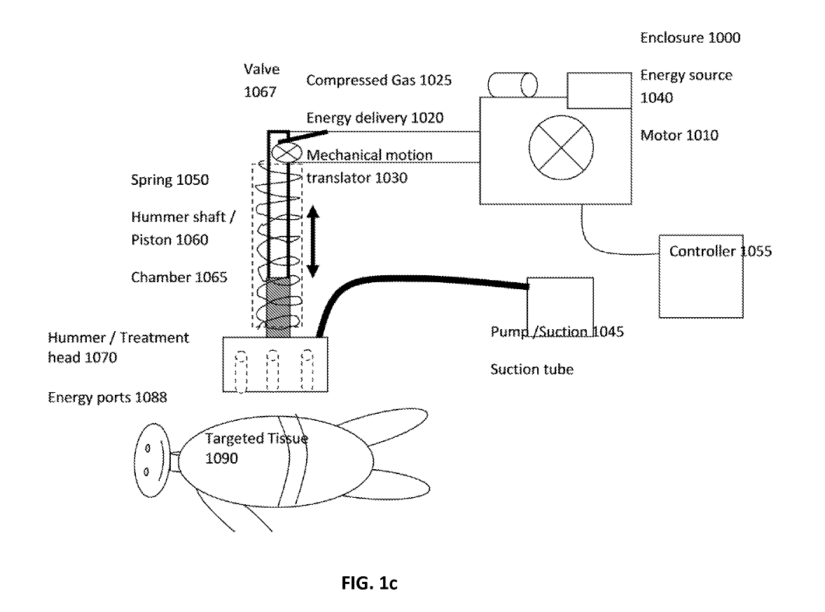

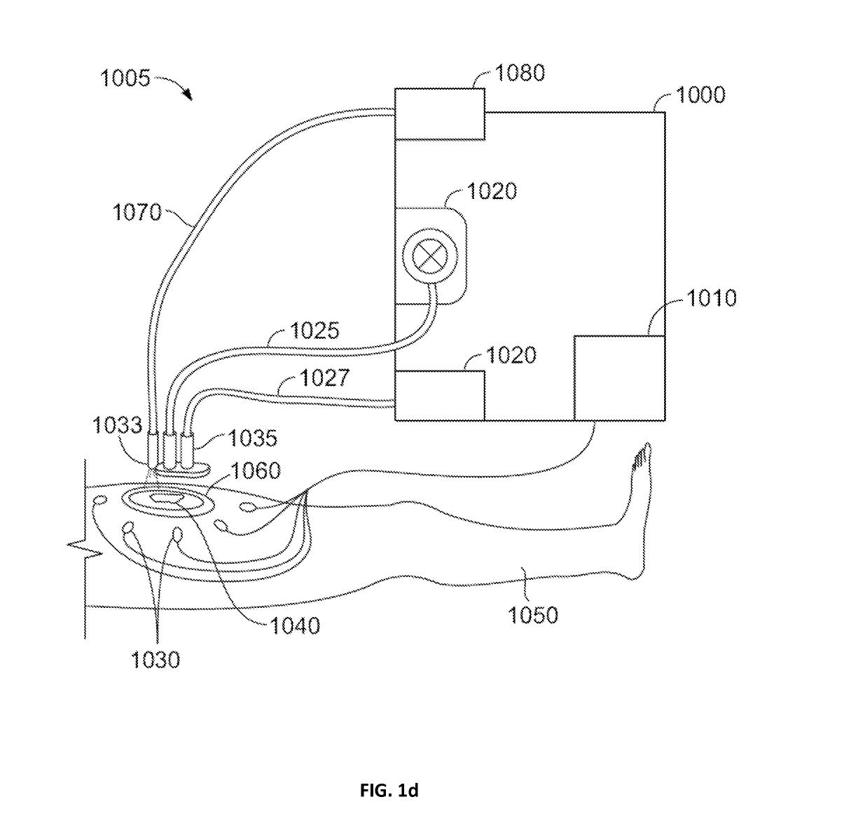

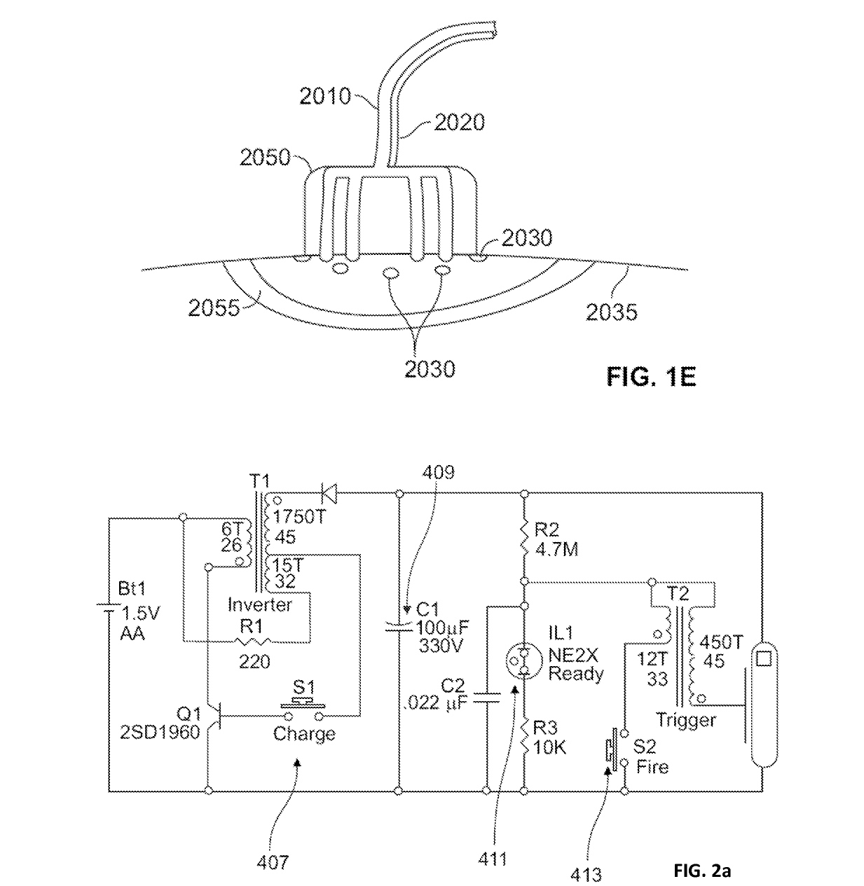

In an embodiment shown in FIG. 1a, a device comprises a casing 1200 that contains a power source 1210 (a battery or a wall-plug transformer, or a power supply or a power cord), a controlling circuit 1220, plurality of electric energy storage capacitors 1230, wires and connections to the treatment head 1250 where a plurality of treatment windows 1255 may be incorporated. FIG. 1a also shows a layer of absorbing substance 1285 contemplated for use in one embodiment for conversion of the treatment source energy into thermal energy.

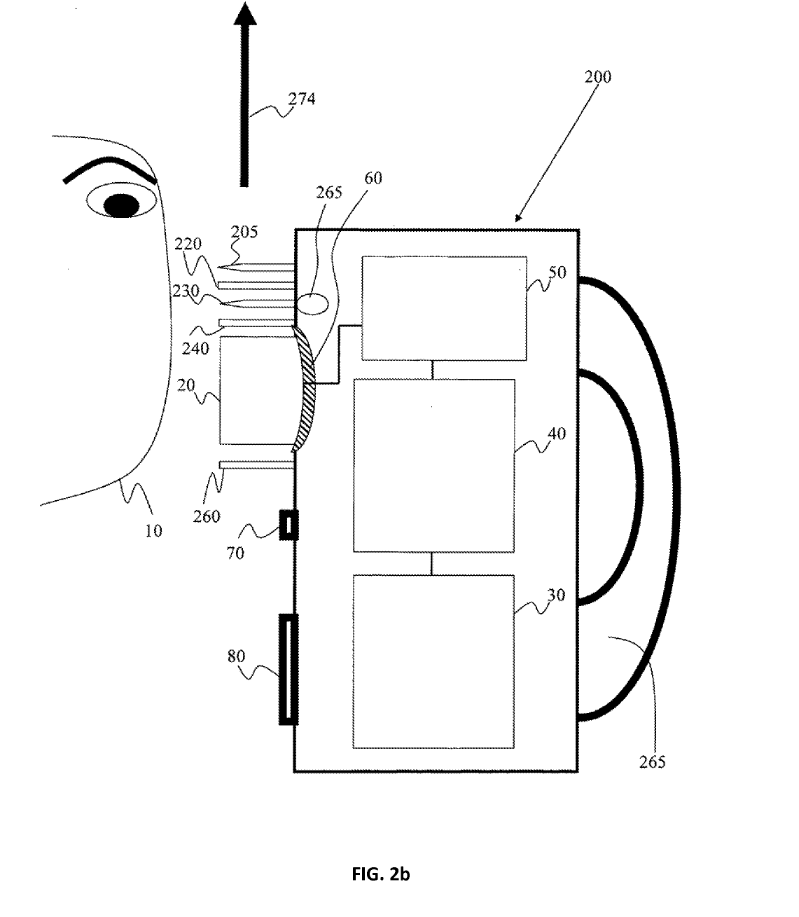

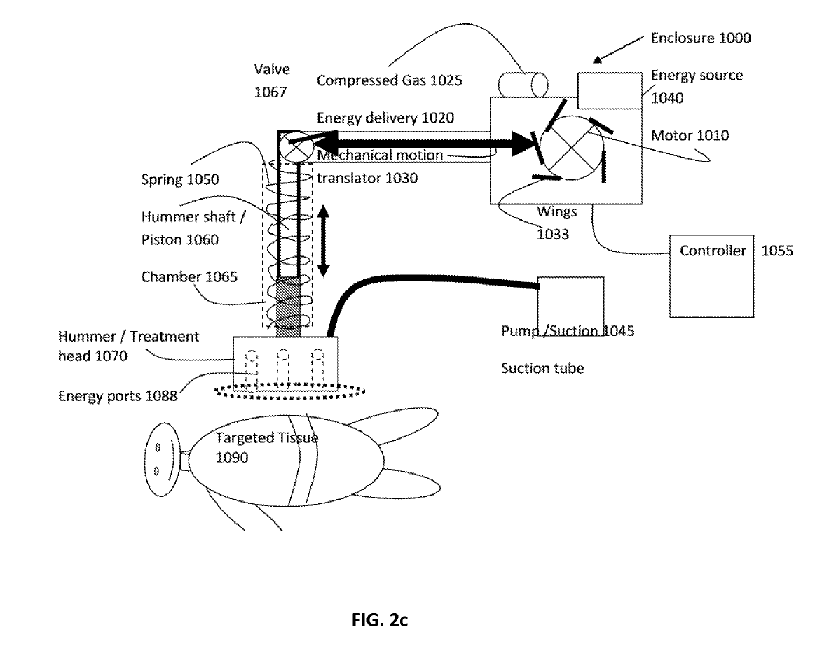

In another embodiment, the device is aimed at treating acne. In this embodiment, the device utilizes a small flash lamp such as the Perkin Elmer CGD 0013 or Perkin Elmer CGAC 2018 or Perkin Elmer BGAC 3022. Such flash lamps can be powered and controlled by an electronic board of the type shown in FIG. 2a. FIG. 2a illustrates an exemplary circuit 400 for driving an optical discharge hand held treatment device. A switch 407 is turned on to activate the device and charge the capacitor 409. When the capacitor is fully charged a lamp 411 (or LED) turns on and the circuit is ready to fire. Push button 413 is pressed to trigger the flash lamp which discharges capacitor 409. After firing, the capacitor 409 again begins to charge and after several seconds (depending on battery and resistance) is fully charged. This circuit releases a maximum energy per pulse of ½ CV², where C is the capacitor's capacitance and V is the final voltage across the capacitor. By selecting appropriate values of C, and V the released energy can be kept below the threshold for tissue burns. The embodiments disclosed herein can use any suitable conventional circuit for the above firing process.

FIG. 1b shows the simplest configuration envisioned for a device 8 according to another embodiment. After shaving the skin or removing the hair of the surface of the skin 10 by conventional means such as shaving, waxing or plucking, the skin is washed, cleaned and dried. The area targeted for treatment is then exposed to a flash lamp 20 or other suitable light source. The flash lamp 20 can be, for example, of the same type used in disposable cameras and digital camera flash. The flash lamp 20 is powered by one or more batteries 30 and one or more capacitors 40, and its operation is controlled by a small circuit board 50. The batteries 30, capacitors 40 and circuit board 50 can be, for example, similar to those used in a digital camera or in disposable cameras with flash such as the Kodak disposable extended range camera.

Alternatively, the capacitors can also be powered by direct contact to 110V or 220V power line from a domestic wall outlet of a domestic or industrial line. A switch 70 allows charging and firing of the lamps. Another switch 80, such as a sliding switch, allows control of the device optical power output. The light from lamp 20 is concentrated and directed towards the skin 10 by a reflector 60.

Alternative, the device may couple with a mechanical energy component as shown in FIG. 1c. An exemplary treatment device may comprise an enclosure 1000. Within the enclosure an energy source, 1020 may reside. Said energy source may provide an output of energy. For example said energy source may provide an output of Electromagnetic (EM) energy, Light energy, RF Energy, ultrasound energy, Microwave energy, electrical energy, electrical current, magnetic energy, high or low voltage, vibrational energy, mechanical energy, x-ray energy, gamma ray energy, proton energy, nuclear energy, radioactive energy, ionizing energy, chemical energy.

The enclosure 1000 may also comprise a mechanical of vibrational energy source, 1090. The mechanical energy source may comprise a vibrating motor or shaft, or a source of compressed liquid or fluid such (for example hydraulic or pneumatic source) or a source of compressed fluid, compressed gas or compressed air, all capable when discharged or released, to provide the treatment head (TH) 1040 or treatment Mass 1040 with mechanical energy, for example Kinetic Energy (KE) and mechanical momentum (Momentum=Mass*Velocity). The mechanical energy source, 1010, provides mechanical energy through a delivery chain, 1020 and 1060, to a MEMBER 1070, for example a mechanical coupling head, a treatment head, a hammer, or similar mass, modified and adapted to delivery mechanical impact into the tissue.

Optionally or additionally, the enclosure 1000 may comprise a vacuum source, for example, a vacuum pump or suction pump, 1080, said vacuum source may be connected through a vacuum line 1070 to an optional vacuum ring 1060, said vacuum ring may provide without limitation, a suction, negative pressure, or vacuum to the targeted tissue 1050.

As shown in FIG. 1d, the device 1005 may also comprise a controller, 1010, for example a cpu, or a computer, said controller has a human interface to allow input of information and direction (for example, off and on, charging time, control parameters, limitation parameters, boundaries parameters). Said controller also directs the energy source, 1020 when to emit energy, the mechanical energy source 1090 when to emit mechanical energy, the vacuum source 1080 when to activate and shut off. The Controller 1010 may also connect to the device sensors 1040. The sensors 1040 may comprise imaging members, for example, an Optical coherent tomography (OCT), an ultrasound imager, a light imaging system, a fluorescence imaging system, a microscope imaging, a confocal microscope imager, an atomic force microscope, a temperature sensor, and IR camera or IR sensors, a light sensor, a thermopile sensor, a second harmonic generation sensor, a luminescence light sensors, a pressure sensor, a transducer sensor, a radiation detector, or other sensors known in the art.

The device 1005 may further comprise a vacuum line for transfer of vacuum or transfer of suction or transfer of negative pressure from the vacuum source 1080 to the optional vacuum ring or suction ring 1060, as shown in FIG. 1d. The device 1005 may further comprise a mechanical energy transfer line 1025 for transferring mechanical motion or fluid discharge from the mechanical energy source 1090 to the treatment head and treatment mass 1040. The device 1005 may further comprise an energy transferring member 1027 to transfer the energy from the energy source 1020 to the treatment head 1040 and targeted tissue 1050. The mechanical motion of the treatment head 1070, can, for example, be propelled, by a compressed air, compressed gas, steam, a shock tube discharge, discharge of compressed fluid, discharge of compressed liquid, or even bombardment and discharge of solids, or other similar means. If a compressed air or compressed gas, for example, are discharged from a compressed air or compressed gas container 1025 can be used as source for such compressed substances, and the energy delivery conduit 1020 can be used to deliver said gas or compressed air in order to push the piston 1060 and treatment head, 1070 into the treatment tissue. The piston 1060 can be connected to a spring 1050 that restores the treatment head 1070 and piston 1060 to its un-stretched position, ready for another impact cycle.

The spent, expanded air or gas in the chamber 1065 can then be ejected through a valve 1067, which allows only back flowing gas to exit but not the expanding compressed air or compressed gas in the driving-part of the cycle.

The device 1005 may also comprise energy emitters 1095 or light emitters, 1095 to emit the delivered treatment energy and direct it towards the targeted tissue. Alternatively or additionally, in the device 1005, at least some of the emitter or all of the emitters, 1095 may be replaced by energy ports or light ports 1095 where the energy from the energy source 1020 or light source 1020 can be exit the treatment head and be directed towards the targeted tissue 1050.

Further, the device 1005 may comprise a treatment head (TH) 1040 or treatment mass 1040 that may be moved mechanical force or mechanical energy provided by the mechanical energy source 1090, said TH or treatment mass 1040 is moved repeatedly up and down, or away and into the targeted tissue 1050 so as to apply mechanical force to and impart mechanical energy into the targeted tissue, for example a leg muscle 1050. Said mechanical motion can be periodical with different frequency. For example: about 0.001 Hz to about 1 GHz, about 0.01 Hz to about 100 MHz, about 0.1 Hz to 1 about 0 MHz, About 0.1 Hz to about 1 MHz, about A single shot, About 0.1 Hz to about 100 KHz, about One Hz to about 50 KHz, about One Hz to about 10 KHz, about One Hz to about 5 KHz, about One Hz to about 3 KHz, about One Hz to about 1 KHz, about 3 Hz to about 3 KHz. Optionally or additionally the mechanical vibration of the TH or treatment mass 1050 may be synchronized with the emission of the energy source 1020. Optionally or additionally, the device 1005 may comprise a sensor or imaging systems 1030.

FIG. 1e shows further embodiments of the treatment head (TH). A mechanical energy transferor (MET) 2010 deliver mechanical vibration energy to the mass of the TH 2050, said mass of the TH then accumulate mechanical energy KE=½ M V2, and delivers it to the targeted tissue 2035.

The TH 2050 is also connected to the energy source with an energy or light transmitter member 2020. The energy delivered through the transmitter 2020 is further transferred to the targeted tissue or targeted material 2035, through ducts, or conduits or channels or wires or other means in or around the TH, so it is delivered to the skin, or targeted tissue or targeted material 2035.

For example, channels and ports can be drilled into a TH made of, for example, metal or non-metal substance, said channels and ports may allow, for example, fiber optics or other propagation of the treatment energy, for example, EM energy, light energy, laser energy, electric energy, magnetic energy, fluid mechanical energy, ultrasound energy, microwave or RF energy, or other forms of energy to be delivered to the targeted tissue 2035.

Additionally or optionally, a vacuum or suction may be applied to a vacuum ring 2055, so that a suction or a lift is applied to the targeted tissue or a targeted material or a targeted skin.

Additionally or optionally, a sensor 2030 may be mounted on or around the TH so that properties of the targeted tissue or of the interaction between the targeted tissue and treatment energy and mechanical energy may be monitored. For example, pressure transducers may measure pressure applied to the targeted material. A stress or strain sensor may measure stress and/or strain as a function of the impact of the mechanical and/or treatment energy delivered to the targeted material.

For example, temperature sensor such as Thermocouple, or IR camera, or IR diode, or IR sensor, or other means to measure temperature may also serve to monitor said targeted material temperature.

Sensors or imagers such as OCT, microscope, fluorescence scope, magnifiers, ultrasound sensors, atomic force microscope, luminesce feedback monitoring, or other sensors may also serve to monitor targeted tissue conditions.

Additionally or optionally, resistivity sensors, humidity sensor, scattering and or absorption sensors may also be used.

Finally, a cooling member 2045 may be provided to the TH as well, said cooling member may cover various fraction of the treatment head contact surface with the targeted tissue, to remove energy and cool the targeted tissue. Such cooling member may comprise a thermoelectric cooler (TEC) a cryogen or fluid expansion cooler, a circulating coolant or fluid capable of cooling, an air or gas flow cooling, or other methods of cooling or types of cooling members.

For example a treatment head operating at from about 10 Hz to about 5 KHz, of vibration Pulse repetition rate, or from about 100 Hz to about 4 KHz, or from about 1 KHz to about 4

KHz, or from about 2 KHz to about 4 KHz may be desired.

For example treatment head weighing from about 0.5 lb. to about 10 lb., or from about 1 lb. to about 7 lb., or from about 2 lb. to about 6 lb., or from about 3 lb. to about 5 lb.

The treatment head contact diameter may be for example, form about 0.1 inch to about 10 inch, or from about 0.2 inch to about 7 inch, from about 0.5 inch to about 5 inch, or from about 0.5 inch, to about 3 inch, or from about 0.5 inch to about 1 inch.

The treatment head contact area may be from about 1 mm2 to about 100 cm2 or from about 5 mm2 to about 50 cm2 or from about 10 mm2 to about 30 cm2, or from about 50 mm2 to about 20 cm2, or form about 1 cm2 to about 10 cm2.

FIG. 2b illustrates a device 200 for hair reduction which allows performance of multiple tasks together. An element 205 capable of removing hair shafts from the surface of the skin by conventional means (such as a razor, wax dispenser/remover, a tape dispenser or other means for removing hair by conventional means) leads the treatment head as it makes contact with the target skin area 10. The hair removal element 205 is followed by a cleaning element 220 capable of removing any excess hair, debris, dirt, dead skin or any other undesired substance that may be covering the skin surface.

The area targeted for treatment is then exposed to a flash lamp 20. The flash lamp 20 can be, for example, of the same type used in disposable cameras and digital camera flash. The flash lamp is powered by one or more batteries 30 and one or more capacitors 40, and its operation is controlled by a small circuit board 50. The batteries 30, capacitors 40, and circuit board 50 can be, for example, similar to those used in a digital camera or in disposable cameras with flash such as the Kodak disposable extended range camera.

Alternatively, the capacitors 40 can also be powered by direct connection to 110V or 220V power line from a domestic wall outlet of a domestic or industrial line. A switch 70 allows charging and firing of the lamps. Another switch 80, such as a sliding switch, allows control of the device optical power output. The light from lamp 20 is concentrated and directed towards the skin 10 by a reflector 60.

Another cleaner 260 then follows the action of the light pulse and acts to remove any additional debris or dispense nutrients, substances capable of rejuvenating the skin, after shave, lotions, potions colognes or any other substance one may wish to deliver to the skin and enhance the hair removal treatments.

Alternatively, as also shown in FIG. 2b, the device may also include a dispenser 230. The dispenser 230 is positioned immediately after the cleaner 220. The dispenser 230 is capable of dispensing and driving into the skin a substance with absorption in at least a portion of the spectrum of the light emitted by the lamp 20. The substance capable of light absorbing (SCLA) is also massaged or driven into the skin pores (the hair follicle pores) by the action of the dispenser 230. Following the action of dispenser 230 and as the treatment head of device 200 is moved in the direction of the treatment 274, yet another cleaner 240, optionally, is installed to remove the excess of the SCLA form the surface of the skin allowing most of the SCLA to be removed and substantially mostly only the pores and hair follicles openings retain some SCLA. The device 200 is moved substantially until a distance substantially equivalent to the size of the lamp window and then stops. The lamp 20 is then fired by pushing the trigger button 70 after adjusting the power level to the desired level with the power adjustment switch 80. The device is then pushed forward to the new location. As the device 200 is pushed forward where the treatment head is aligned with the new desired treatment location, yet another cleaner 260 provides the surface with a final cleansing and possibly drives in nutrients, healing substances, lotions, medicine or healing drugs, colognes and scented substances, substances capable of skin rejuvenating, aftershaves, or any other substances that may be desired.

The device reduces the presence of hair on the body, the device as shown in FIG. 2b comprises: a hair remover 205 to remove the surface hair form the area of the skin target for treatment. Such a hair removal may be a razor, for example, a single blade, a five blade or four blade razor such as the one used by Gillette or Schick, razors. Alternatively, the hair remover 205 may be composed of an adhesive tape, or contact bar with adhesive properties capable of attaching itself to hair and removing the hair when moved away with the hair attached to it. The device further comprises a cleaning element 220 to clean the target area on the surface of the skin, the cleaning element can be made, and for example, form a wet rubber, or a brush, capable of removing cut, or plucked hair. The device may be further constructed with a dispenser 230. Dispenser 230 is capable of dispensing a substance capable of absorbing at least some of the energy of the hand held light source. The dispenser may be attached to a reservoir 265 of the absorbing substance; the reservoir 265 is located within the apparatus enclosure 200. The absorbing substance can be any suitable conventional material which absorbs the energy from the light source.

A massager or substance-driver 240 capable of massaging the substance on the target area of the skin or driving at least some of the substance into the skin. The hair remover or hair plucker 205, the Cleaner 220, the dispenser 230, and the substance driver 240 may all be mounted ahead of a light or energy source capable of delivering light into the pretreated skin. Thus, if the direction of motion of the device along the skin 10 is shown by the arrow 274, these elements are mounted as shown in FIG. 2b, so that their action on a given portion of the skin 10 precedes the application of the light source 20 above that portion of the skin and activation of the light source 20 over that portion of the skin 10.

In a further embodiment of a cleaner, such as the cleaner 260 depicted in FIG. 2b, comprises a conditioning element 260 capable of applying conditioning creams, lotions, or any other substance to enhance the skin appearance and condition. For example, the conditioning element may be a nozzle connected to a reservoir containing lotions, nutrients, vitamins, hydrating to the direction of motion of the device, shown by the arrow 274, and preferably trailing the light source 20 so that the irradiation and pre-treatment occur prior to dispensing of the conditioning substance from the conditioning element of the cleaner 260 as shown in FIG. 2b.

In a further embodiment, a method for reducing the presence of hair on the body is provided, the method comprises removing the hair from the target area on the surface of the skin targeted for treatment, cleaning the target area on the surface of the skin, applying to the target area of the skin a substance capable of absorbing at least some of the energy from a handheld light source, massaging the substance on the target area of the skin, cleaning the target area on the surface of the skin, and activating the light source from the handheld light source to shin on the target skin area. The method may further comprise applying on the skin a substance capable of enhancing the skin condition and enhancing the skin appearance.

Another embodiment can include a flash lamp light source; the flash lamp can be similar to the Perkin Elmer 2033 xenon discharge lamp that discharges up to 28 joule of electrical energy, or other flash xenon lamps that discharge up to 10 J of electrical energy, or a krypton discharge lamp. Such lamps can give off several joules of optical energy so that the optical energy density on the surface of the skin can range from 0.01 J/cm2 to as much as 400 J/cm2 and preferably from about 0.1 J/cm2 to about 20 J/cm2. Such lamps are typically pulse and the pulse duration is from about 0.01 ms to about 500 ms and preferably 0.1 ms to about 10 ms. Emission spectra is broad band and can range from about 300 nm to about 1200 nm and preferably from 400 nm to about 1100 nm. The device energy discharge repetition rate is form about 0.01 hertz to about 50 Hertz and preferably form about 0.1 pulses per second to about 10 pulses per seconds. Practically the device could be fired a few times per seconds to achieve the desired temperature elevation in the hair structure to cause disruption of hair growth, yet to prevent collateral damage to the skin or other structures of the hair follicle.

In a further embodiment, the device of FIG. 2b can also be used where at least some of the absorbing substance is allowed to remain on the surface of the skin and is not removed from the skin. This will create a thermal effect on the surface of the skin and principally at the opening of the pores. The heating of the top of the follicle will allow some thermal energy to diffuse and traumatize lower portion of the follicles.

Alternatively or additionally, as shown in FIG. 2c, the mechanical energy delivery is accomplished through a motor 1010 (mechanical motor, electrical motor, steam motor, gas motor, stepper motor, or other types of motors known in the art) which generate lateral motion in the shaft or mechanical motion translator 1030, for example by rotating a series of extended protrusions 1033, or “wings” 1033, said “wings” gradually push the piston 1030 further to the left as the motor 1010 rotates. Until they pass their maximum displacement and, for example, a spring load return the piston 1030 to its original position. Alternatively, other types of gears, chain of gears wheels, rack and pinion assemblies or other means known in the art to translate rotational motion into a piston 1030 or other mechanical motion translators 1030, can be used.

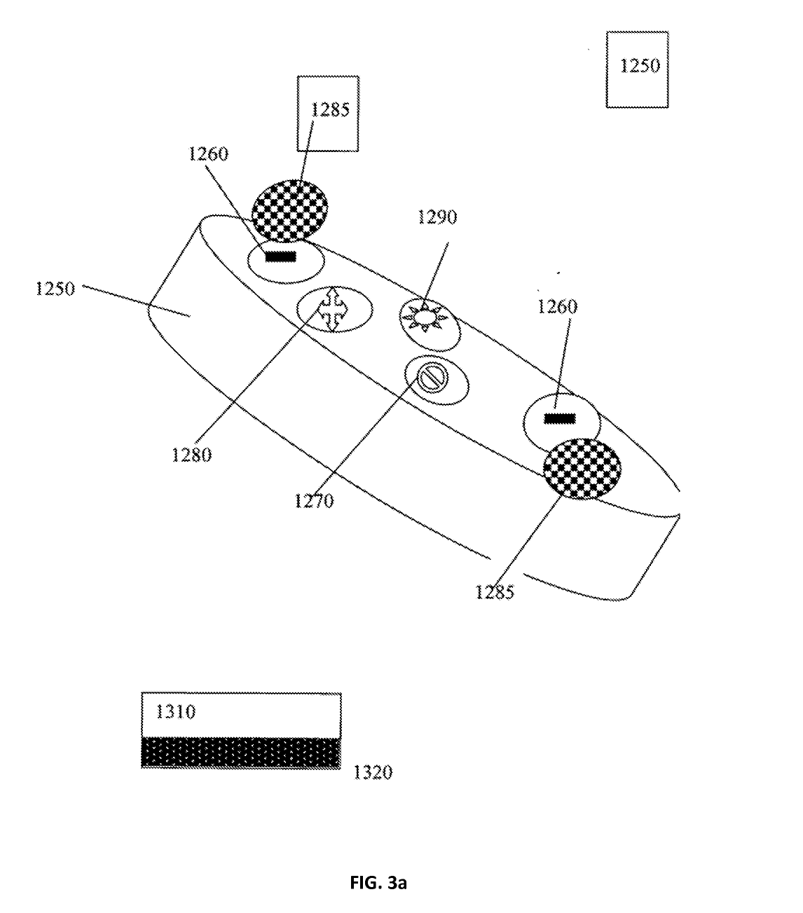

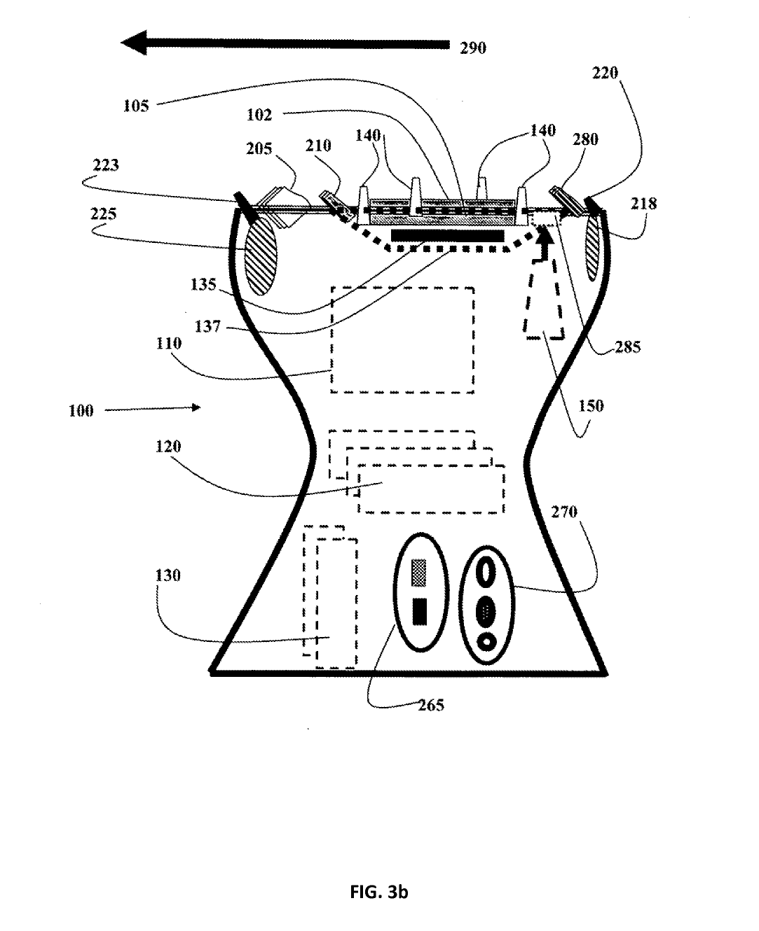

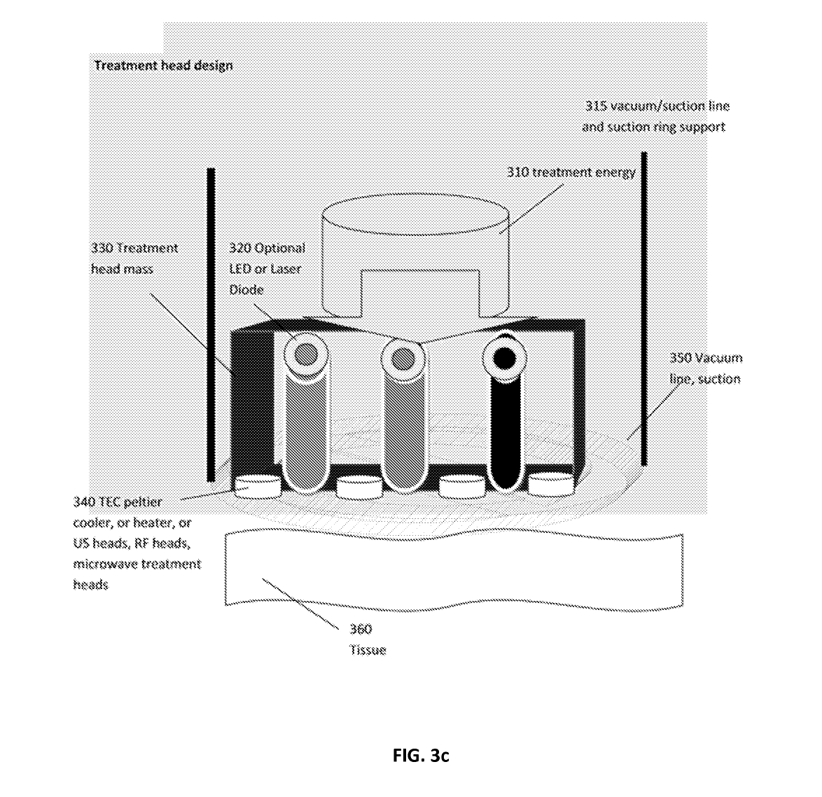

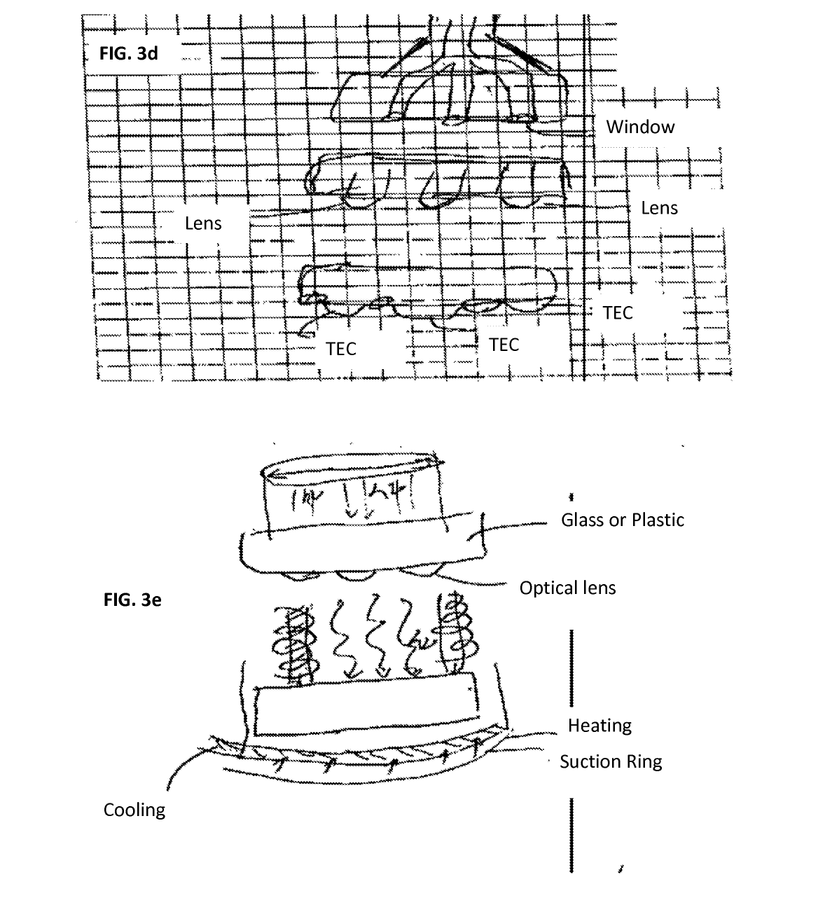

FIG. 3a shows a treatment head 1250 which contains a combination of the following elements: a plurality of flash lamps 1260, a plurality of electrical heating elements 1270, a plurality of mechanical scraping or buffing elements 1280, a plurality of suction devices 1290. The treatment head 1250 may contain some of the above elements. For example, it may contain only a plurality of flash lamps 1260. Or it may contain a plurality of electric treatment windows, or it may contain a combination of both plurality of flash lamps and electric heating treatment windows. FIG. 3a also shows a plurality of partially or fully absorbing layers 1285 over the light generating elements 1260 or flash lamps 1260 and between the light generating elements and the targeted skin area. In an embodiment, the absorbing layer can be placed in front of the lamp or removed from its intermediate position between the lamp window and the surface of the skin. Furthermore, the absorption layer can be made of the following components (see FIG. 3a).

A backup layer 1310 that provides some rigidity, and a front layer of absorbing material 1320. The backup layer can be a transparent layer and can be made, for example, from glass or high temperature plastic, capable of sustaining the temperature generated by the device at the absorbing layer and without deforming or substantially deteriorated.

Alternatively, the absorbing material can be embedded or deposited or painted on the surface of the backup transparent layer 1310 on the surface placed against the skin. In an alternative embodiment, the absorbing material can be made of carbon particle coated over a substrate layer 1310. Or the absorbing particles, for example carbon particles, can be embedded in a transparent layer, for example a layer of glass or plastic. Alternatively the glass or plastic can be etched or scratched with grooves that retain the absorbing material at its surface. The absorbing material should be deposited close to or on the surface of the substrate layer 1310 that is closer to the target skin surface. Another embodiment utilizes a thin heat conducting layer, for example a layer of metal such as gold or copper or other heat conducting materials, as the substrate 1310, with an absorbing layer 1320 placed on the side which is farther away from the target skin surface and closer to the light energy source. In this embodiment, the absorbing layer 1320 can be etched, painted, embedded or coated onto the metal substrate layer 1310. Alternatively the substrate layer 1310 can be machined or conditioned (e.g. with electron beam or laser beam, excimer laser beam, chemical etch, or any other method allowing the surface of a metal to trap light energy or enhanced the surface of the metal to absorb the light energy). The light energy would be rapidly conducted towards the skin surface in contact with the metal layer 1310 on the opposite surface of the high absorbing layers.

FIG. 3b shows the components for a possible flash lamp device 100. The device 100 includes a flash lamp lens 105 such as the Perkin Elmer BOAC 3022, COD 0013, COA 2018, or similar products used in digital cameras such as the Pentax, or the single use camera made by Kodak or Fuji or similar products. A window 102 is used to cut out harmful UV radiation and protect the user from electrical contact. The window 102 is therefore made of a transparent, non-uv transmitting, and electrically insulating material. A PCB board 110 contains the electronic components that control the operation and triggering of the flash lamps, as well as the pulse forming electronics. Safeguard projections in the form of protruding guards 140 are provided, for example, on the window 102 or on the treatment head and around the window 102. The protruding guards 140 are part of a control element or controller to prevent inappropriate activation of the device, and are designed to be physically compressed by contacting with the skin. The protruding guards 140 can be made from a biocompatible material, for example aluminum, or hard plastic material, or rubber. They can be spring loaded aluminum rods with enlarged footings—the end that makes contact with the skin. The protruding guards 140 act as sensing elements to determine the distance of the flash lamps from the skin. When the protruding guards 140 are depressed, they make close an electronic circuit that allows the energy source to be activated. If the protruding guards 140 are not depressed, the device cannot be activated.

The requirement for physical compression by the force applied by the skin to the protruding guards ensures that application of the device 100 against the eye or sensitive or damaged skin is painful and difficult and, therefore, would be less likely to occur. A plurality of batteries 130 provides the energy source for the operation of the lamp. Alternatively, an AC adaptor or an electrical wall plug may be used to power the device. The energy from the batteries 130 is then used to charge a plurality of capacitors 120 which store the energy within them. The discharge of the capacitors 120 energy is willfully triggered by a triggering switch 270 which is connected to the PCB control board 110 to allow a discharge of the energy through the flash lamps behind window 102. The energy then travels through the skin to achieve selective PHOTOTHERMAL or other mechanism of action on the skin hair components to allow disruption or reduction of hair growth rate and hair growth characteristics. The triggering switch 270 may contain three buttons for low, medium, and high power, respectively. The on/off switch 265 allows turning on or shutting down the device. A lamp 135 and a reflector 137 allow the light shining towards the device to be redirected towards the skin. A laser source 150 may allow a combination of dual wavelength or concentrated second wavelength to act on the hair follicle to maximize the effect of the light sources on the skin. The laser beam may be further modified with an optical element 285. The optical element 285 may be a defocusing lens to create a broader beam, an optical diffuser capable of scattering the light so that the beam is no longer focused, or a focusing lens made of glass or plastic, the focusing lamp allows enhanced power density concentration on the targeted skin and hair.

If the direction of motion is indicated by the arrow 290, the following components in FIG. 3b will constitute one embodiment. For example, a reservoir 225 may dispense a cleaning and/or lubricating solution through a nozzle 223 made of metal (such as aluminum) or plastic. The nozzle 223 is positioned between about 0.5 to about 4.5 mm above the skin surface during operation of the device. A plurality of blades 205 may shave the surface of the skin. The blade(s) 205 may be made from an aluminum or stainless steel and be sufficiently sharp to remove hair form the surface of the skin. Alternatively, the blade 205 may be made from stainless steel and may be placed slightly higher than the surface of the skin so that it leaves a hair shaft of height of about 0.1 mm and about 4.5 mm above the skin, and preferably about 0.2 to about 0.5 mm of hair shaft above the skin. Such remaining hair shaft may be useful in removing the treated hair once it has been sufficiently damaged and weekend and can be easily removed from the surface of the skin. The plurality of blades 205 can be stacked as in the gillete five-blade system. The blade(s) 205 can also be vibrating to induce a better shave and also to induce better product or absorbing substance delivery.

A further component of the embodiment shown in FIG. 3b is a first cleaner blade 210 that precedes the lamp 105 with respect to the direction of motion, and a second cleaner blades 210, 280 that can trail the lamp 105 with respect to the direction of motion 290. The cleaner blade can remove some of the absorbing material deposited prior to light interaction or some of the conditioning material deposited prior to treatment. In addition, a skin conditioning or aftershave reservoir 218 and delivery nozzle 220 can be incorporated to complete the treatment with a delivery of aftershave or hair and skin conditioning substances.

FIGS. 3c-3e illustrate a general sketch of the device treatment head and some of its possible components. For example, a treatment head can comprise a mass 310 capable of imparting mechanical impact to the skin (for example a force of . . . , or a mechanical impact capable of deforming the tissue (at the point displaced most) by at least 2%, 4%, 10%, 15%, 20%, 30% . . . . ))

In another example, a treatment head can comprise a mass 310 capable of imparting mechanical impact to the skin (for example a force of . . . , or a mechanical impact capable of deforming the tissue (at the point displaced most) by at least 0.01 mm, 0.1 mm 0.5 mm 1 mm 5 mm 10 mm 20 mm 25 mm 30 mm 40 mm 50 mm 60 mm 70 mm . . . 100 mm, 150 mm 200 mm 250 mm 300 mm, optionally or additionally, the treatment head also has a suction cap, optionally or additionally, the treatment head also has a TEC, optionally or additionally, the treatment head also has a heater, optionally or additionally, the treatment head also has a EM energy source, optionally or additionally, the treatment head also has a light source, optionally or additionally, the treatment head also has a flash lamp source, optionally or additionally, the treatment head also has an LED light source, optionally or additionally, the treatment head also has a laser source, optionally or additionally, the treatment head also has a laser diode source, optionally or additionally, the treatment head also has a microlenses, optionally or additionally, the treatment head also has an incoming EM beam and microlenses, optionally or additionally, the treatment head additionally or optionally comprises an electrophoresis source, optionally or additionally, the treatment head also has an ultrasound source, optionally or additionally, the treatment head also has an imager, optionally or additionally, the treatment head also has an OCT, optionally or additionally, the treatment head also has an ultrasound imager, optionally or additionally, the treatment head also has an ultrasound energy source, optionally or additionally, the treatment head also has an RF energy source, optionally or additionally, the treatment head also has a microscope imager, optionally or additionally, the treatment head also has a fiber optic energy source, optionally or additionally, the treatment head also has a hollow wave guide as an energy source, optionally or additionally, the treatment head also has a LIBS material analyzer, optionally or additionally, the treatment head also has a fluorescence imaging, optionally or additionally, the treatment head also has an ultrasound energy source, optionally or additionally, the treatment head also has an RF energy source, optionally or additionally, the treatment head also has a microscope imager, optionally or additionally, the treatment head also has a fiber optic energy source, optionally or additionally, the treatment head also has a hollow wave guide as an energy source, optionally or additionally, the treatment head also has a LIBS material analyzer, optionally or additionally, the treatment head also has a fluorescence imaging.

As explained above, the need exist for method, devices, systems and/or apparatus/apparatus to treat tissue with both Energy source, (for example penetrating energy sources and/or surface deposited energy source) in combination with mechanical and/or electrical tissue modifying systems and/or method.

FIGS. 3c-3e shows an optional treatment head design. The Treatment head can comprise, for example, a treatment energy source, for example a laser light source, an LED light source, a flash lamp light source or RF energy source or other EM energy source. The beam from the treatment energy source is coupled through a treatment head mass 330 which can be transparent or may be opaque or partially transparent to said treatment energy the treatment energy source passes through the treatment head mass either because the treatment head mass is transparent or through specialized conduit or channels provided or drilled in the treatment head mass.

Additionally or optionally there are provided 340 TEC Peltier cooler, or heater, or US heads, RF heads, microwave treatment heads. Additionally or optionally a suction or vacuum provided by the ring 353 can be attached to the skin 360 on one side and to the main body of the device through a vacuum/suction line that also provide the suction or vacuum to the vacuum source.

The treatment mass passes through the suction ring and can impact the skin at a rate of between a single pulse and 1 GHz, or more preferably between 1 Hz and 100 MHz, or more preferably between 2 Hz and 1 MHz, or more preferably yet, between about 2 Hz and about 10 KHz. Additionally, an exemplary mass impact rep rate may, for example, be between about 1 Hz and about 50 Hz.

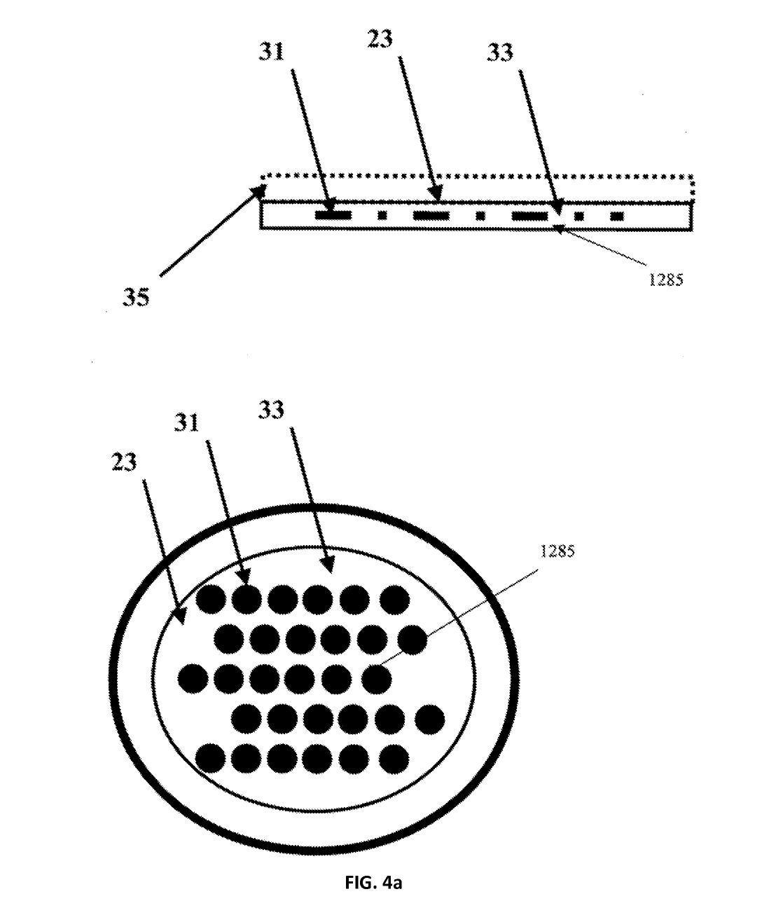

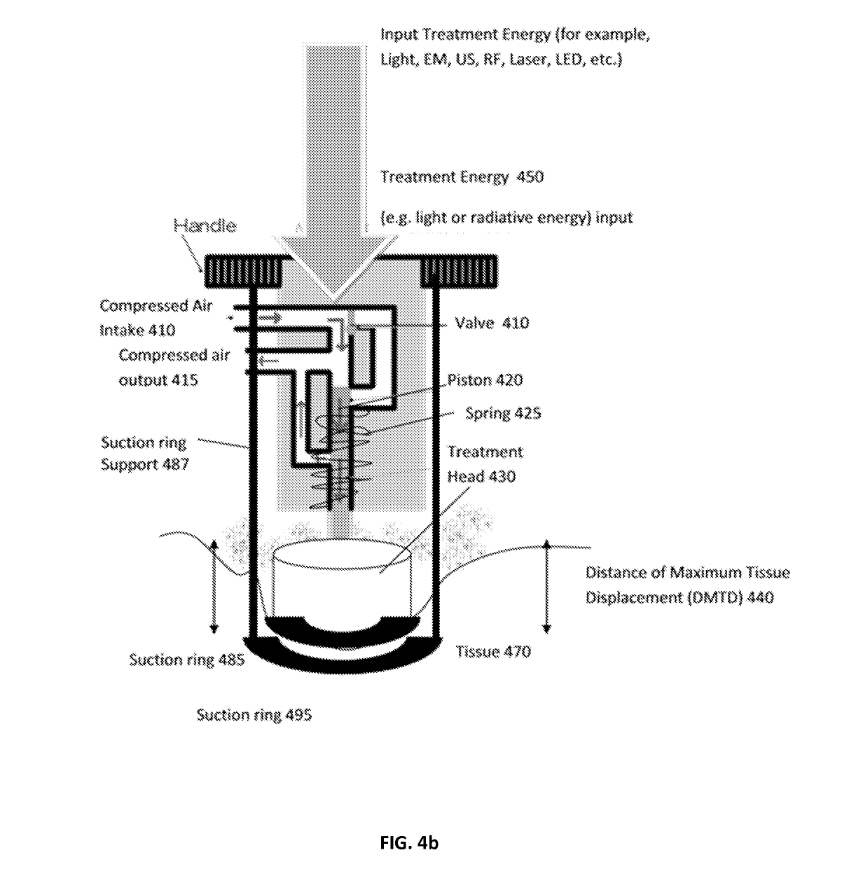

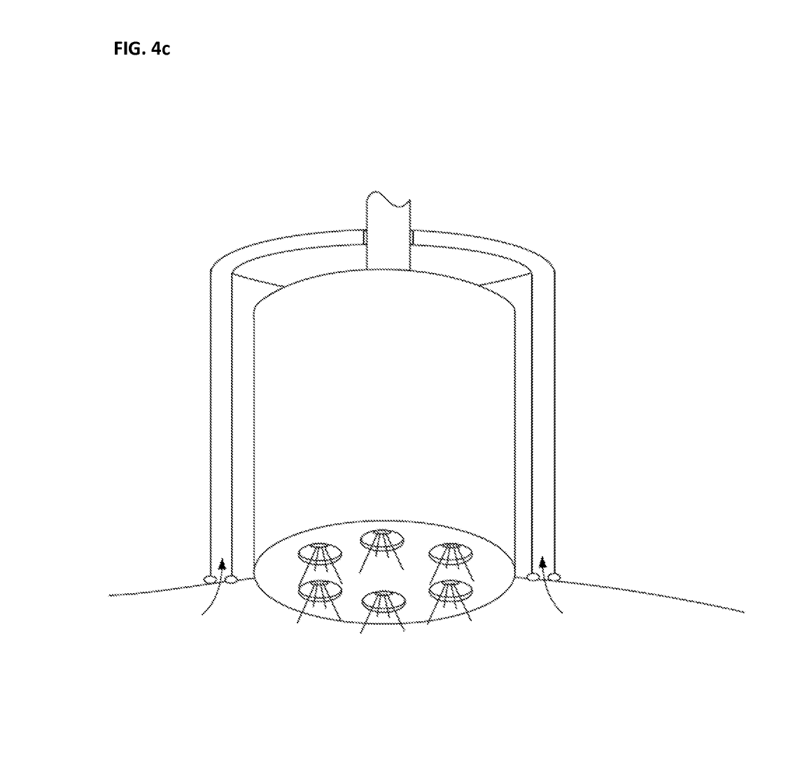

FIG. 4a shows a front and a side view of another embodiment of the absorbing layer. The absorbing layer can be made rigid, electrically insulating with absorbing capabilities ranging from about 0% (full transmittance) to as much as about 100% absorption (full absorption). For example a transparent layer 35 can be attached to the absorbing layer between the absorbing layer and the light source or lamp. Said absorbing layer can comprise for example a high temperature glass or plastic material doped with absorbing material. Alternatively it can comprise a metal layer capable of absorbing the flash lamps light and also coated with an optically transparent and electrically insulating layer between said metal layer and the flash lamp assembly.

FIGS. 4b-4c illustrate a mechanism of delivering mechanical energy through the use of compressed air or compressed gas (generally referred as a compressed fluid in the present description of FIG. 4b). An intake port 410 allows the compressed fluid to come in and drive the piston or pile driver 420 down so it propels the treatment head mass (THM) 430 towards the tissue. After imparting its kinetic energy to the tissue, a restorative force 425, for example a spring 425, pulls the Treatment Head 430 back to its original position. As the treatment head (TH) 430 is pulled back up, the piston 420 expels a the spend (or expanded/spent) fluid, for example air or gas, out through the output port, 415 and is ready for another delivery cycle.

As is also shown in FIG. 4b, the treatment head 450 compresses the treated tissue 470 and deliver other forms of energy (other than mechanical energy) for example, EM energy, light energy, laser energy, RF energy, ultrasound energy, thermal energy, electric energy, Magnetic energy, or other forms of energy. The TH can also deliver drugs, medicine, nutrients vitamin and other beneficial substance. As shown in FIG. 4b the TH displaces the tissue by compressing it.

The distance of maximum tissue displacement (DMTD) 440 is also shown. Additionally or optionally, the treatment head can also provide vacuum or suction to the tissue. Such suction allows better attachment and contact of the TH with the tissue and further impact the skin mechanically. The suction 485 can be provided, for example, by a suction ring 485 attached to the TH outer rim or, for example, separated from the TH and outside the TH rim, 495. A suction ring support 487 may be included to support the suction ring and provide a vacuum line.

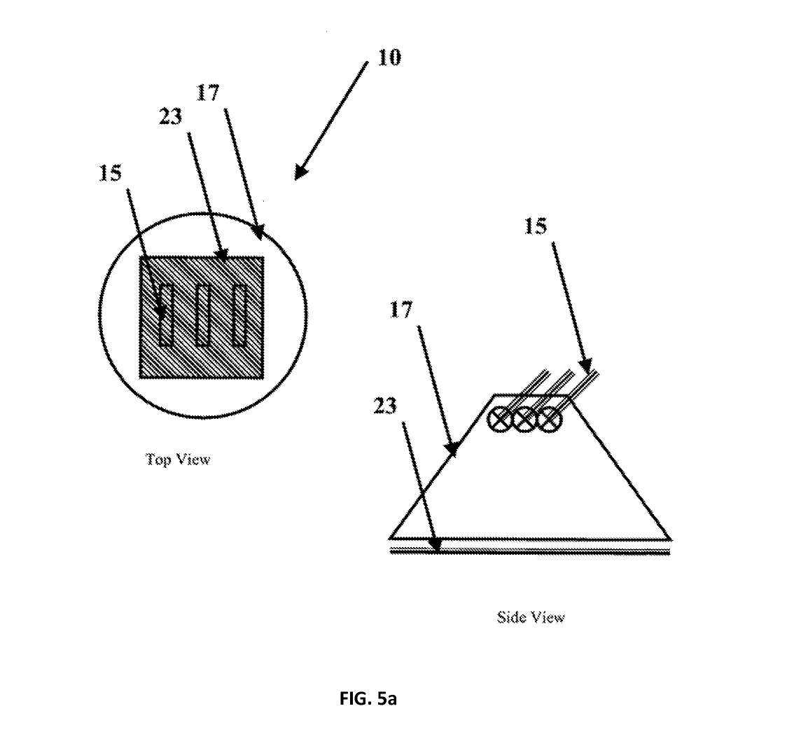

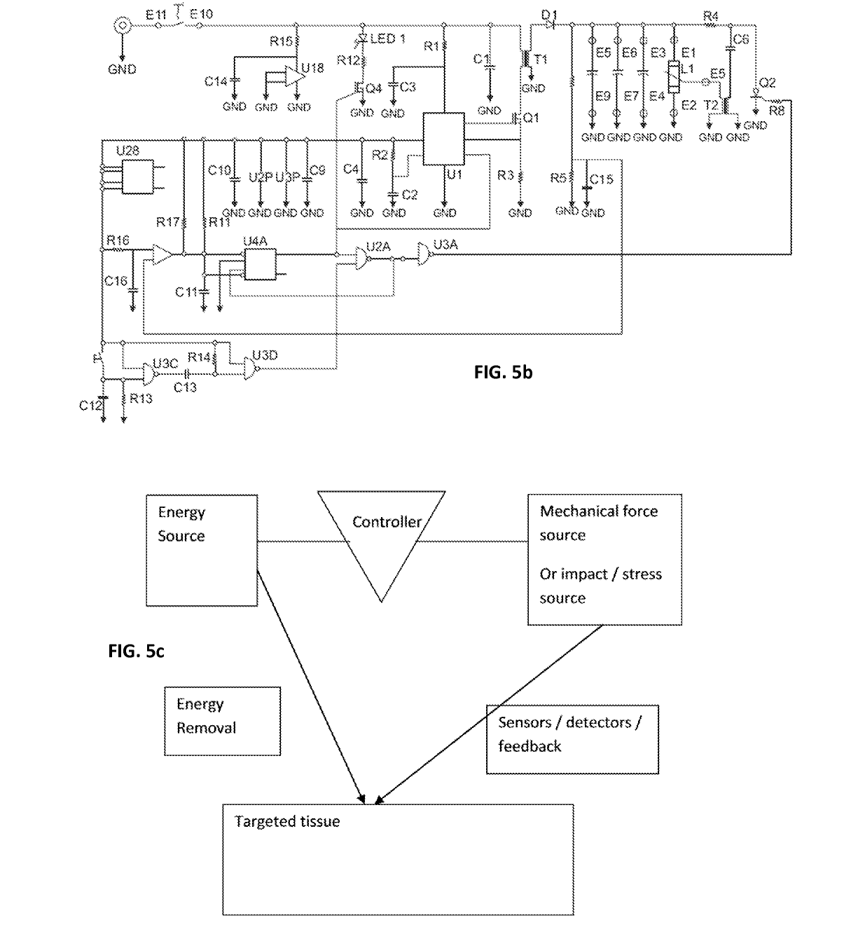

FIG. 5a further shows an embodiment of absorbing layer possible composition, wherein the high absorbing film 23 between the lamps 15 and the skin surface is made of partially transmitting material, for example, part of the film contains high absorbing substance 31 to absorb the light of the lamps, while other portion 33 of the film allows at least some of the optical energy to penetrate through to the skin. This configuration will allow part of the light energy to be converted into heat at the skin surface and directly heat the top layers of the skin, while some of the light is allowed to propagate to deeper skin layer where a gradual absorption by skin cell heats up deeper skin tissue. In addition, some of the light that penetrates deeper skin tissue may be preferentially absorbed by skin components (for example blood vessels, or pigmentation) that may be targeted for destruction or alteration. The device in this embodiment can, therefore, serve for both skin surface treatment as well as targeting of deeper layers skin conditions.

FIG. 5a shows an alternative embodiment of a skin treatment head 10. In this embodiment, a single reflector 17 encloses a plurality of lamps 15 thus allowing increased energy output from each reflector 17 in the treatment head 10. In this example, each reflector has three lamps. Layer 23 is an absorbing layer.

FIG. 5b illustrates yet another exemplary circuit diagram for powering the Perkin Elmer 20 BOAC 3022 lamps. To achieve the specified energy density range, an exemplary electronic circuit shown in FIG. 5b may be used with the following components:

TABLE 1

| U1 | PWM Voltage Regulator | | |
|---|---|---|---|
| U2 | Dual D Flip Flop | | |
| U3 | Quad NAND | | |
| U4 | Dual Comparator | | |
| J1 | Jack, Power | | |
| Q1 | Transistor, MOSFET, N- Channel | | |
| Q2 | SCR | | |
| Q4 | Transistor, MOFSET, N-Channel | | |
| R15 | Resistor, Thick Film, 1110 W, 1% | 100 | ohm |
| R1 | Resistor, Thick Film, 1110 W, 1% | 301 | ohm |
| R12 | Resistor, Thick Film, 11lO W, 1% | 1.00K | ohm |
| R8, 14, 16 | Resistor, Thick Film, 1110 W, 1% | 1O.OK | ohm |
| R5 | Resistor, Thick Film, 1110 W, 1% | 14.3K | ohm |
| R2, 11, 13, 17 | Resistor, Thick Film, 1I1O W, 1% | 100K | ohm |
| R3 | Resistor | 0.05 | ohm |
| R6 | Resistor, Carbon Film, ¼ W, 1% | 1.00M | ohm |
| R4 | Resistor, Carbon Film, 1I4 W, 5% | 4.7M | |
| C2 | Capacitor, Ceramic Chip, NPO | 500 pF, | 50 V |
| C3, 4, 9-15 | Capacitor, Ceramic Chip, Y5V | 0.1 uF, | 16 V |
| C16 | Capacitor, Ceramic Chip, Y5V | 1 uF, | 16 V |
| C6 | Capacitor, Polyester | 0.047 uF, | 400 V |
| C1 | Capacitor, Aluminum Electrolytic, | 470 uF, | 16 V |
| | Radial | | |
| D1 | Diode, Silicon | | |
| T1 | Transformer, Flvback | | |
| T2 | Transformer, Trigger | | |
| LED1 | Diode, Light-Emitting, T-1¾, | | |
| | Green | | |
| S2 | Switch, Pushbutton, SPST | | |

Such an electronic circuit can be used to drive a plurality of flash lamps such as the Perkin Elmer BGAC 3022, CGD 0013, CGA 2018, or similar products used in digital cameras such as the Pentax, or the single use camera made by Kodak or Fuji or similar products with energy densities ranging from about 0.01 J/cm2 to as much as about 50 J/cm2.

As is shown in FIG. 5c, in some embodiments, a device may comprise of various members that cooperate to function. These member include, without limitation, 1) and energy source, 2) a mechanical force and/or mechanical stress, or mechanical strain applicator, 3) a coupling member, to allow positioning and/or attachment of the device to the targeted tissue, 4) a lens, window, mask, kinophorm plate, diffractive member, phase plate, Fresnel lens, filter, attenuators, or scanners to direct or modify the output beam, 5) a cooling or energy removal member, 6) a control and feedback member, to control and synchronize and adjust operation of all members of the device,) a feedback member, to feedback information between the targeted tissue, the effect created in the tissue, and the controller, said controller controls and affect the operation of the device, the device energy source, and the device mechanical force applicator, and/or mechanical stress applicator, and/or mechanical strain applicator; optionally or additionally, the device may also comprise, 7) a cooling and/or heat delivery system, for example, a thermoelectric cooler with switched polarity, a contact sapphire tip cooler, compressed gas, or compressed cryogen that can be discharged and remove or deliver heat to the targeted tissue.

For example an exemplary device of the present disclosure may comprise the following member:

1) An Energy Source May Comprise:

EM Energy source, A Laser, A Radio wave source, A Microwave source, An X-ray source, An Incoherent EM Energy Source, An incoherent broad band light source with multiple color or multiple wavelengths, A flashlamp Light source), A halogen Light source, An LED Light source, A xenon lamp light source, A discharge lamp light source.

2) Mechanical Force Applicator or Stress Applicator

The devices and methods of the present disclosure may include a member capable of providing mechanical force or mechanical impact or mechanical stress, mechanical compression, suction, and similar mechanical effect. Such members may comprise a mass, a hummer head, a protruding member, a heavy mass, or a substance shape to be able to apply the needed pressure on the tissue to modify said tissue volume and other tissue characteristics. The mechanical force providing member may comprise, a vacuum pump and suction ring, a transducer, lenses, protruding guards, protruding pins, a window, a window with modified and/or structured surface, a spring loaded pins and/or spring loaded mechanical protruding guards.

3. A Coupling Member

In some embodiment a coupling member may be used to attach the device to the targeted tissue. Such coupling member may comprise for example, a hollow cylinder where the piston or mechanical impact head, or hummer head can slide in and out, and where the bottom of the cylinder, i.e. the region making contact with the skin, comprises a suction ring, connected to a vacuum source or a pump, so that a suction or negative pressure can be applied to said ring and said suction in the ring at the bottom of said cylinder can create a suction contact, or vacuum contact, or a tight seal with the circumference of the tissue to be treated.

The device may also comprise mechanical contact applicators such as clamps and rollers, or mechanical transducer, actuators, vibrators, oscillating mechanical masses, positioning devices, suction and/or vacuum line to hold and/or compressed the tissue. Targets for the delivered energy may be water, melanin, blood, hemoglobin, dye or carbon based substances, fat, lipids, or externally injected chromospheres.

Additionally or alternatively, some of the embodiments of the present invention may include a mechanical transducer to deliver the mechanical impact of the device, said mechanical transducer may comprise a mass capable of delivering the required impact, the mass may be made of one or more of the following material (without limiting the mass composition to other materials known in the art): Steel, Iron, Copper, Aluminum, Titanium, Granite, Ceramic, Plastic, Brass, Gold, Silver, Glass, Other materials known in the art and capable of delivering an impact to the tissue.

Impact, I, in physics is equal to the change in momentum:

I=d(mV)=Fdt

Where m is the mass of the mechanical transducer, V is its velocity, mV is the momentum (often designated by P) of the moving device, d (mV) is the change in momentum of the mass of the device, F is the force needed to bring about the change in moment, And dt is the time duration that the force is occurring.

The surface of the mass or mechanical transducer that come in contact with the targeted tissue, may be shaped or structured or constructed in ways consisted with the principles of the present invention.

For example, without limitation, the surface of the mass or mechanical transducer may comprise one or more of the following structures and/or shapes: A flat surface, A rough surface, Corrugated surface, A surface with a protruding guards (PG), An array of PG, An array of pins, A U-shaped transducer, A clamp-shape transducer, A clamp shape transducer with pins or PG protruding out of the clamp, A transducer moved or powered by a mechanical force, hydraulic, air hummer, pneumatic, pistons, hummer-head, springs, electrical motor, mechanical motors, other motors, screws, clamps.

The PGs or pins may be as short as a few tens of micrometers or up to about a few cm. Alternatively or additionally, PGs or pins may be as long as a length sufficient to prevent a direct contact of the their bases or the area around the PGs or pins bases, with the tissue. In another embodiment, the PGs or pins may be of varying lengths. Alternatively or additionally if the PGs or tips surface area at the contact point with the tissue is Ap, then the total tissue surface area covered by the PGs or pins is Aptot=N*Ap, where N is the total number of pins.

Thus, if A is the total area covered by the treatment head, the percent area covered by the PGs or pins is: Fractional area covered=Fa=Aptot/A

And Fa can be from about 0, i.e. no PGs nor pins, i.e. a situation corresponding to a flat surface of a contact mass, or a contact hummer or piston or a hummer head, To about 95%, a situation corresponding to a large number of pins which almost create a continuum of adjacent PGs or pins.

In another embodiment, the fractional area covered Fa range from about 0 to about 50%. The ranges of the contact surface Ap, was described elsewhere in the specification. In another embodiment the fractional area covered, Fa, may range from about 10% to about 50% or from about 5% to about 80%, or from about 20% to about 60%.

Suction Ring.

A suction ring or a contact ring, or other mechanical vacuum source that encircle the contact area of the device, may be employed. For example, if the contact mass or hummer has a cylindrical shape of radius R, a suction ring with a radius R+dR, may be used to at least create a contact with the tissue, or to create both a contact and a suction attachment with the tissue.

The ring then completely enclosed the contact surface of the piston or contact mass or hummer head and may be used to create an airtight vacuum seal with the targeted tissue. The ring contact surface with the tissue may be from about 0.5 mm to about 3 cm and more preferably from about 0.3 mm to about 2 mm. The ring contact surface may be displaced further outside and away from the center of the contact surface or piston or contact mass. For example the ring may be displaced further out from the outer edge or outer lip of the contact mass, by from about 0.1 mm to about 5 cm, and more preferably, from about 0.5 mm to about 1 cm, or from about 1 mm to about 3 mm.

The length of the ring (or dimension of the ring from surface to where said ring contact the base of the device may be about the same as the length of the PGs or, alternatively, may be the length of the fully depressed PGs where said PGs are fully depressed by the tissue.

The source energy may be coupled to the tissue through a window. The window may be made of transparent material (for example, glass or plastic, sapphire, diamond, or other transparent materials). It may also be made of a filter, or a combination of transparent material, variable transmission material and opaque material. The window thickness may, in some embodiments range in thickness, from about 0.1 mm to about 5 cm, or from about 0.5 mm to about 2.5 cm, or from about 1 mm to about 10 mm, or from about 1 mm to about 5 mm.

In some embodiments, a vacuum source may be incorporated to create mechanical suction or mechanical attachment with the tissue. The suction may comprise of an suction or an aperture that can be connected to a tube which lead to a low pressure generator such as a pump. Multiple apertures may be utilized and the creation of low pressure or vacuum attachment may be generated by several techniques or methods known in the art.

4) A Lens, Window, Mask, Kinophorm Plate, Phase Plate, Fresnel Lens, Filter, Attenuators, or Scanners to Direct or Modify the Output Beam.

5) Cooling.

In some embodiment cooling may be incorporated into the disclosed invention. Cooling may provide comfort and may prevent thermal damage and burns. The cooling may be applied to the targeted tissue prior during or after the application of the source energy, and may be applied to the tissue prior, during, or after the application of the mechanical compression of the tissue. In some embodiments the cooling may comprise application of Thermoelectric cooler (TEC), and in some embodiment cooling may comprise a convective cooling fluid such as cooling gas or liquid. A cooling evaporating gas with low evaporation temperature, or compressed air may also be applied to the tissue or to the surfaces in contact with the tissue. A solenoid valve may be used to control the amount, duration, and timing of the ejected gas (for example a environmentally compatible, Freon—like cooling gas). Such a gas may be applied before during or after the application of treatment energy. If the tissue is cooled by an evaporative fluid prior to the application of energy it may be cooled by a fluid at about temperature of about −25 degree C. to about −100 degree C., but the fluid application time (burst time) must be limited to less than about 100 ms and at the lower temperature range described above to less then about 50 ms. If the evaporative fluid temperature is higher than about −25 degree C. and is as high as about 0 degree C., longer coolant fluid burst may be used, for example, from about 100 ms to about 200 ms. In Some embodiment the temperature of the cooling fluid may be below −100 Degree C. or from about 100 degree C. and about 0 Degree C., or from about 0 degree C. and about 45 Degree C., or from about 25 Degree C. to about 45 Degree C.

Additionally or alternatively, cooling of the tissue can be achieved by contact with the mechanical force or stress delivering member, for example a metal hummer head, or metal piston, or other members of the device which come in contact with the tissue which can, for example, be made of high thermal conductivity sapphire (since sapphire is transparent to some EM energy, it can be used to both cool the tissue, deliver treatment energy, and be used a stress or force delivering member).

Materials with suitable for thermal energy delivery and removal, may have thermal conductivity of about 0.1 W/(Cm*K) or higher, or, in some embodiments, may have thermal conductivity of about 0.5 W/(Cm*K) or higher.

6) A Control and Feedback Member to Control and Synchronize and Adjust Operation.

The devices and methods may also comprise a feedback member and control member. A feedback member may comprise, an OCT, Optical feedback, US sensors and optical feedback, electrical feedback, Optical feedback, Microscope and magnifying lens feedback. Additionally or alternatively a heat sensing device may provide information about the temperature of the tissue. For example, a thermocouple, or an IR camera, or an IR detector, may monitor treated tissue temperature and may be couple to a controller to control the treatment energy, the mechanical force, stress, strain or pressure applying member, or to modify and adjust the cooling rate (increase energy removal rate, decrease it, or leave thermal energy removal rate, according to the feedback from the thermal sensing device).

Additional sensor may monitor acoustic and mechanical characteristics of the tissue, for example, without limitation, stress, strain, pressure, suction, vacuum, rarefication, or stretching of the treated tissue. Again, such sensors may provide said information to a controller that may, according to such communicated information, change the application rate of treatment energy, mechanical force or stress applicator member, cooling member, or other components and or steps used by the device and/or method of the present invention.

7) Heat Delivery or Heat Removal (Cooling)

The devices and methods may also comprise a cooling or heating members, for example, a Thermoelectric cooler (TEC), Conduction cooling, evaporative cooling or heating, conduction heating, convective cooling or convective heating (e.g. a circulating fluid in a sapphire window in contact with the tissue). A reversal of polarity in the TEC and/or, placement of heaters, or heated circulating fluid or liquid, or heating with energy from irradiative source, may also accomplish heat delivery.

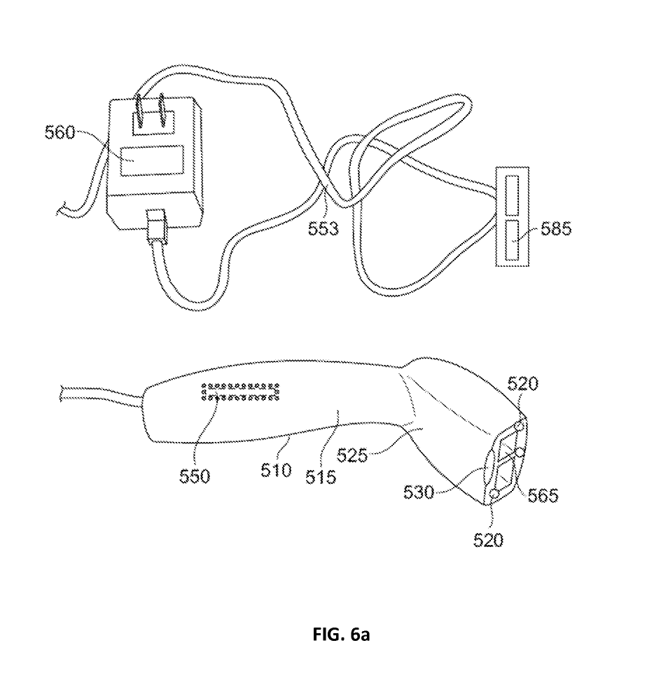

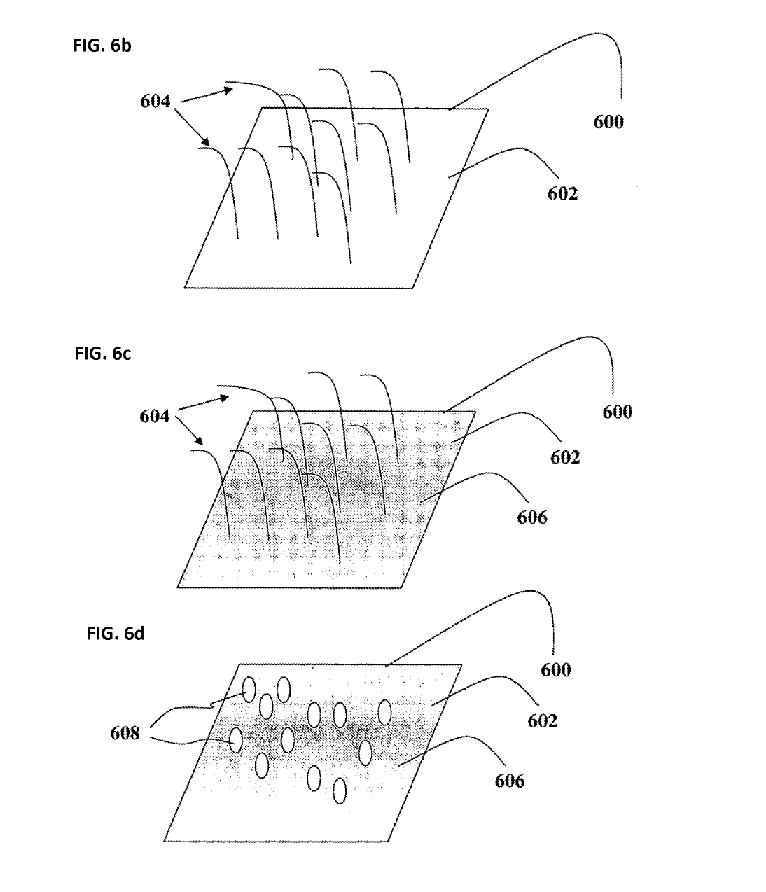

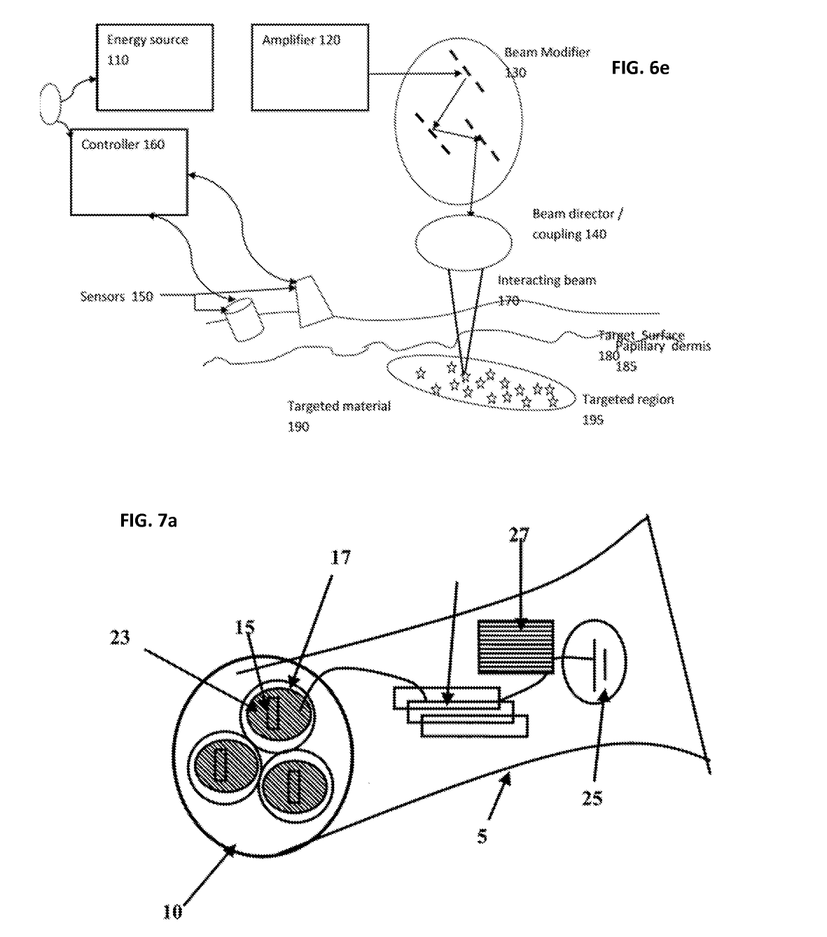

FIG. 6a shows another embodiment showing the possible composition of a device for treating Acne and various other skin conditions. The encasing 510 can be made of plastic or metal or other suitable materials and has a handle 515, for example with an approximate diameter of from about 1 cm to about 5 cm and preferably about 2.5 cm in diameter. The handle can also contain a wire connection 555 to a wall plug or a transformer 560 as shown, or a battery 550. The handle may also contain a control board, an off on switch, and capacitors. The treatment head 520 can also be made of plastic or metal material, or other suitable materials The treatment head has treatment windows 565 as discussed above and may also contain LED sources 520 with appropriate wavelength for example for wound healing, bio-stimulation, reduction of acne bacteria, sterilization, skin and collagen rejuvenation, or reduction of pigmented lesions. The treatment head 525 may also comprise a laser source 530 for cosmetic and skin treatments. With appropriate selection of wavelength and intensity and optical diffraction elements such a laser source can be used to treat acne, skin rejuvenation, wrinkle reduction, pigmented lesion and discoloration and reduce the presence of hair on the skin. An opto-thermal converter element 585 may be attached or swung or placed to the front of the treatment head 525 in front of the treating window 565 to provide a surface thermal interaction by converting light from the treatment windows 565 to heat by use of the converter 1310 and 1320 (see FIG. 3a) with substance capable of partial or complete absorption of the light from the plurality of light sources.

FIGS. 6B-6D illustrate yet another method for treating the hair. As depicted in FIG. 6B, the hair region 600 of the skin 602 covered with the targeted hair 604 is allowed to grow to a sufficiently long level (for example, from about 2 mm to about 50 mm and preferably 3 mm to 20 mm). As depicted in FIG. 6C, the hair 604 and skin 602 are covered with reflective coating material 606 that is biocompatible and does not damage or irritate the skin 602 in any way. Next, once the coating 606 is sufficiently dry, the hair shafts 604 are pulled out of the skin 602 (for example by means of waxing, such as traditional wax material). Preferably, the reflective coating material 606 is such that it adheres to hair wax and so when the hair shafts 604 are pulled out some of the reflective material 606 is pulled out with them. As depicted in FIG. 6D, the pattern left behind after the hair 604 is pulled out is that of a skin 602 covered with reflective coating 606 with regions 608 that are not covered with non-reflective coating 606 around the former locations of the (now-removed) hair shafts. Next, the skin 602 is irradiated with an energy source that is reflected by the reflective coating 606, but is absorbed by the regions of the skin 602 exposed by the removal of the hair shafts and of some of the reflective coating 606 around the former locations of the (now-removed) hair shafts. Such reflective material may comprise a transparent cream such as for example a KY-Jelly or Agar embedded with high concentration of reflective aluminum particles (or other reflective metallic particles), the density and size of said reflective aluminum or metallic particle is large and high enough so that they reflective substantially most of the light or electromagnetic radiation back and away from the bulk of the skin. For example, in an embodiment the reflective material may be made of a transparent cream or agar for example, a KY Jelly) with aluminum particle ranging in size from about 1 micrometer to about 5 mm and preferably form about 50 micrometer to about 1 mm in size. The particle density (number per unit volume of said reflective material) should be such that from about 80% to about 99.9% of the incoming radiation if reflected back away from the surface of the skin.

Optionally, a cooling is applied to the skin before, during or after the removal of the hair shaft with the reflective coating. The cooling action protects the top layer of the skin coated or not, but allows transmittance of the energy through the opening into deeper layers of the skin and preferably into the region of the skin around the hair shafts in the deeper skin around the hair roots and other components of the hair responsible for hair growth. FIG. 6B represents the hair covered pre-treated skin 602 with hair 604 that was allowed to grow as described above. FIG. 6C represents the skin 602 with hair 604 that was allowed to grow as described above and the skin 602 is covered with reflective coating material 606. FIG. 6D shows the skin 602 that was covered with reflective material 606, but the hair 604 was removed, leaving behind region 608 of absorbing location around the hair follicles, while most of the rest of the skin 602 is still covered with hair reflective coating 606.

Such a method can also utilize a corresponding device comprising an element capable of dispensing a material with a reflective ability capable of reflecting the incoming energy. The device will also comprise an element capable of selectively contacting the hair on the skin. The element is also capable of pulling the hair shafts out, thus removing at least some of the reflective material around the hair follicles without reflective coat. Next, the device will apply energy to the skin; the energy is capable of penetrating the skin through portion that lacks the reflective coating, but the energy is otherwise reflected from the regions of the skin coated with reflective substance.

FIG. 6e illustrate an embodiment of the principle of device and method for tattoo removal. A device for tattoo removal, comprise, for example, of an energy source, 110, an optional amplifier, 120, a beam modifying member 130 a coupling/directing member, 140 an optional sensor member, 150, a controller 160.

A method for removing tattoos, said method comprising the device of FIG. 6, wherein an output beam 170 is directed towards the skin surface 180, and is focused, either spatially or temporally or both onto the tattoo particles 190.

The method further comprises manipulating the energy beam 175 emerging from the energy source 125 so that the energy of the pulses arriving at the targeted region 195 is such that volumetric power density is above the volumetric power density for photodisruption of the targeted substance, for example, tattoo ink particles.

The method farther comprises a observing at least some interactions from some pulses and monitoring characteristics of said interaction with said targeted substance 190, delivering said sensor 150 information to the controller 160 for processing, and deciding (Manually, or automatically) if said targeted substance should be removed or not. If after a said sensor data is examined and said targeted substance that was interacted with is determined not to be a substance that the use want to remove, then the beam 170 is blocked, or the energy source 125 is stopped so that interaction with said targeted substance is stopped until further decision is made by the operator.

Another embodiment of the method and device comprises using a LIBS detector to identify the targeted material to be removed. For example, in the case of removal or reduction, or interaction with tattoo ink particles, said ink particles has chemical composition that is distinct from the skin tissue. Spectroscopic detection of said targeted tattoos material can be used to easily distinguish between skin tissue and that of the targeted tattoo inks

Similarly, the acoustic and recoil or other mechanical characteristics of the interaction may be different when said interacting energy pulses are interacting with the targeted substance as oppose to interaction with the surrounding skin.

In one embodiment, for example, a short or more preferably an ultrashort pulse of about 100 ns or less or even about 10 ns or less or even about 1 ns or less or more preferably about 100 ps or less or about 10 ps or less is directed towards a targeted towards a targeted region. The Beam is directed by the member 140 towards the targeted region and its focus position is changed (raised or lower) until an interaction is with the targeted substance 190 is observed by the sensors 150. The interacting beam is moved by the beam director/coupler 140 along the interaction region. The sensors 150 monitor the interaction characteristics, for example, the interaction emission spectra. Interaction emission spectra will be different for different targeted substance. For example, observe the interaction emission spectra for bone versus spinal cord material, shown in figure TAT2. Similarly, the emission from native skin tissue will be different than emission from ink substance (see table TAT1, for chemical composition of skin and tattoo inks).

So it becomes relatively easy to see, that LIBS, or Luminescence emission breakdown spectroscopy is a useful method to aid in the monitoring of the interaction and ensuring that substantially targeted region or acted upon and when the emissions from said targeted substances, for example, India ink or other tattoo material is replaced by emission form native skin tissue, the interacting beam 170 should be redirected to other areas of interest.

One of the sensors, as was pointed above can be a sensor of mechanical disturbance, for example a transducer, which will monitor the shock wave or Acousto-mechnical disturbances from the targeted ablated or photodisrupted or otherwise modified targeted volume.

Additionally or alternatively a sensor can also be a camera, for example a CCD camera that feed its information to the controller 160. The controller, for example, a computer or processor, then directs (or allow the operator to direct) the interacting beam 170 towards the general area of the targeted region. Additionally or alternatively, in a further embodiment, the feedback from the camera sensor allow keeping the interacting beam in the general vicinity of the targeted volume 195 where the targeted substance reside, (for example the area of the tattoo ink) and the LIBS sensor 150 monitor the actual type of ablated material that is interacting with the incident beam 170. When targeted substance is substantially eliminated or modified, or the beam exit the targeted region 195 the LIBS feedback allow the computer (Or manually to the operator) to redirect the beam 195 back to the targeted volume, or to stop the operation, or to direct the beam 195 towards deeper or shallower layers.

cut cells vaporize 40 um particle average sizes 250 um to 1700 um ink particle size 1-2 um 40 um 640000 layer by layer

Electronic Flash Units, Heat Sources, and Energy Sources

In another embodiment, the system comprises an electronic flash units (often called photographic strobes) which based on the same principles of operation whether of the subminiature variety in a disposable pocket camera, high quality 35 mm camera, compact separate hot shoe mounted unit, or the high power high performance unit found in a photo studio ‘speed light’. All of these use the triggered discharge of an energy storage capacitor through a special flashtube filled with xenon gas at low pressure to produce a very short burst of high intensity white light. Such bursts are often on the order of a millisecond. In one embodiment, a pulse duration from about 0.01 ms to about 1 seconds and preferably from about 0.3 ms to about 0.3 seconds and most often on the order of about 1 ms to about 100 ms.

The typical electronic flash comprises of four parts: (1) power supply, (2) energy storage capacitor, (3) trigger circuit, and (4) flashtube.

An electronic flash works as follows:

1. The energy storage capacitor connected across the flashtube is charged from a 300V (typical) power supply. This is either a battery or AC adapter operated inverter (pocket cameras and compact strobes) or an AC line operated supply using a power transformer or voltage doubler or tripler (high performance studio ‘speed’ lights). These are large electrolytic capacitors (100 to 1000+uF at 300+V) designed specifically for the rapid discharge needs of photoflash applications. Such rapid discharge is suitable because the device converts such optical discharge into thermal energy at the surface of the skin. Such rapid deposition allows determination of a known quanta of energy to be deposited on the surface of the skin and a known short deposition time. These two elements prevent excess energy from diffusion into deeper tissue area and unwanted collateral damage.

2. A ‘ready light’ indicates when the capacitor is fully charged. Most monitor the voltage on the energy storage capacitor. However, some detect that the inverter or power supply load has decreased indicating full charge.

3. Normally, the flashtube remains non-conductive even when the capacitor is fully charged.

4. A separate small capacitor (e.g., 0.1 uF) is charged from the same power supply to generate a trigger pulse.

5. Contacts on the device shutter close at the instant the shutter is fully open. These cause the charge on the trigger capacitor to be dumped into the primary of a pulse transformer, whose secondary is connected to a wire, strip, or the metal reflector in close proximity to the flashtube.

6. The pulse generated by this trigger (typically around 4-10 KV depending on the size of the unit) is enough to ionize the xenon gas inside the flashtube.

7. The xenon gas suddenly becomes a low resistance and the energy storage capacitor discharges through the flashtube resulting in a short duration brilliant white light.

The energy of each flash is roughly equal to ½*C*V² in watt-seconds (W-s) where V is the value of the energy storage capacitor's voltage and C is its capacitance. Not quite all of the energy in the capacitor is used but it is very close. The energy storage capacitor for pocket cameras is typically 100 to 400 uF at 330 V (charged to 300 V) with a typical flash energy of 10 W-s. For high power strobes, 1000 s of uF at higher voltages are common with maximum flash energies of 100 W-s or more. Another important difference is in the cycle time. For some battery operated devices, it may be several seconds—or much longer as the batteries run down. Larger devices or transformer-powered devices, the speed can be a fraction of second cycle times which are common.

In some embodiments the user, usually a skin care professional or a physician, may want to be able to heat up the skin epidermis AND dermis, beyond collagen denaturation temperature. In such cases, a rapid succession of light pulses may be desired. Here, a common camera feature may be used in such cases. For example, the red-eye reduction feature provides a means of providing a flash twice in rapid succession.

A variety of repetition rates may be used depending on the needs. For consumer use, a slower repetition rate is contemplated to avoid pulse-to-pulse thermal build up. However, in professional or physicians use a higher repletion rate is contemplated, to allow, for example, sufficient energy build up in the target tissue so that so that the epidermis is heated for example to a depth of mid reticular dermis and to a time duration that results in permanent denaturation of the collagen, to allow skin rejuvenation and wrinkle reduction.

In this embodiment, the main flash would require sub-second recycle time which is not a problem if an energy conserving flash is used. However, it would add significant additional expense otherwise (as is the case with most cameras with built in electronic flash). A separate little bulb is effective and much cheaper.

In another embodiment, an automatic exposure control electronic flash units may be used. Here, automatic electronic flash units provide an optical feedback mechanism to sense the amount of light actually reaching the targeted tissue. The flash is then aborted in mid stride once the proper exposure has been made. This means that the flash duration will differ depending on exposure—typically from 1 ms at full power to 20 microsecond or less at lower power levels.

The device and a method for treating a target surface, in particular a skin surface, and the condition of acne, comprising the steps of a) activating an energy source, b) bringing an energy transporter element into contact with the energy source, c) allowing said energy transporter element to absorb some of the energy from the energy source, d) disconnecting said energy transporter and moving it into contact with a target surface, e) allowing a predetermined amount energy from said energy transporter to be transferred to the target surface so that a desired effect is achieved. The method further envisions that the target surface is a biological tissue, in particular skin tissue, and the desired effect is a physical, chemical or biological effect.

The method further envisions a desired effect which is a thermal change in the target surface characteristics.

In yet another embodiment, an energy source creates thermal energy deposition on the surface of the skin to alleviate skin conditions. In further elaboration of this effect the thermal energy deposition alleviates acne conditions. It is possible to alleviate such acne condition for example, by creating expansion of the skin surface so that pores and pore openings are enlarged, allowing drainage of puss, sebum and other undesired material, or even the expulsion of black heads.

The device and method described herein also envisions an embodiment wherein the desired effect of the thermal expansion of the skin surface will allow opening of the skin pores so that said expansion allows at least some enhancement of material or substances to be transported across the skin barrier through spacing between various skin components and through the pores in the skin and the skin surface.

### Heat Shuttle

In yet another embodiment, a device for thermal material conditioning is envisioned, wherein said device comprises a heat source which is elevated to the desired temperature and maintained at said desired temperature, means to transporting said thermal energy or heat, such as a heat shuttle in contact with the heat source so that thermal energy can diffuse from the heat source and maintain said heat shuttle at the same temperature as the heat source. The device preferably also includes a trigger that allow an operator to willfully release the heat shuttle from contact with the heat source and bring it into contact with the target treatment area so that thermal energy can flow from the heat shuttle to the targeted treatment material. The device allows said heat shuttle to maintain contact with the targeted treatment area of the target material for a period of time sufficient to bring the target material and the heat shuttle into thermal equilibrium so that substantially no heat flows from the heat shuttle to the targeted material. The heat transporter can then be removed from contact with the skin or other target surface and brought back into contact with the heat source so that it is reloaded with thermal energy.

In yet another embodiment, the transporter of thermal energy or heat shuttle is allowed to maintain contact with the targeted material area for a period of time from about 0.1 microsecond to about 1 second. Similarly, the heat source is allowed to deliver heat to the skin surface for a period of from about 0.1 microsecond to about 1 second.

In another embodiment, the thermal energy source is allowed to deliver a quanta of energy to the surface of the skin in such a way that it brings the skin surface to a temperature of between about 45 degree C. and 500 degree C., preferably, however, the temperature of the surface of the skin reaches between about 50 degree C. and about 350 degree C.

In one embodiment, wherein the device will bring the target material surface (preferably the a skin surface) to a temperature that results in expansion of the skin surface and wherein said expansion of the skin surface will result in at least a 1 micrometer expansion of the pore diameter size. In another embodiment, the steps described above of a device or a method for treating skin conditions utilize an energy source which loads an energy transporter with thermal energy (and increases said transporter's temperature to a desired temperature) then bring said energy transporter into contact with the skin so that the thermal energy may be deposited within the skin (with a desired time duration and desired amount of energy transported within said time duration), this is repeated multiple times at a repetition rate of between about 0.1 Hz and about 1 KHz and preferably between 0.2 Hz and 10 Hz.

In further embodiments, the device and method described about envision utilizing electrical energy as an energy source. Further embodiment envisions the energy source as a thermal energy source wherein the heat source is a thermo-electric cooler. Further embodiment envisions the energy transporter as a heat shuttle made of metal. Said metal heat transporter may also be made of a thin metal sheet of between about 1 micrometer in thickness and about 10 mm in thickness and preferably between about 70 micrometer and 400 micrometer.

Alternatively, and in an embodiment, said heat source is an electric energy source, for example, an electric wall outlet, an electric wall outlet with a transformer, a battery, or a battery and capacitor combination, wherein said electrical energy is brought through the energy transporter (for example, electric wires or metal plates) into contact with a the target surface or skin, where they deposit energy in the form of thermal energy. For example, a metal electric resistor or most materials with inherent electrical resistance may serve for such a purpose. Additionally, a thermoelectric cooler may serve to convert electrical energy into heat with the added benefit of being easily switchable to cooling the target surface after the thermal energy deposition phase. Preferably said electric energy is pulsed so that electric energy, which is then converted to thermal energy which is deposited into the skin surface, is also pulsed. Such pulsed energy deposition phase should last between about 0.1 microsecond and about 100 seconds and preferably, between about 1 ms and about 1 seconds.

In a further embodiment, a device for skin conditioning comprises a heat source wherein a heat shuttle makes contact with said heat source, a console containing both the heat source and the heat shuttle, a transfer compartment capable of separating the heat shuttle from the heat source. The treatment process includes transferring the heat shuttle into contact with the target material, keeping the heat shuttle in contact with said target material for a predetermined period of time, and then removing the heat shuttle from the target material and transferring it back into contact with the heat source. The process can then be repeated multiple times.

A device is capable of repeatedly and automatically heating a target material by bringing an movable component into contact with a high temperature source, by keeping the heat shuttle in contact with a heat source, a. moving the heat shuttle away from the heat source and into contact with a target material to be heated; b. maintaining contact between the heat shuttle and the target material for a predetermined length of time; c. removing the heat shuttle from the target material and bringing it back into contact with the heat source and repeating said steps for a predetermined period of time or a predetermined number of repetitions. In some embodiments the device interacts with a target material which is the skin.

The device of the embodiment further envisions bringing the heat transporter into contact with the skin target material for a sufficiently long time to allow expansion of the skin so that at least one skin pore expands and opens enough to allow enhanced material transport through said at least one skin pore. Alternatively, the heat from the source is allowed to be transferred into the skin for a limited amount of time, sufficient to deposit enough thermal energy into the skin allow expansion of the skin so that at least one skin pore expands and opens enough to allow enhanced material transport through said at least one skin pore. In this embodiment the thermal energy source can deposit its energy either by direct transport or conduction into the skin or through the action of an intermediate heat transporter.

Further embodiments include a device for treating material conditions comprising, a thermal energy source, a heat shuttle in contact with said heat source said heat shuttle comprises a body capable of loading up with thermal energy and two latches, One latch is connected to a spring which tend to propels the heat shuttle towards the target material and keeps it in contact with said target material, The second heat latch is picked up (hooked to) by a rotating motor which propels the heat shuttle back up and brings it back into contact with the heat source. The latch is constructed with a slop so that the rotating motor eventually slips off it allowing the now compressed spring in constant contact with latch number one to propel the heat shuttle again into the target material. The process is repeated until the operator stops

The above can also be envisioned wherein the role of the spring and the motor is reversed, i.e., the motor is the one pushing the heat shuttle into the target material and the spring tends to drive the heat shuttle away from the target material and into contact with the heat shuttle.

In further embodiment, the device for material conditioning comprises a magazine full of spring loaded individual heat transport elements (much like bullets are packed into a magazine of an automatic machine gun or rifle magazine such as the military M 16 or Uzi submachine gun). The heat shuttle “bullets” comprise at least thin aluminum plate to be loaded with heat energy and two latches. The latches should be made of non-thermally conduction material or at least a discontinuing between metal parts so that said thermal energy remains substantially confined to the heat shuttle. It also includes a spring pushing against one latch in order to allow it to create a good thermal contact with the heat source, a motor driving against the other latch to push the heat shuttle down away from the heat source and into contact with the target material, a remover arm pushing the spent heat shuttles (whose thermal energy was used) away from the device and disposing of them), a loader arm pushing the “bullets’ heat shuttles into place where they can be picked up by the spring loading mechanism and be pushed into contact with the heat source.

A motor is used to drive a piston up against a spring (spring loading mechanism). The spring discharge after a stop at the station that allows it to load up with thermal energy. The shuttle is thus propelled by the spring towards the target material to be treated.

The amount of heat energy that was loaded up into the shuttle is finite, so the amount of heat or thermal energy that is discharged into the target material is finite as well.

The methods and devices described below contemplate incorporating various thermal energy sources to achieve the desired skin surface effect of temporary but biologically significant expansion so that trans-dermal transport is possible and indeed enhanced. To achieve this effect the thermal energy source can be optical, chemical, or electrical. In all embodiments, the source is to produce sufficient amount of energy which is then to be delivered to the skin surface for only a limited amount of time so that no collateral damage is to result, the expansion is temporary and does not result in any burn to the skin and the source of energy flow into the target skin is cut off at the end of a predetermined time interval so that only a predetermined amount of energy is allowed to be deposited into the skin.

Such design of these embodiments in combination of the relatively slow thermal energy diffusion within the skin, allows concentration of sufficient energy in the upper layer of the skin to enhance transport properties but does not allow sufficient amount of energy to penetrate below the epidermal/dermal junction so that substantially the dermis remains free of burns or any undesirable effects.

One such embodiment envisions the use of electric energy as heat source. In this case, the flow of electrons through a substance with inherent resistance results in joule or resistive heating (one such example will be an electric wire, another is a hot soldering iron). A heat shuttle can then be brought into contact with such electric-energy based heat source and then shuttle the energy into contact with the target material. Alternatively, said electric heat source is connected directly with the target material or skin via conducting material that serves to shuttle the heat and electric energy and the source energy is cut off after a predetermined time. For example, the source of energy can be a full charged capacitor that is connected to the skin via conducting transporter (for example metal wires or metal plates), the capacitor is then allowed to discharge its energy into the energy transporter that is in contact with the targeted skin surface.

Further embodiment envisions a method for Material Conditioning comprising of: a heat source brought to a desired temperature and maintained at that temperature, a heat shuttle (HS) maintained at the source temperature through thermal contact with the heat source, means to willfully trigger said heat shuttle (HS) motion so it is released from thermal contact with said heat source and is brought into thermal contact with the targeted treatment area, allowing said heat shuttle to maintain contact with the treatment area for a period of time sufficiently long to transfer sufficient thermal energy to the targeted region to cause thermal expansion of the treated area and bring about the desired effects including the treatment of skin conditions. Removing the HS from contact with the targeted area and bringing it back into thermal contact with the heat source

The method above further contemplates a contact period between the heat shuttle and the treatment area is from about 0.1 ms to about 1 second and preferably from about 1 ms to about 100 ms (In water-like material such a period of 100 ms will allow thermal energy to diffuse to roughly a depth of penetration of about 300 um). The method of further comprises repeating all steps at the repetition rate of between 0.1 Hz and 1 KHz and preferably at a repletion rate of between 0.2 Hz and 10 Hz. In further elaboration of this embodiment, the heat source is powered by electrical heater driven by electrical energy. In yet further possible embodiment, the heat source is a thermo-electric cooling device (TEC) or Paltrier cooling device. Additionally, the heat shuttle can be made of thermally conducting material. In yet another embodiment, the heat shuttle (HS) can be made of metal.

An additional embodiment envisions the heat shuttle as made of metal of sufficient contact area with the target material to allow reasonable work rate and preferably a contact area with the target material of between about 0.2 cm2 and about 4 cm2.

In a further embodiment, the method and device include a heat shuttle made of metal of sufficient volume and heat capacity to allow the heat shuttle to carry thermal energy sufficient to raise the temperature of the upper layers of the skin to cause the desired effect and in particular to improve or cure undesired skin conditions. Additionally the heat shuttle (HS) may be made of thermally conducting material in the form of a sheet with a thickness of between about one micrometer and about one millimeter in thickness and preferably between 70 micrometer and 200 micrometer, so that the desired biological effect is achieved.

For example, in an embodiment the target material is the skin and sufficient thermal energy is delivered by the heat shuttle to the targeted skin to cause thermal expansion of the skin in the treated region and opening of the pores in said skin region to allow substance to flow in or out of at least a portion of the skin through at least some layers of the epidermis.

The device for material conditioning, and in particular for treating skin conditions, comprises: a) A heat source; b) A heat shuttle in contact with said heat source; c) A console to contain both the heat source and the heat shuttle (HS) and to ensure that neither is in thermal contact with the target treatment area during at least part of the device operation time; d) A transfer element capable of separating the heat shuttle from the heat source and bringing it into contact with the target material keeping the heat shuttle, keeping the heat shuttle in contact with said target material for a predetermined period of time then removing the HS from the targeted material and bringing the HS back into thermal contact with the heat source. This device for material conditioning should also be capable of repeatedly and automatically heating a target material by heating a heat shuttle (HS) by keeping it in contact with a heat source, moving said heat shuttle away from the heat source and into contact with the target material, keeping the HS at the target material for a predetermined period of time, removing the HS from the target material and bringing it back into contact with the heat source, repeating said steps at a predetermined repletion rate for a predetermined total operation time period. The device of this embodiment should further comprise keeping the heat shuttle in contact with the target material for a sufficiently long time to allow thermal expansion of the target material.

The device of this embodiment also contemplates that the target material is skin and the heat shuttle is kept in contact with the skin for a sufficiently long time to allow thermal expansion of the skin and opening of the pores in said skin region to allow substance to flow in or out of at least a portion of the skin through at least some layers of the epidermis.

The device for material conditioning is capable of repeatedly and automatically heating a target material by heating a heat shuttle (HS) by keeping it in contact with a heat source and moving said heat shuttle away from the heat source and into contact with the target material, keeping the HS at the target material for a predetermined period of time, removing the HS from the target material and bringing it back into contact with the heat source, repeating said steps at a predetermined repletion rate for a predetermined total operation time period. The present device further contemplates keeping the HS in contact with the target material for a sufficiently long time to allow thermal expansion of the target material.

In further elaboration of this embodiments, the target material is skin and the heat shuttle is kept in contact with the skin for a sufficiently long time to allow thermal expansion of the skin and opening of the pores in said skin region to allow substance to flow in or out of at least a portion of the skin through at least some layers of the epidermis. The device further comprises a pump to lower the pressure within the device chamber and create a tighter seal to the skin. This will allow: better contact with the skin, removal of debris from the skin and pores, and reduction of the amount of air within the chamber in order to minimize heat conduction and heat removal from the HS during it passage from the heat source to the targeted skin. This embodiment further envisions the device comprising generating lower pressure through a pump.

The coating the heat shuttle in the above embodiments with nutrients, drugs, medications or any other substance is desirable to deliver into the target surface. Furthermore, the device of any of the above embodiments contemplate such nutrients, medications, or drugs or any other substance is applied to the same area of the skin before, during, or after the action of the heat shuttle. The device of any of the above embodiments, wherein, a container and dispenser containing and dispensing a drug or any other substance that one wishes to deliver into the target surface is attached to the heat shuttle apparatus and delivery a desirable substance before, during or after the action and passage of the heat shuttle.

Alternatively, in another embodiment, CW lamps such as tungsten lamps 840 may be embedded in the foot plated to allow heating of the skin to just under the threshold for hair damage. The flash lamp 830 may then be used to bring the hair matrix cell and possibly the papilla to above threshold for damage or threshold for retardation of normal growth rate. This will result in damage to hair follicle leading to reduced hair density and or reduce hair shaft size.

In yet another embodiment the heating elements 840 are made of thin film resistors and ultrasound heaters. Alternatively the heating elements 840 may be made of mono-polar or bipolar heads or microwave or radiation emitting heads.

Another method utilize bulk heating of the skin volume containing the hair follicle in combination with the action of energy from a secondary energy source, preferably the secondary energy source is a low power light source, and preferably a broad band lamp such as the one used in 2004-2006 by the Fuji or Kodak single use camera or by the par Perkins Elmer lamps described below. The same circuitry utilized by the Kodak or Fuji single use cameras, or digital cameras, or the circuitry described below, may be utilized. In this method or version of the device, the first energy source raises the temperature of the follicle to close to the one capable of disrupting growth the hair follicle and the second energy source raises it above said level needed to disrupt growth of the hair follicle. The first energy source can be for example, a continuously operating (CW) lamp, an electric heater, a microwave heater, a sound energy source capable of heating the tissue (e.g. ultrasound energy source) or any other energy source. A tanning lamp, a CW laser, an LED source or plurality of LED sources, a warm bath or warm towel or other source capable of heating the skin tissue to the full length of the embedded hair follicles, (i.e. the relaxation time corresponding to the thickness of the dermis).

In another embodiment, a device similar to the flash lamp devices described earlier contains a more powerful broad band lamp capable of raising the temperature of the skin to a level and time duration from about 0.5 seconds to about 5 seconds. A second flash lamp is then fired within the thermal relaxation time of the targeted skin volume

The second pulse of light is then capable of raising the temperature of the damage threshold for the hair or above the hair cells damage threshold, or threshold for damage to the components of the hair support system (blood vessels or nutrient carrying elements which sustain the hair follicle). Alternatively the second pulse raising the temperature of the hair and its immediate surrounding region to a level that retard or decrease hair growth such that the hair follicles in the treated area grow less rapidly, less fully, to a thinner dimension, or otherwise to a less extent in density, appearance, or strength, or otherwise adversely affect hair growth.

Alternative, a single flash lamp can be used with an IGBT pulse-forming circuitry such as in the “Redeye reduction” digital camera flash or other “smart” camera flashes that fire sequential pulses. An example of such IGBT circuitry is given by the Toshiba IGBT GT8G132.

Alternatively, an electrical heater such as resistive heating or thin film resistors heats the surface of the skin, allowing the skin to reach an elevated temperature, but one that is below the permanent damage threshold to the skin, then a sequential burst of energy that is selectively absorbed by the hair follicles or their immediate surrounding elevated the temperature of the hair follicle and its surrounding so that the second pulse raises the temperature of the hair and its immediate surrounding region to a level that retard or decrease hair growth such that the hair follicles in the treated area grow less rapidly, less fully, to a thinner dimension, or otherwise to a less extent in density, appearance, or strength, or otherwise adversely affect hair growth.

Alternatively, many such plurality energy sources can be used with resistive heating or electrical heater with utilizing electrical resistors to heat the surface of the skin and allow the skin to reach an elevated temperature that is below the permanent damage threshold to the skin, then a sequential burst of energy selectively absorbed by the hair follicles or their immediate surrounding elevates the temperature of the hair follicle and its surrounding so that the second pulse raises the temperature of the hair and its immediate surrounding region to a level that retards or decreases hair growth such that the hair follicles in the treated area grow less rapidly, less fully, to a thinner dimension, or otherwise to a less extent in density, appearance, or strength, or otherwise adversely affect hair growth.

Such energy source combinations are summarized by the following Table 2.

TABLE 2

| Primary energy source | Sequential energy source (or Secondary source |
|---|---|
| RF, | Light |
| Microwave, | Lasers |
| Electrical heater | Flash lamp |
| Infrared | EM Radiation |
| Near infrared | Electromagnetic (EM) energy with skin |
| Ultrasound | penetration ability but selective absorption in |
| Electrical heating | the hair follicle or the hair follicle immediate |
| Chemical | surroundings. |
| Mechanical energy | Electromagnetic (EM) energy with skin |
| Mechanical heating | penetration ability but such that are absorbed |
| Chemical heating | by an adjunct substance that is inserted into the |
| Light | vicinity of the hair follicle and in particular the |
| Laser | vicinity of the hair root, papilla and matrix |
| Flash lamp | cells. |
| EM radiation | RF, |
| Plasma energy | Microwave, |
| Pulsed electrical | Electrical heater |
| heating | Infrared |
| Thermo-electric devices | Near infrared |
| Peltier devices | Ultrasound |
| | Electrical heating |
| | Thermo-electric devices |
| | Peltier devices |

The energy or light source, 1020 may emit continuous energy (CW) energy, or CW Light output, or CW laser beam), or the energy or light source 1020 may emit pulse energy. If pulsed the pulse energy may be of a pulse duration ranging from About 3 fs to about 100 minutes, From about 3 fs to about 10 minutes, From about 3 fs to about 1 minute, From about 3 fs to about 5 minutes, From about 3 fs to about 100 ms, From about 3 fs to about 10 ms, From about 3 fs to about 1 ms, From about 3 fs to about 100 microseconds (us), From about 3 fs to about 10 us, Form about 3 fs to about 1 us, From about 3 fs to about 100 ns, Form about 3 fs to about 10 ns, From about 3 fs to about 1 ns, From about 3 fs to about 100 ps, From about 3 fs to about 10 ps, From about 3 fs to about 1 ps, From about 3 fs to about 100 fs, From about 3 fs to about 10 fs, From about 1 ms to about 5 minutes.

The pulse repetition rate of the light or energy emission may or may not be synchronized with the mechanical energy impact repetition rate. If said light or energy emission is synchronized it may have the following pulse repetition rates (or frequencies): about 0.001 Hz to about 1 GHz, about 0.01 Hz to about 100 MHz, about 0.1 Hz to 1 about 0 MHz, About 0.1 Hz to about 1 MHz, about A single shot, About 0.1 Hz to about 100 KHz, about One Hz to about 50 KHz, about One Hz to about 10 KHz, about One Hz to about 5 KHz, about One Hz to about 3 KHz, about One Hz to about 1 KHz, about 3 Hz to about 3 KHz.

Mechanical Energy Transfer and Massaging Components

The treatment Head may comprises a mass capable of delivering mechanical Impact to the treatment area or targeted media. Mechanical impact is defined as force times the duration of the application of the force:

I=F*dt=M*dV

Where I is the mechanical impact applied to the tissue by the treatment head, F is the force applied by the treatment head, dt is the time duration during which the force is applied to the targeted tissue or targeted material, M is the mass of the of the treatment head, and dV is the change in velocity of the treatment head due to its collision with the targeted tissue or targeted material.

So the larger the mass of the treatment head and the faster said mass, M, is hitting the targeted region or targeted tissue or targeted area or targeted media, the larger the impact given by the product of the force F and the time said force is applied to the targeted region or targeted tissue or targeted medial

A targeted media, targeted tissue, targeted area, targeted volume and similar expression as the Treatment Zone or Targeted Zone (TZ). The Targeted Zone or Treatment Zone (TZ) thus refer to the region of the tissue or targeted material that is treated in a single stroke of the treatment head.

The targeted treatment region, is the entire organ or material body that is covered in a single treatment session, for example, the whole front of an upper leg may be considered a Targeted treatment region (TTR).

Another way to look at the effect of the Mechanical force F, and Mechanical Impact I:

I=F*dt=M*dV

Is to look at the transfer of mechanical energy from the treatment head (TH) to the targeted TZ by each strike of the TH at the TZ.

The energy contained in the mechanical TH of Mass, M, is according to Newtonian mechanics, the kinetic energy (KE) of the mass prior to heating the TZ

KE=½MV²

Where M is the TH Mass and V is the velocity of the Mass just before it hit the TZ.

This energy is then substantially transferred to the TZ and in the case of tissue the targeted tissue.

For the purpose of the treatment, the TH mass should be from about 10 g to about 100 Kg, and more preferably, from about 100 g to about 20 Kg, and more preferably from about 300 g to about 10 Kg, or from about 500 g to about 10 Kg, or from about 500 g to about 7 Kg, or from about lkg to about 5 Kg, or from about 1 Kg to about 3 Kg.

The TH may further comprise a rectangular base or a circular base.

The TH lower surface, the surface that comes in contact with the TZ, which I shall refer to herein as the Contact Surface (CS) of the TH, may comprises, without limitation, smooth surface, a rough surface, corrugated surface, rough surface with a periodic pattern, rough surface with a predetermined pattern, a rough surface with a random pattern, or a combination of the above surfaces.

The CS of the TH may also have a suction ring or flange connected to a vacuum source so that say treatment head moves within the perimeters of a suction ring or a vacuum enclosure.

The suction ring may be detached form the TH so that the treatment head can be moved up and down within it, and the Suction Ring may provide suction force, or vacuum action, or negative pressure so as to substantially pull the tissue or TZ, or muscle, or Targeted material, up by the force of the suction and towards the impact area of the TH.

If a massager is incorporated into the device, the massaging effect will enhance the delivery of substances into the hair shafts for both therapeutic effects on the skin as well as possibly allowing a substance with thermally conducting properties to penetrate the pores and enhance thermal energy delivery into the lower portion of the hair follicle thus allowing influence on the papilla and hair follicle matrix cells to retard or eliminate hair growth. Such a massager can be constructed from a device capable of generating mechanical vibrations. Alternatively the massager can be made utilizing ultrasound or subsonic energy sources to create vibration and heating on the surface of the skin.

The device of the above embodiment can also be constructed wherein the energy source and massager or substance driver is a thermal element capable of heating the skin to a predetermined temperature range and a predetermined range of lengths of time. A thermal element can create a period thermal expansion in the skin, thus allowing an operator to drive a substance into the skin and in fact create controlled vibration like a massager. For example, bulk heating of the volume of the skin with an energy density from about 0.2 J/cm3 to about 100,000 J/cm3 and preferably from about 0.3 J/cm3 to about 30,000 J/cm2. Exposure range from about 10 microsecond to about 15 seconds and preferably form about 0.1 ms to 500 ms, and wavelength range from about 300 nm to about 1500 nm and preferably from about 400 nm to about 1300 nm.

Further embodiment envisions the device above that can also be constructed wherein at least some of the absorbing substance is allowed to remain on the surface of the skin and is not removed from the skin. The device wherein the massager is an instrument capable of generating using an energy source made of an opto-thermal element. This opto-thermal energy source or driver element may use.

In a further embodiment, the method discussed above is capable of driving an absorbing substance into the hair follicle to enhance light energy coupling into the follicle, the absorbing substance is driven into the skin by thermal means. Similarly, the absorbing substance can be driven into the skin by placing a high absorbing film in contact with the skin and illuminating the high absorbing substance with a light source. Similarly the absorbing substance is driven into the skin by heating the skin area to a predetermined temperature range and in a predetermined time duration.

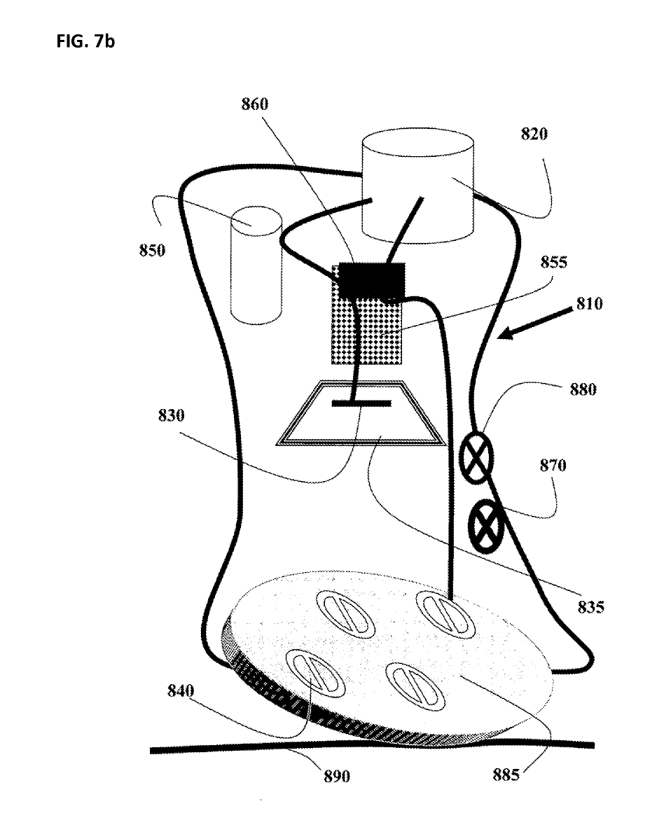

FIG. 7a illustrates the general configuration of a light-based device for skin rejuvenation. A plurality of flash lamps 15 are placed at the treating end (treatment head) 10 of a handheld device 5. The treatment heads deliver a predetermined amount of optical energy. The amount of energy is determined by the discharge energy of a plurality of capacitors 20 powered by an energy source 25, such as a plurality of batteries or any other energy source 25. Each flash lamp 15 is placed inside a reflector 17 and its optical energy is absorbed and at least partially converted to thermal energy by a film 23 of high absorbing substance capable of absorbing said optical radiation. In another embodiment illustrated by FIG. 7a, said flash lamps 15 can be fired sequentially to provide a staggered treatment of different area in a desired predetermined sequence.

FIG. 7b shows another embodiment. A combination of two lamps can be envisioned. In this case, the device contains two energy sources synchronized together. The first energy source is designed to heat the bulk tissue, for example the skin down to the bottom of the dermis, where the hair roots and hair papilla are located. The temperature of the skin is raised, but not above the level sufficient to damage the skin or any living cells within the skin. Since the depth of the dermis can range from about 0.5 mm or less to as many as 5 mm deep (depending on the location of the skin), the diffusion of heat out of that region is not large during a time duration of about 0.5 second to 5 seconds. The second energy pulse is provided by an energy source with less power and with energy characteristics that allow it to be absorbed selectively by the pigments in the skin target and in particular by the pigment in the hair shafts. Thus the second pulse, fired with a duration between about 0.01 second and about 5 seconds and, preferably, between about 0.1 second and about 2 seconds after the first pulse of energy, provides for a follicle damaging “killer”, or growth retarding pulse, that allows retarding effect on hair growth. For example, considering the apparatus 810 of FIG. 7b: A power source 820, for example, a battery, a transformer, an AC adaptor, or a power line, is used to power a first energy source (for example one or more light sources, and preferably a pulsed light source) 830, as well as a second energy source 840, for example one or more electric heaters to be brought in contact with the target skin 890. Preferably, a plurality of pulsed electrical heaters are used as the second energy source. For example, a plurality of capacitors 850 may be used to store electrical energy and discharge the electrical energy in a pulsed manner to both the first and the second energy sources. Alternatively, the second energy source, for example, a plurality of electric heaters may be on in a continuous or semi-continuous manner, or for a longer duration of time, for example, time duration from about a few millisecond to about few minutes and, preferably, from about 100 ms to about 4-7 seconds. The first energy source may then be synchronized to fire after the second energy source has raised the temperature of the target material or skin 890 to a sufficient level just under the damage or control of hair growth. The synchronization of the two energy sources (or possibly more than two energy sources) as well as the control and electronic operation of the device is controlled through an IC circuits board 855. The IC board may also be programmable to various parameters of operation (for example, varying energy discharge level, time duration, and delay time between second and first power energy sources). Such programming may be achieved with an EPROM chip 860.

Finally, as shown in FIG. 7b, willfully charging and triggering of the device operation is provided by the switches 870, and 880 respectively. For example, a base plate 885 in contact with the targeted skin may be embedded with electrical heaters 840. Such electrical heaters, 840, may be made of copper wires shown in FIG. 7b, and the base plate may be substantially transparent so that the light from the lamp 830 can substantially transverse the base plate 885 and reach the skin. The electric heaters, for example, may be turned on continuously and brought to a temperature of, for example, between about 40 degrees centigrade and about 60 degrees centigrade and preferably from about 50 degree C. to about 57 degrees C. The flash lamp 830, may be turned on for a short time duration, for example, from about 0.1 ms to about 900 ms and preferably from about 0.1 ms to about 400 ms, with sufficient energy density to bring the matrix cell of the hair and possibly the region of the hair papilla and hair bulb including possibly the feeding vascular network sustaining the hair follicle, to a temperature above the denaturation threshold of the said matrix cells and said tissue, or to the level above the threshold for disruption of normal vital function of the said matrix cells and said tissue. The reflectors 835 helps redirect the flash lamp 830 light towards the foot plate and into the targeted skin 890. The flash lamp fluence may range from about 0.01 J/cm2 to about 100 J/cm2 and preferably from about 0.1 J/cm2 to about 10 J/cm2.

In yet another embodiment shown in FIG. 1a, an auxiliary cooling component 1252 is activated between 0.1 ms and 1 seconds and preferably from about 1 ms to about 100 ms after the light is discharge thus allowing heat flow to the reach the dermis yet spare the epidermis from damage. The cooling component comprise a container 1252 which is used to contain a cooling agent such as, for example, a gas with low evaporating temperature such as an environmentally compatible Freon-like fluid. The cooling fluid is transported by a tube 1253 or other means to conduct fluid to a discharge nozzle 1254. The nozzle allow controlled timing of the discharge of the cooling liquid that is directed towards the target to remove heat form the target while evaporating. The discharge control can be achieved, for example, with an electronic fuel injection valve which is well known in the art.

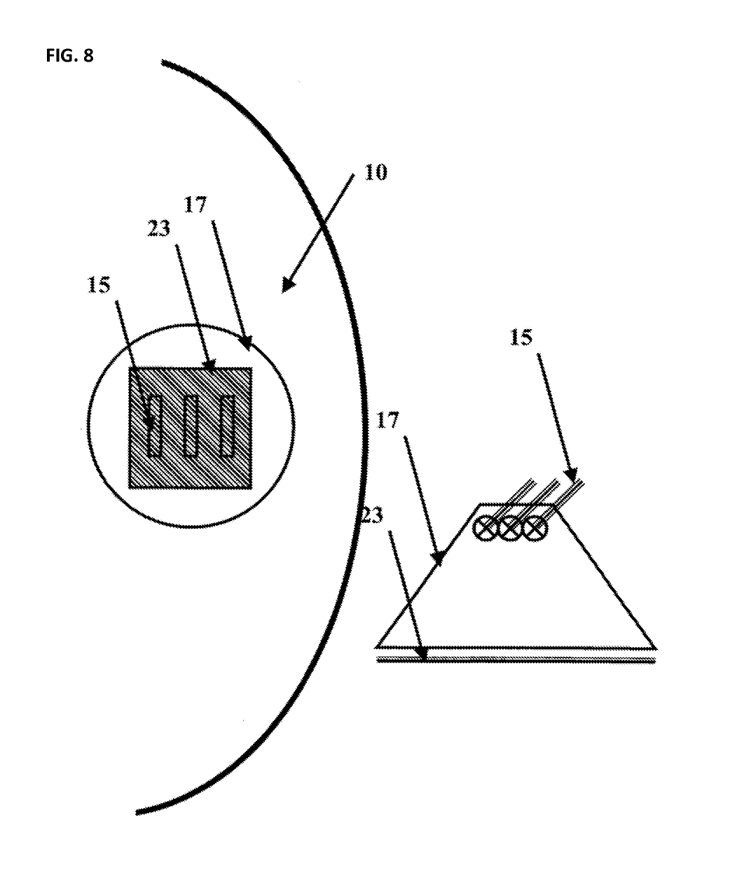

FIG. 8 shows an alternative embodiment of a skin treatment head 10. In this embodiment, a single reflector 17 encloses a plurality of lamps 15 thus allowing increased energy output from each reflector 17 in the treatment head 10. In this example, the reflector has three lamps.

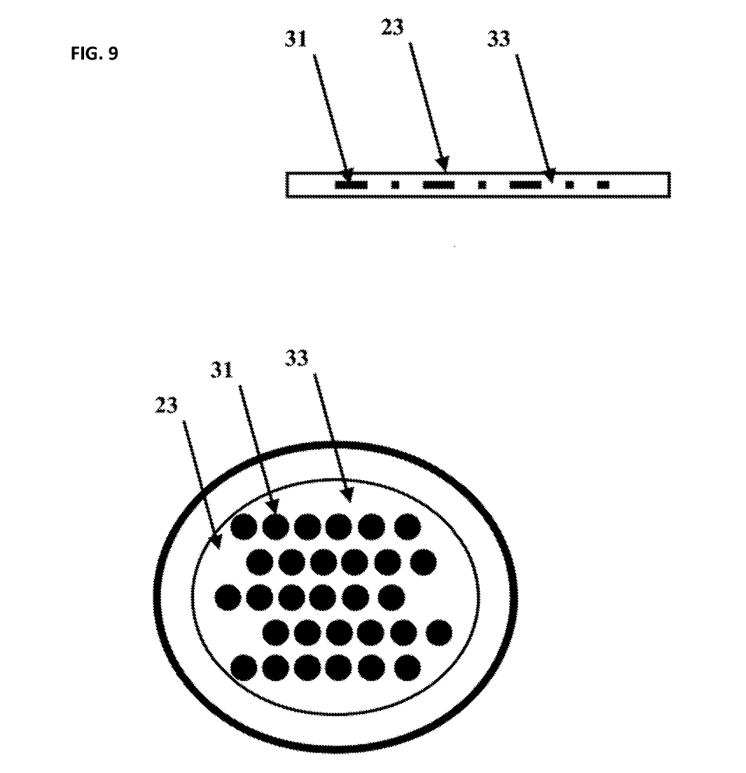

In FIG. 9 yet another embodiment is shown, wherein the high absorbing film 23 between the lamps 15 and the skin surface is made of partially transmitting material, for example, part of the film layer contain high absorbing substance 31 to absorb the light of the lamps, while other portion of the film 33 allow at least some of the optical energy through to the skin. This configuration will allow part of the light energy to be converted into heat at the skin surface and directly heat the top layers of the skin, while some of the light is allowed to propagate to deeper skin layer where a gradual absorption by skin cell heats up deeper skin tissue. In addition, some of the light that penetrates deeper skin tissue may be preferentially absorbed by skin components (for example blood vessels, or pigmentation) that may be targeted for destruction or alteration. The device in this embodiment can, therefore, serve for both skin surface treatment as well as targeting of deeper layers skin conditions.

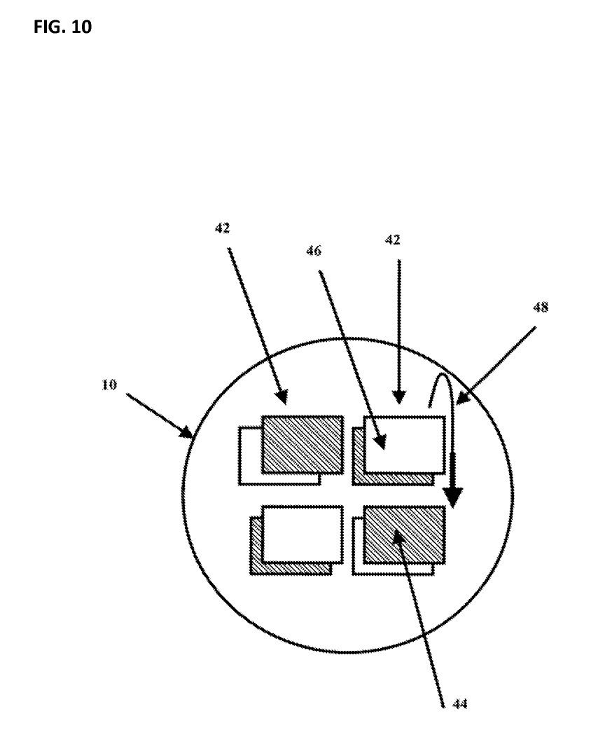

FIG. 10 shows yet another embodiment. Here the treatment head contains a plurality of treatment windows 42. Some of these windows (44) consist of a flash lamp and high absorbing substance (HAS) configuration for opto-thermal skin surface modifications (OTSSM), while some of these windows (46) contain a flash lamp and a transparent window that allows deeper skin light penetration for direct optical energy light treatments. The two types of windows (44, 46) can be mounted on a moving mechanism 48 (for example a conveyer belt type mechanism) in an alternating sequence (for example surface opto-thermal treatment window 44 followed by an optical energy treatment window 46). While the window(s) closer to the skin is/are performing the treatment, the treatment window(s) further from the skin can be charged for their turn of the treatment. Following the capacitor discharge and the treatment, the moving mechanism 48 can move the treatment windows 42 closer to the skin to the back and those in the back to the front. The treatment can then be repeated while the windows in the back are recharging.

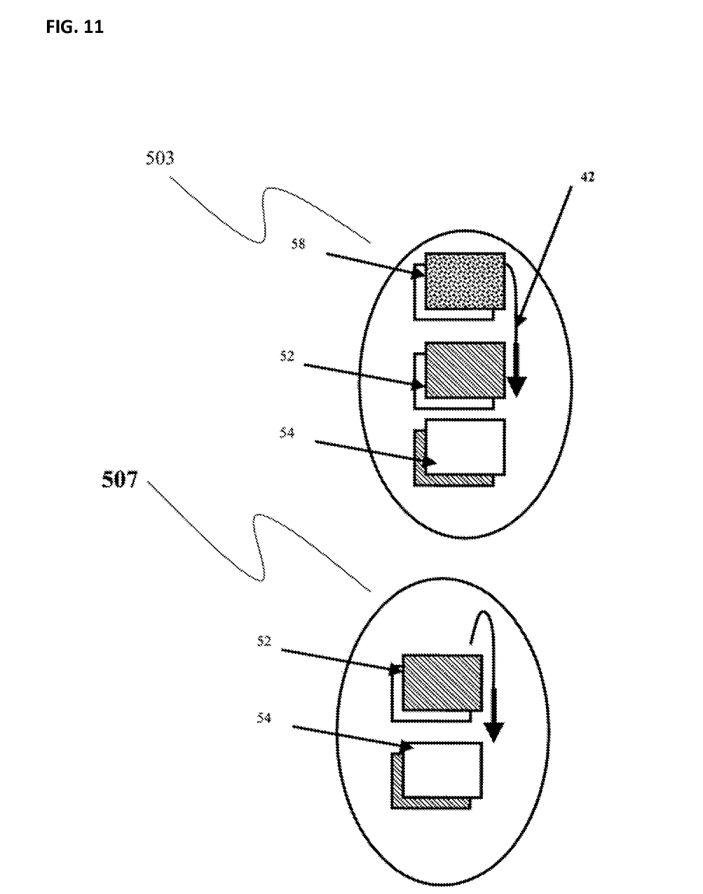

FIG. 11 shows another embodiment wherein the plurality of treatment windows 42 can be made of two (507) or three (503) windows and the treatment windows can be made of flash lamp and high absorbing substance (HAS) combination 52, a flash lamp/optical energy source 54, and an electric heater made of electric resistor for electro-thermal heating alone 58. Such a combination would allow, for example, short and rapid surface heating with the flash lamp/HAS combination, deep tissue heating with the flash lamp, and higher temperature longer heating with the electric resistor.

The main structures are the stratum corneum (a plurality of dead skin cell with a variable degree of adherence to the skin surface). The stratum corneum may vary in thickness but is generally less than 20 micrometer in thickness. Below the stratum corneum lies the epidermis which can reach as much as 150 micrometer in thickness depending on the location of the skin on the human body. Below the epidermal-dermal junction lies the dermis whose thickness is in the millimeter range and can vary considerably depending on the location on the human body.

The epidermis contains among other things, blood vessels, the nerve ending living cells, sebaceous gland, hair shafts and the roots and matrix of the body hair, sweat glands, and sweat ducts. Below the epidermis lies a layer of body fat cells.

It is generally accepted today that controlled thermal damage to the upper layer of the dermis (down to as much as 300 micrometer into the dermal layer) results, following a healing process, in production of new collagen with both improved elasticity and tightness.

A plurality of skin improvement effects by the methods comprises: depositing a controlled amount of thermal energy at the surface and allowing said energy to flow into the upper layer of the dermis, to achieve controlled damage to the collagen in the upper dermal layer. Possibly a cooling element can be activated after a predetermined time of surface heating to, remove thermal energy from the surface of the skin, protect the surface of the skin from a lengthy exposure to thermal energy, and reversing the flow of thermal energy from deeper lying layers in the dermis back to the surface; By temporarily enlarging skin surface pores and allowing cleaning of the pores and causing expulsion of unwanted debris, dirt and contaminants in the body pores, thus resulting in reduced pore size; By temporarily enlarging skin surface pores thus allowing nutrients, conditioner, and possibly drugs and medication to flow into deeper layers of the skin; By temporarily enlarging skin surface pores and allowing the expulsion of harmful sebum and bacteria thus reducing the chance for the development of acne and other sebaceous gland related ailments; By thermally damaging the surface layers of the skin followed by flaking and removal of portion of the stratum conium, and portion of the epidermis and dermis; By thermally damaging vascular or pigmented component of the skin near the skin surface (in the epidermis or upper dermis). These unwanted damaged components will then be removed by the body as waste products, eliminating disfiguring skin blemishes.

Table 1 shows approximated diffusion times for selected typical distance in water-like media such as the human or animal skin. For example the diffusion of heat to a distance of about 100 micrometer will require approximately 10 milliseconds. These diffusion times ensure that no thermal energy deposited at the surface arrives at deeper skin locations prior to these times. Knowing these approximate diffusion times the embodiments limit the extent of thermal damage to deeper skin structures by terminating the action of the energy source at the surface and possibly by introducing a skin surface cooling element subsequent to the thermal energy deposition such that the flow of thermal energy is reversed back to the surface and no thermal energy reaches below a predetermined depth.

TABLE 1
Diffusion Times

| | |
|---|---|
| Z Depth | Times |

| | | | |
|---|---|---|---|
| 1 | um | 1 | us |
| 10 | um | 100 | us |
| 100 | um | 10 | ms |
| 1 | mm | 1 | sec |

To Calculate the energy needed to increase the temperature of a given volume (Volume=Area*Depth) to a temperature DT is:

C=DE/DTÒDE=CDT

DE=CDT=c*Ro*Vol*DT

DE=cRoA*Depth*DT

Specific heat capacity water −4.187 kJ/kgK=C

Hence

DE=DT×4.2 KJ/(KG*K)

Volume=10 um×Cm2=1E−5×1E−4 m3

Volume=1E−9 m3

Density=Kg/m3

Mass=M=1E−9 Kg

=1E−6 Gram=ug

With DT=100 C

DE=4.18 (kJ/Kg)1E−9 Kg/K*100K=4.2E−7 KJ

Hence

DE=4.2 1E−7 KJ˜4 E−4J=0.4 mJ

Table 2 shows the basis for a design of a system for skin conditioning treatment based on the thermal properties of the skin. The right column shows the required energy to bring a volume of the skin with water-like thermal properties to an increase in temperatures (DT) shown in the left column. The calculations assume a skin volume of a centimeter square and depths reaching those shown in the left column.

TABLE 2

| Parameters (DT, Depth = dZ) | DE = energy needed | | Diffusion time | |
|---|---|---|---|---|
| in water | to raise and area of 1 | | (ms) to allow | |
| The area considered in | cm2 and of a depth = | | surface energy | |
| this example is generally | dZ, To Temperature DT | | to reach said | |
| about 1 cm2. | (mJ) | | depths | |
| 100 C., 10 um depth | 0.4 | J | 0.1 | ms |
| 100 C., 100 um | 4 | J | 10 | ms |
| 200 C., 10 um | 0.8 | J | 0.1 | ms |
| 200 c., 100 um | 10 | J | 10 | ms |
| 100 C., 200 um | 10 | J | 40 | ms |
| 200 C., 200 um | 20 | J | 40 | ms |
| 300 C., 100 um | 15 | J | 10 | ms |
| 300 C., 200 um | 30 | J | 40 | ms |
| 300 C., 300 um | 45 | J | 90 | ms |

Table 3 shows the particular energy delivery times of interest (ranging from about 0.1 ms to as much as about 90 ms) and corresponding to thermal diffusion depths from about 10 micrometer to about 300 micrometer well into the upper layers of the dermis. As can be seen from the tables, the energy density is in the range from about 0.1 J/cm2 to about 50 J/C cm2.

TABLE 3

| | |
|---|---|
| Thermal Diffusion distance (um) | Thermal Diffusion time |

| 10 | um | 100 us = ~0.1 ms | |
|---|---|---|---|
| 30 | um | ~1 | ms |
| 50 | um | 2.5 | ms |
| 70 | um | 5 | ms |
| 100 | um | 10 | ms |
| 200 | um | 40 | ms |

| | | |
|---|---|---|
| 300 | um | 90 ms~0.1 SEC |

Table 4 shows the ratio of thermal expansion that would result from raising the temperature of a water-like material by the additional level shown in the left column. This confirms the assertion that a sufficient volumetric expansion change will result allowing opening of the pores.

TABLE 4
Volume = 10E−9 m3 = 10E+9 um3
Thus DV/V = 700E−6 * DT
@ 100 C.
→ DV/V = 0.07
To first approximation:
DV/V ~3 DL/L
DA/A ~2 DL/L

| Delta Temp (DT C.) | DV/V (%) | DL/L % of |
|---|---|---|
| (temp increase) | Expansion ratio | Linear expansion |
| 100 | 7 | 2.3 |
| 200 | 14 | 4.7 |
| 300 | 21 | 7 |
| 400 | 28 | 9.3 |
| 20 | 1.4 | 0.5 |
| 50 | 3.5 | 1.2 |

FIG. 2a shows another possible circuit diagram to pulse the flash lamp, as described above.

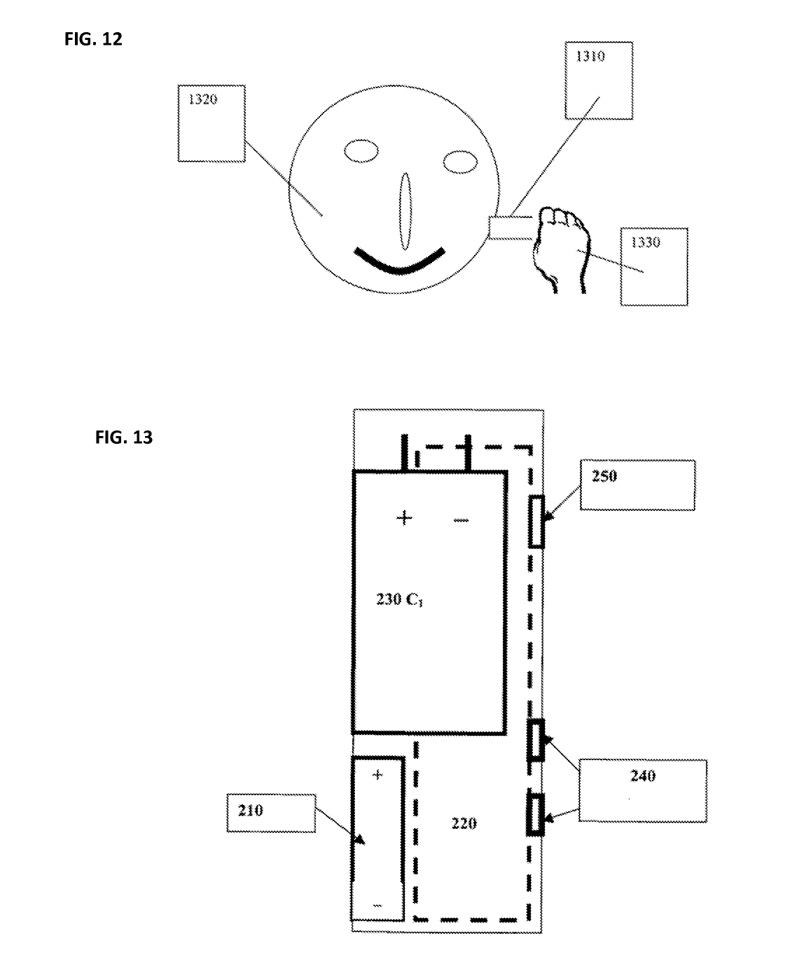

FIG. 12 shows how the device may be used to treat a blemish on the face. The device 1310 is turned on and then placed in contact with the skin 1320, when in good contact and fully charged, the fire button is pressed by the operator hand 1330 to deliver energy to the heating element which then transfer its energy to the skin. The thermal impulse to the skin acts to open pores and accelerate clearing of the blemish.

FIG. 13 shows the components driving the skin treatment device. They include a power source 210, an electronic control board 220, a capacitor 230 charged with the energy needed, a charge/fire buttons 240 and an indicator light 250 indicating that the charge cycle is completed and the unit is ready to be used.

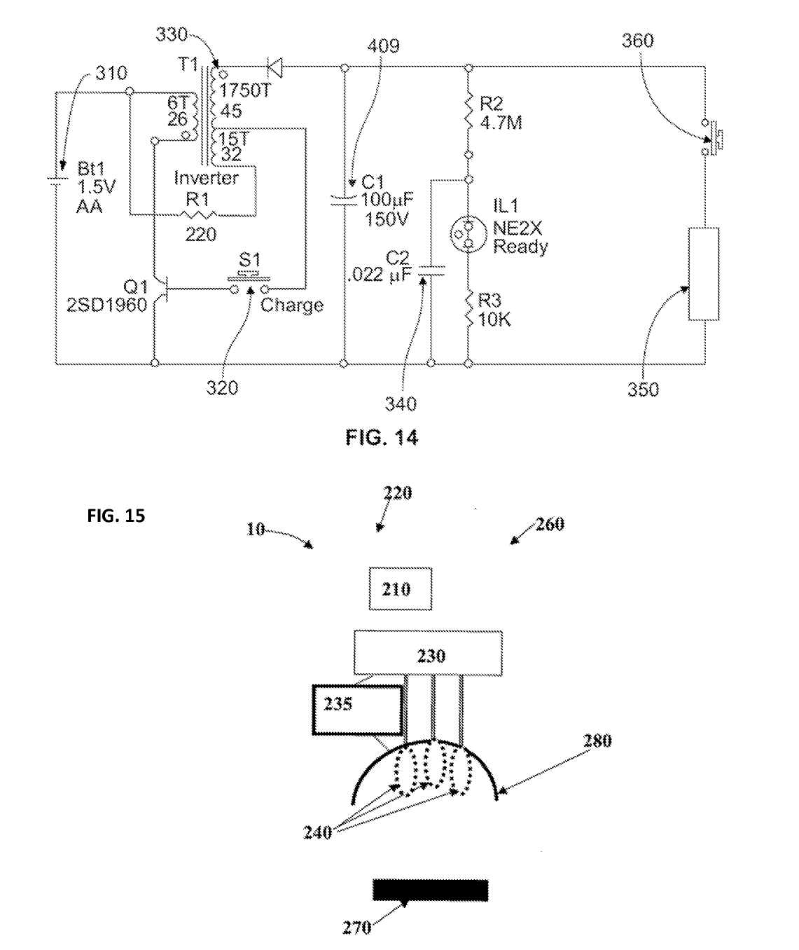

FIG. 14 is a schematic diagram for the circuit needed to drive an electric resistor energy source and transport configuration. A power source (for example a 1.5V or 6 V battery) voltage is stepped up by a voltage inverter 330 and charges a capacitor 340. A switch 320 activates this process. The capacitor 340 is discharge by a push on the fire switch 360 to heat up the electric resistor treatment head 350.

In a further embodiment, a device for treating the skin is contemplated, said device delivers a controlled amount of thermal energy to tissue and comprises: a flash lamp with an electromagnetic radiation absorbing element, a circuit to deliver a fixed amount of energy to said flash lamp, a layer of absorbing layer capable of absorbing the optical energy discharged by the flash lamp, a component capable of activating and triggering said circuit. Another embodiment contemplate a device for delivering a controlled amount of thermal energy to tissue comprises an optical absorbing element with variable transmittance properties, at least one flash lamp, a circuit to deliver a fixed amount of energy to said plurality of flash lamps, means to activate and trigger circuit.

The embodiment for a method for treating skin blemishes includes a trigger circuit to release a pre-determined amount of energy to a plurality of flash lamps, an absorbing substance capable of absorbing at least some of the light energy and converting it to thermal energy, heating a predetermined upper layer of the skin to a temperature in excess of about 50° C. The method further contemplates that the layer below the epidermal dermal junction remains below 50° C.

In another embodiment, the method contemplates keeping the layer below the mid-reticular dermis remain below 50° C. The possibility of using a cooling element is activated at a predetermined time subsequent to the heating of the skin to remove at least some of the thermal energy from the skin.

Yet another embodiment, a device for delivering a controlled amount of thermal energy to tissue comprising: an optical absorbing element with variable transmittance properties, at least one flash lamp, at least one electrical heating element, a circuit to deliver a fixed amount of energy to said plurality of flash lamps, and heating elements, means to activate and trigger circuit.

The above device also contemplates including an element for dispensing substance beneficial to skin conditioning or skin therapy is activated followed the treatment allowing delivery of said substance into the skin.

Yet another embodiment, a device for delivering a controlled amount of thermal energy to tissue comprising a resistive heating element, a circuit to deliver a fixed amount of energy to said resistive heating element, means to activate and trigger circuit. This device further includes an element that prevents electrical current from reaching the treated surface. Only the heat energy should be allowed to be transferred into the skin, but no electrical current. This can be accomplished by coating the electric heating element with electrical insulator that prevent electric current flow but allow at least some thermal energy flow.

The device for treating skin blemishes including applying a device with an element that can be quickly heated to temperature greater than 50° C. to the skin, triggering a circuit to release a fixed amount of energy to the heated element, allowing heat to conduct into the skin. The device may further comprise an electric insulation which is placed between the resistive heating element and the surface of the targeted skin but which allows thermal energy flow across it.

Further embodiments envision a therapeutic treatment device comprising: an incoherent electromagnetic energy source operable to provide a pulsed energy output from a plurality of energy sources having a spectrum of frequencies including a frequency bandwidth capable of being absorbed by an intermediate substance; a housing with an opening, said light source being disposed in said housing, and said housing being suitable for being disposed adjacent to the intermediate substance; a variable pulse-width pulse forming circuit electrically connected to said light source; a reflector mounted within said housing and proximate said light source, directing its energy towards said absorbing intermediate substance whose absorbing characteristics range from zero (completely transmitting) to infinity (completely absorbing).

The device above is contemplated to have fluence of less than about 2 J/cm2, and in a modification of the above, at less than about 1 J/cm2. Yet another embodiment contemplates the device above with an incoherent energy source which is supplemented with a laser energy directed at the general vicinity of the treatment area before, during or after the application of the pulsed energy output.

Yet another embodiment of the device above contemplates substantially depositing most of the energy of the electromagnetic source is deposited at the surface.

The device described above also envisions that substantially most of the energy of the electromagnetic energy source is deposited at the surface, resulting in expansion of skin surface opening and discontinuities to allow at least some enhancement in the transport of material across the skin to alleviate skin conditions and ailment and to improve the look and condition of the skin.

The embodiment above may also be modified to provide a device with a plurality of energy sources, such as lamps with reflectors with electromagnetic energy output and wherein at least one lamp energy is intercepted by a high absorbing film mounted proximate to the lamp opening.

Further modification of the embodiment above envisions that said energy source is a light source, a flash lamp, or a flash lamp of the type used in digital and disposable (single use) cameras.

Further embodiment envisions the embodiment of the device above wherein said energy source comprises means for providing pulses having a width in the range of between about 0.5 microseconds and 500 millisecond and an energy density of the light on the skin of more than about 0.1 J/cm2 and less than about 2 J/cm2.

Further embodiment contemplates a skin treatment device wherein said energy source comprises means for providing a pulse in the range of about 0.1 milliseconds to 2000 milliseconds, whereby skin opening may be expended to enhance transport across the skin. This device may also have an energy source comprising means for providing pulsed electromagnetic energy in the range of about 0.1 millisecond and about 1000 milliseconds, and providing laser CW light radiation before, during, or after said pulse radiation. This device may also have an energy source that comprises means for providing pulsed electromagnetic energy in the range of about 0.1 millisecond and about 1000 milliseconds, and providing laser CW light radiation before, during, or after said pulse radiation and providing lamp radiation before, during, after, and is able to heat the dermis/epidermis junction temperature to between about 45° C. and 55° C.

The device may also comprise means for providing pulsed electromagnetic energy in the range of about 0.1 millisecond and about 1000 milliseconds, and providing lamp radiation before, during, after, and is able to heat the dermis/epidermis junction temperature to between about 45° C. and 55° C.

Yet further embodiments envision the energy source which comprises means for providing pulsed electromagnetic energy in the range of about 0.1 millisecond and about 1000 milliseconds, and providing lamp radiation before, during, after, and is able to heat the dermis/epidermis junction temperature so that combined with the energy deposited in the skin by pulse EM energy source, skin conditions are alleviated including the condition of acne.

FIG. 15 shows an alternative embodiment of the handheld treatment device 200.

As shown in FIG. 15, the device has a power source 210 (a wall electric outlet, an electric transformer or a battery) that powers a circuit board 230. The circuit board 230 is activated with power switch 220 to charge a capacitor that stores enough energy to cause an electric discharge in the lamps 240. The circuit will then recharge the capacitor and be ready to fire again within a fraction of a second and up to a few seconds. In order to reduce the risk of accumulation of heat, the heating element having a high absorbing substance or other heating elements is allowed to cool down before another heating pulse is fired. In one embodiment, a temperature sensor (e.g. thermocouple) 250 may be used to monitor the temperature of the heating element and prevents a heating pulse until the temperature drops below a safe temperature (for example 35° C.). The capacitor is discharged when a fire button 260 is pushed.

In this embodiment, flash lamps 240 are used to quickly heat a thin absorbing layer 270. A circuit board 230 can fire one or multiple lamps to control the total energy delivered to the thin absorbing layer 270. A reflector 280 collects the light that is radiated away and redirects it toward the absorbing layer 270 to uniformly heat the absorbing layer 270.

In this embodiment, the high absorbing layer will be heated due to the optical energy it absorbs from the flash lamps and will then quickly transfer its energy to the skin through thermal conduction into tissue. The safety of the device is enhanced by the fact that the lamps are pulsed and they deposit a predetermined, known amount of energy into the high absorbing layer. The amount of energy transferred into the skin is, of course, always smaller than the amount of energy deposited in the optically absorbing layer.

As an example, for a 100 um thick absorbing insulator, such as a glass or plastic (capable of sustaining higher temperatures without melting) with similar thermal property, to be heated to 300° C., the energy deposited in such material layer which is initially at 30° C. is approximately 2.5 g/cc*100e−4 cm*(270 C)*0.84 J/g/C=5.7 J/cm2. If heating of the thin layer occurs within a short time compared to the thermal relaxation time, then the cooling time can be estimated from the thermal relaxation time. The relaxation time is approximately (100e−4/3.14)2/0.008=1.2 msec. For a 100 um thick copper layer heated to 300° C., the available energy to transfer to tissue that is at 30° C. is approximately 9.2 J/cm2. The relaxation time is approximately 8.65 microseconds.

Additional Embodiments are Described Below:

A therapeutic treatment device comprises: an incoherent electromagnetic energy source operable to provide a pulsed energy output from a plurality of energy sources having a spectrum of frequencies including a frequency bandwidth capable of being absorbed by an intermediate substance; a housing with an opening, said light source being disposed in said housing, and said housing being suitable for being disposed adjacent to the intermediate substance; a variable pulse-width pulse forming circuit electrically connected to said light source; a reflector mounted within said housing and proximate said light source, directing its energy towards said absorbing intermediate substance whose absorbing characteristics range from zero (completely transmitting) to infinity (completely absorbing).

In the device the incoherent energy source is supplemented with a laser energy directed at the general vicinity of the treatment area before, during or after the application of the pulsed energy output.

In the device substantially most of the energy of the electromagnetic energy source is deposited at the surface resulting in expansion of skin surface opening and discontinuities to allow at least some enhancement in the transport of material across the skin to alleviate skin conditions and ailment and to improve the look and condition of the skin.

The plurality of energy sources can be lamps with reflectors with electromagnetic energy output and wherein at least one lamp energy is intercepted by a high absorbing film mounted proximate to the lamp opening. The energy source can be a flash lamp such as of the type used in digital and disposable (single use) cameras. The energy source comprises means for providing pulses having a width in the range of between about 0.5 microseconds and 500 millisecond and an energy density of the light on the skin of more than about 0.1 J/cm2 and less than about 2 J/cm2. The energy source comprises means for providing a pulse in the range of about 0.1 milliseconds to 2000 milliseconds, whereby skin opening may be expended to enhance transport across the skin. The energy source comprises means for providing pulsed electromagnetic energy in the range of about 0.1 millisecond and about 1000 milliseconds, and providing laser CW light radiation before, during, or after said pulse radiation.

Said energy source comprises means for providing pulsed electromagnetic energy in the range of about 0.1 millisecond and about 1000 milliseconds, and providing laser CW light radiation before, during, or after said pulse radiation and providing lamp radiation before, during, after, and is able to heat the dermis/epidermis junction temperature to between about 45 degree C. and 55 degree C.

In the device said energy source comprises means for providing pulsed electromagnetic energy in the range of about 0.1 millisecond and about 1000 milliseconds, and providing lamp radiation before, during, after, and is able to heat the dermis/epidermis junction temperature to between about 45 degree C. and 55 degree C.

In the device said energy source comprises means for providing pulsed electromagnetic energy in the range of about 0.1 millisecond and about 1000 milliseconds, and providing lamp radiation before, during, after, and is able to heat the dermis/epidermis junction temperature so that combined with the energy deposited in the skin by pulse EM energy source, skin conditions are alleviated including the condition of acne.

In the device said light source comprises means for providing pulses having a width in the range of between substantially 0.05 microsecond and 1000 millisecond and an energy density of the light on the skin of less than about 10 J/cm2.

In the device said light source comprises means for providing pulses having a width in the range of between substantially 0.1 millisec and 600 millisec and an energy density of the light on the skin of less than about 6 J/cm2.

In the device said light source comprises means for providing plurality of pulses having a width in the range of between substantially 0.1 millisec and 600 millisec and an energy density of the light on the skin of more than 2.5 J/cm2.

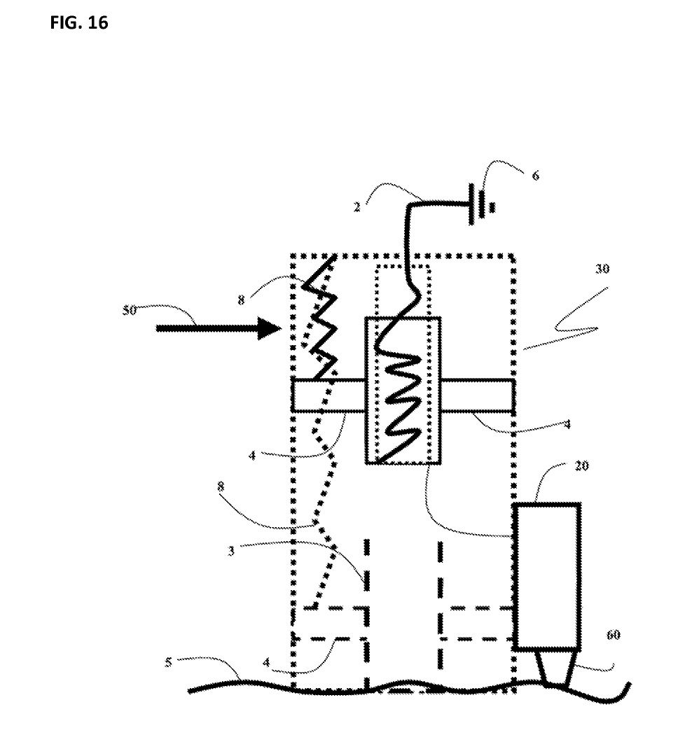

As shown in FIG. 16, a console 1 contains a heat source 2 and a heat shuttle 3 which can be brought into contact with the heat source. The heat shuttle 3 has latches 4 which allow a motion promoter 8 (for example a spring) to push it towards the skin surface or other target surface 5, and then subsequently to discharge the excess heat energy, back towards the heat source 2. The heat source 2 (for example and electrical heater) contains an energy source 6 (for example, a battery or an electrical energy source, as shown), which generates the thermal energy within the heat source. Said thermal energy is subsequently delivered to the skin by means of the heat shuttle 3 or by forming contact with the target allowing thermal energy to diffuse directly into the skin.

FIG. 16 also shows the position of the heat shuttle 3 with respect to the target material surface 5 and the heat source 2 (for example, a winded wire resistor or some other type of thermal energy generating electrical resistor), when in contact with the skin. Note the extended form of the motion promoters 8.

As is also shown in FIG. 16, the device can also be envisioned to work in combination with a dispenser of a drug or nutrient or any other substance that one desires to deliver into the surface and in particular into the skin. A container 20 carrying the desired substances can be attached to the device 30 and as the device is moved as shown by the direction of the arrow 50. The container 20 dispenses its substance through a dispenser 60 which can be brought into contact with the target surface and in particular with the target skin. If the dispenser assembly 20 and 60 precedes the action of the HS device 30 as when the motion is in the direction of the arrow 50, then the HS device 30 acts on the material to drive it into the target surface or skin. However, if the dispenser assembly 20 and 60 follows the action of the HS device 30 as when the motion is in the direction of the arrow 40, then the HS device 30 acts on the target material or skin to modify said target surface or skin and enhance the material that is delivered subsequent to the HS device 30 action.

In another embodiment, laser source (preferably a diode with continuous wave (CW) emission power of about 0.5 W to about 10 W and preferably with a CW emission power of about 1 W to about 2 W) is focused to a line (e.g. ˜1 cm long) with a cylindrical lens.

The device comprises: a trigger that releases a hook, a hook that holds a mirror that is spring loaded, a spring that forces the mirror to move thus moving the line, a scanned line that makes a rectangle scan of about 1 cm×1 cm in area, a small electric motor then reloads the spring/mirror to its original position and the hook latches back on.

The trigger also releases two other safety shutters:

1. One is connected to the electrical motor and is designed to flip open/shut a bit slower than the time it take the mirror to do its scan.

2. The second is mechanical and can either be designed to close automatically (e.g., a spring loaded one and its hook is designed to release a spring that closes it e.g. 10 ms after the scan begins.

Or it can be designed to remain open as long as the finger is on the trigger.

The light scans an area that is larger than the opening of the device. The opening of the device is design to allow only the approximately linear and constant velocity of the scanned light through, i.e., the acceleration/deceleration portions are cut out of the opening and do not make it out of the device.

The light scans the surface of a HAS film, which may be called a “bullet”. The bullet comes out of a magazine loaded with e.g. about 30 bullets. 30 Bullets should be enough to cover an entire face. The bullets in the magazine are spring loaded and come out with each device trigger action. Each trigger action also removes the old bullet (e.g. the new bullet pushes the old out) into a disposable collector. Each bullet may be soaked with a lotion for anti-aging or wrinkle treatment, Oil of Oley, acne ointment, nutrients vitamins or any other substance that one may wish to deliver trans-dermally.

Alternatively, a reservoir of said desired fluids or creams to be delivered trans-dermally into the skin or any other target surface may dispense the desired material either before, during or after the light scanning action.

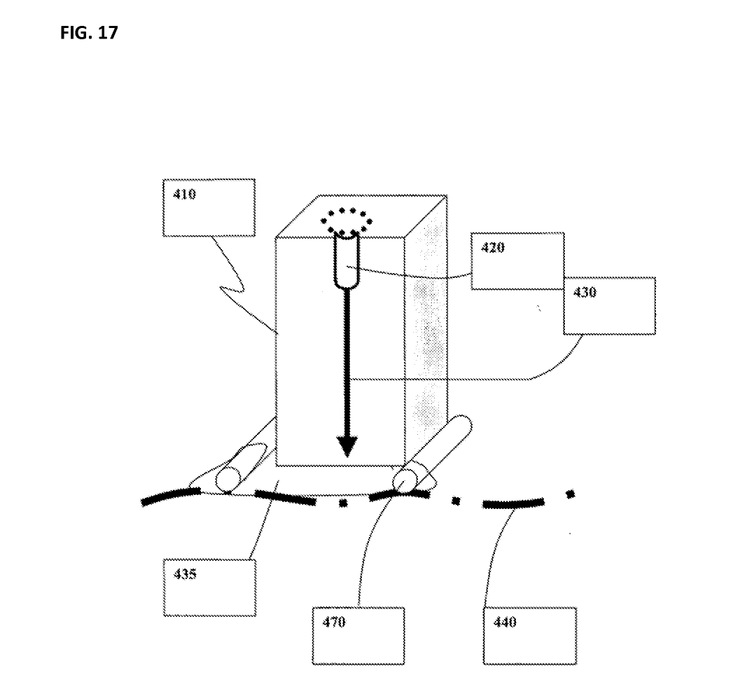

FIG. 17 illustrates yet another embodiment. In this embodiment, the energy source 420 contained within the encasing 410 is a broadband emitter of energy. In yet another embodiment, the energy source is a source of electromagnetic radiation and preferably a broadband electromagnetic radiation with a spectral range from about 350 nm to about 2000 nm and preferably from about 400 nm to about 1100 nm.

In a modification to this embodiment, the energy source 420 is a flash lamp, preferably a flash lamp with approximately the same characteristics as those of most disposable one-time use camera on the US market. In this embodiment, such energy sources are light source with small flash lamp capable of illuminating a field of up to 20 feet and are powered by a 1.5-volt battery or two 1.5 volt batteries and at least one capacitor and the electronic circuitry to discharge and recharge it.

In an embodiment, a high absorbing substance (HAS) film 435 or a partially transmitting HAS film 435, which is mounted on rollers 470, is used to convert at least some of the flash lamp's energy into thermal energy. The film is in contact with the targeted surface or skin and thus is capable of transferring said converted optical energy from the flesh lamp to the film and to the target surface or skin so that a beneficial change to the skin condition or the target surface does occur.

In yet another embodiment said targeted HAS film is made of disposable material either on roller or on removable disposable caps so that it is replaced from energy discharge to the next or from use to use or from time to time. In another embodiment, the flash energy source or the entire assembly is a single use or made to be used only for a few firing of the energy source and then being replaced from time to time.

Here, the light from the energy source 420 (preferably a laser) impinges on the a film (407) saturated with a substance of high absorbance in at least one spectral band of energy radiation 330 coming out of the energy source 420. The energy beam 420 then interacts with the film and its energy is converted into thermal energy that subsequently propagates into the targeted surface or skin 440. A set of rollers 470 dispenses the disposable containing high absorbing substance film and collects it on the other side.

Alternatively, the film 440 can be made of a pattern of absorber regions and transmitting regions wherein the absorbers can be made, for example, in a pattern, a pattern of absorbing dot matrix or absorbing lines and the rest of the film is made of transmitting material.

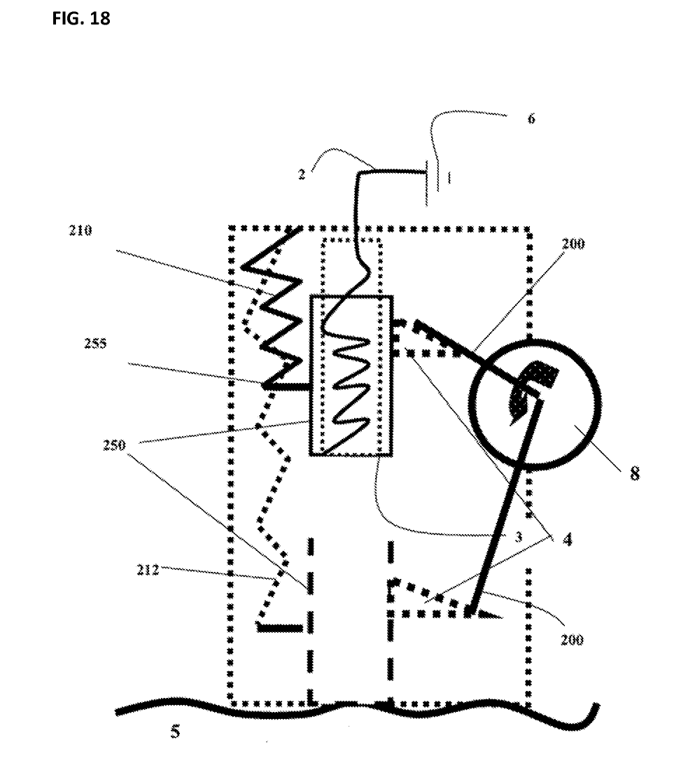

FIG. 18 describes another embodiment. Here the device 30 is modified so that the motion promoter element 8 of the heat shuttle is a motor, preferably an electrical motor. As the motor turns, it pushes with its bar 200 on the latches 4 which in this case is in the shape of a wedge as shown. As the motor spins, the latch 4 along with the heat shuttle is pushed downward. The latches 4 and the bar are designed to be in contact so that the motor pushes all the way to the skin or target surface 5. When contact is made, the bar 200 continues to push again the latches down so that the heat shuttle is forced into a good contact with the skin. The bar 200 at that time is just about clearing its contact with the latches 4. The latches 4 are made of somewhat flexible material (e.g. like a hard rubber rod) and as the motor 8 continues to push the bar 200 again the rubber latches 4, the bar slips off the latches wedge and the latches are no longer pushed by the motor 8 and its bar 200. The heat shuttle is spring loaded with a spring 210 as shown, and is thus pulled back all the way up and back into contact with the heating element 2. Position 212 shows the spring in its extended position.

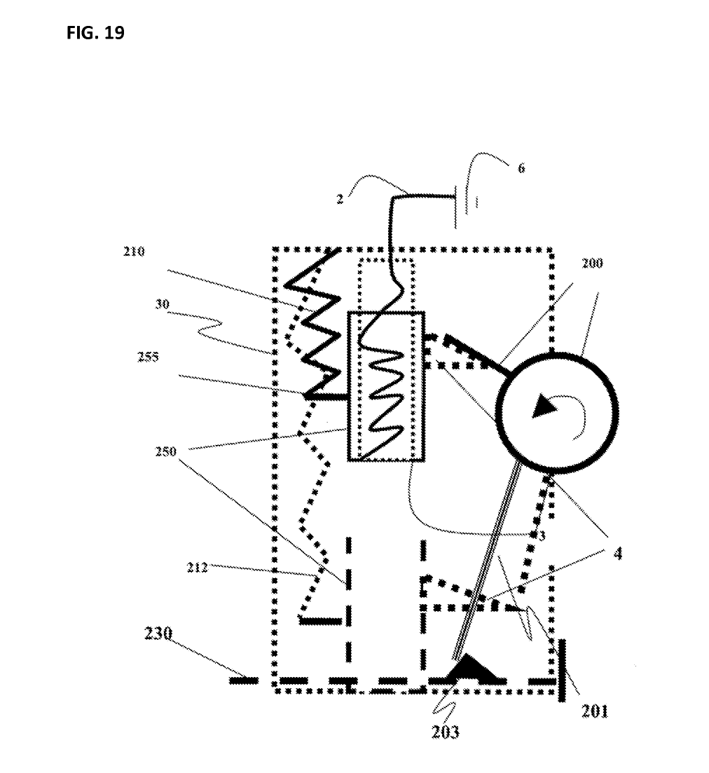

Alternatively, in another embodiment shown in FIG. 19, the motor also actuates in a simultaneous motion a second bar 201 that pushes against another wedge 203 that is connected to a shutter 230 causing it to open as the heat shuttle descend. With the same mechanism utilizing the motor 8 rotational motion and the wedge 203, at some point, the wedge 203 is released and spring 240 pushes the shutter back to cover the target the surface. Wedge 203 can also have other shapes such as a bar or a projection. The complete clearing of the device 30 opening by the shutter is designed to happen just before the HS is about to make contact with the target surface or skin. As the shutter is pushed back by the spring 240 it may be utilized to push out the bottom portion of the HS 250 which is thus made to be a disposable part utilized only once in each contact. (i.e. a disposable “bullet” in the description above).

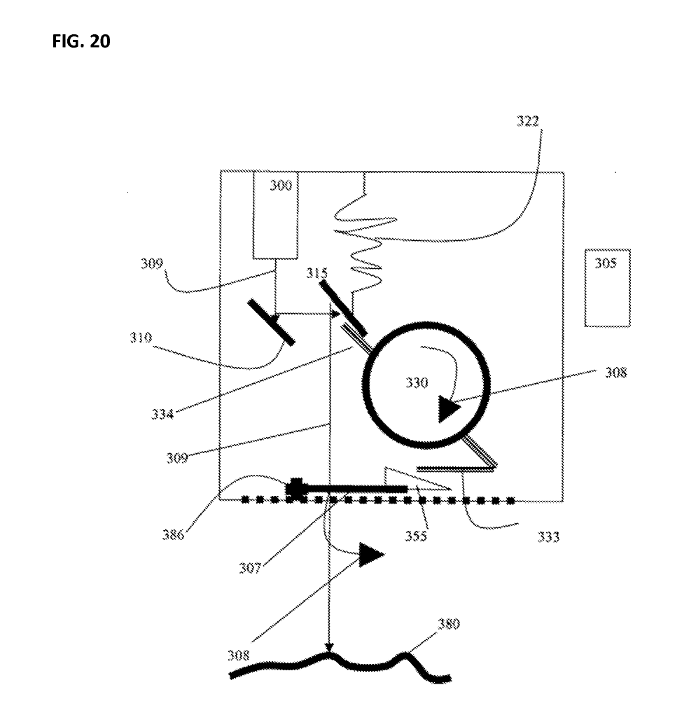

As shown in FIG. 20, another embodiment utilizes the dual push mechanism described by FIG. 20 to generate a mechanical scan synchronized with the action of a shutter to ensure safety and automated shut off.

A continuous wave laser 300 is activated when an on/off trigger 305 is pushed. The on/off trigger also opens a master shutter 307. The on/off switch also trigger the rotation of a motor 330. Two bars 333 and 334 which are attached to the motor move the mirror 315 and the hedge attached to the shutter 307. The spring 322 is compressed during the motorized wheel motion to move the mirror and once the bar 334 releases the mirror 315, the spring 322 pushes it back to its original position. The beam from the laser 300 bounces off the mirror 310 to the swinging/scanning mirror 315 and then out through the opening when the shutter 307 is swung open.

In this embodiment an exemplary operation of the device shown in FIG. 20 utilizes a bar 334, bar 334 is pushed against the scanning mirror 315 which is then moved (in this case upward) at the desired rate. When the bar 334 slips off the mirror (the mirror edge can be shaped as a wedge to facilitate such slippage) the mirror 315 is pushed back rapidly by a spring 322 that returns it to its original position. The rotational motion of the motor 330 provides a uniform scan rate for the mirror.

Simultaneously to this motion, the other bar 333, which is attached to the motor 330, is pushed against the wedge 355 to cause a second shutter 307 to be open (in the direction of the arrow 308) at a uniform rate. As the shutter 307 swings open it allows the scanning laser beam 309 to be moved synchronously with the motion of the scanning mirror to allow the beam through the shutter 307 and into interaction with the target surface or skin 380. When the bar 333 slips off the ledge 355 the shutter 307 is rapidly pushed back by spring loading component 386 forcing the shutter 307 to its close shut, thus preventing the beam from reaching the target surface or skin 380.

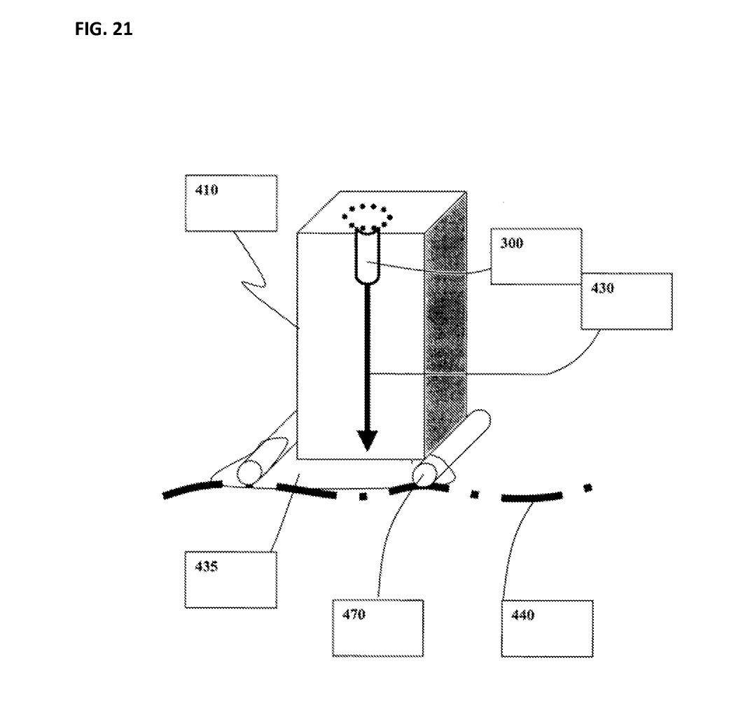

As shown in FIG. 21, yet another embodiment pertains to opto-thermal interaction with a target surface or a skin as shown in FIG. 21. Here, the light from the energy source 300 (preferably a laser) impinges on a film 435 saturated with a substance of high absorbance in at least one spectral band of energy radiation 430 coming out of the energy source 300. The energy beam 430 then interacts with the film and its energy is converted into thermal energy that subsequently propagates into the targeted surface or skin 440. A set of rollers 470 dispenses a disposable film containing high absorbing substance film and collects it on the other side. Alternatively, the film 435 can be made of a pattern of absorber regions and transmitting regions wherein the absorbers can be made, for example, in a pattern, a pattern of absorbing dot matrix or absorbing lines and the rest of the film is made of transmitting material.

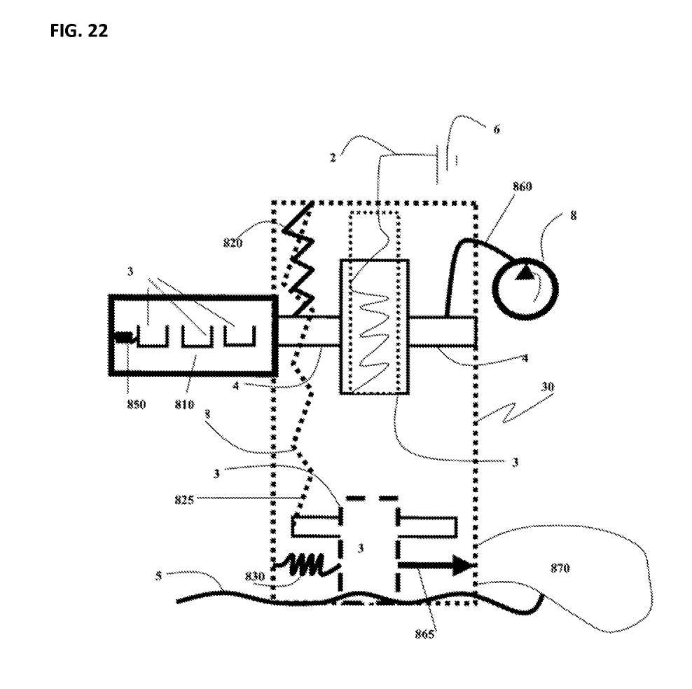

FIG. 22 illustrates a device 30 for electro thermal surface treatment (including skin conditions such as acne) wherein the heat shuttle 3 is now a disposable element that is stored in a magazine (or clip) 810 full of additional disposable heat shuttles 3 (like “bullets” stored in a clip).

A spring 850 propels the “bullets” heat shuttles 3 towards the heating element energy source 2 where the bullets 3 are secured and kept in contact with the heat source through the force provided by a spring 820. A motion propeller 8 which can be an electric motor 8 pushes on the latch 4, and move the heat shuttle away from the heat source and into contact with the target surface or skin 5.

Once in contact with the target surface or skin 5, the motion promoter (e.g. an electric motor) arm 860 slips off the heat shuttle handle bar 4 and no longer forces a pressure of the heat shuttle 3 on the target surface or skin 5. At that time, a removing mechanism consisting of a spring 830 is released and pushes the used heat shuttle 3 away from the skin as shown by the arrow 865 and into a disposed heat shuttle collecting pouch 870.

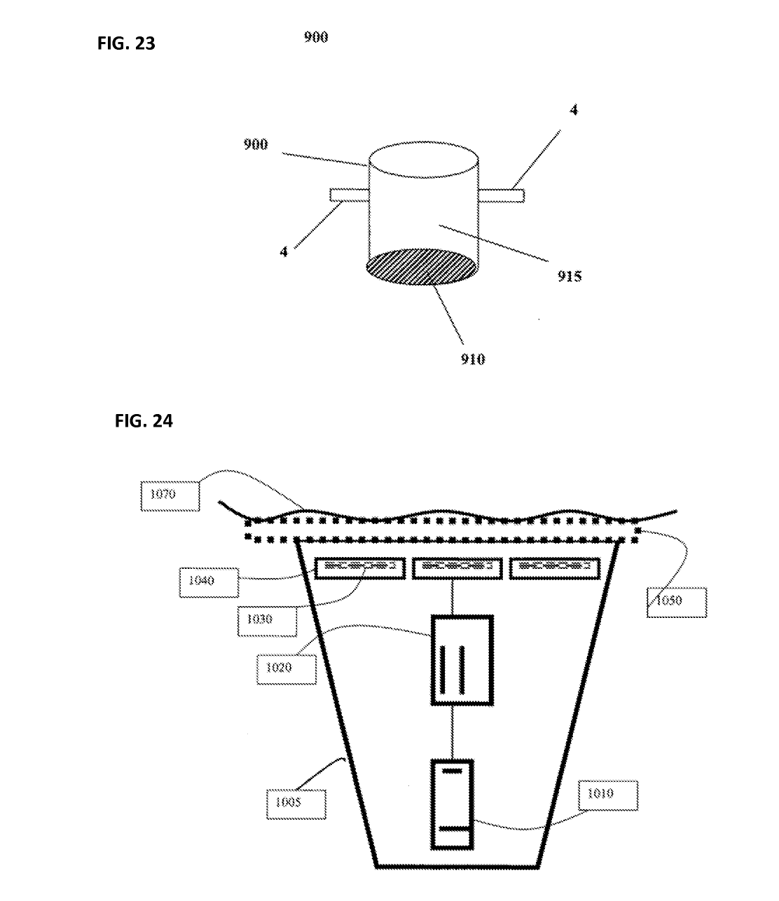

FIG. 23 illustrates an exemplary composition and construction of a disposable heat shuttle 900 as used in the embodiment of FIG. 22. The body of the heat shuttle 900 is made to fit around the heat source. The body 915 of the shuttle can be made for example from an insulating material, for example, a plastic, glass, or Teflon that are capable of withstanding high temperature (for example up to about 400 to 500 degree C.) without deformation or chemical changes to them. The body 915 can also be made of metal (for example, copper, or aluminum) to allow heating of the body 915 itself and not just the active element 910 at the bottom. There is at least one bar or latch 4, which is used to push the heat shuttle 900 against the heat source. At the bottom of the heat shuttle 900 there is an active element 910 for thermal energy storage and capable of contacting both the heat source for the purpose of uploading thermal energy and, subsequently, for contacting the target surface or skin, for the purpose of conducting its thermal energy to the target surface or skin and unloading its thermal energy to the target surface or skin. The active element 910 can be any material capable of being heated by a hot body such as an electrical heater or an soldering iron. The active element 910, however, must be capable of easily conducting its thermal energy into the target skin. Therefore the active element 910 is preferably made of metal such as copper, or aluminum.

FIG. 24 shows yet another embodiment. Here, a housing 1005 contains the entire apparatus. An energy source 1010, (for example, can be a 1.5V AA battery or two of them) charges a capacitor 1020. The capacitor discharge allows a flash lamp (or other component capable of generating electromagnetic energy) 1030 to emit electromagnetic energy of known amount in known time duration (these can be easily calculated by a person skilled in the art). The generator of electromagnetic energy or flash lamp is positioned inside a lamp housing 1040. A thermo-optical converter 1050 then absorbs the electromagnetic energy and converts it into heat. The thermo-optical converter 1050 can be brought close to or in contact with the skin 1070 and transmits the thermal energy to the skin. In an alternative embodiment, the thermo-optical converter 1050 is composed of some portion that are fully transmitting of the electromagnetic energy, some portion are partially transmitting and partially absorbing the electromagnetic energy, and some portions of the thermo-optical converter are fully absorbing of said electromagnetic energy or flash lamp energy. The amounts of energy that are fully absorbed, fully transmitted, and partially transmitted and their location on the thermo-optical converter 1050 surface, can be varied according to the desired effect and how much energy is desired at each surface location versus how much energy the user wish to allow to penetrate the surface and heat the surface below.

In another embodiment, multiple flash lamps or electromagnetic energy generators 1030 (and their related energy sources 1010, and capacitors 1020) are packed into a single housing 1005 to allow the user larger area coverage or to increase total energy delivery into a desired treatment area. In another embodiment, said multiple flash lamps or electromagnetic energy generators 1030 are willfully triggered in a desired sequence and multiple times to create a repeated illumination of the same electro thermal converter surface area or a pattern of sequential illumination of different regions within the opto-thermal converter area or a combination of the two.

The present embodiments propose and utilize the concept of thermal energy application to modify the skin or target surface condition to allow modification of the surface for treatment of hair follicles conditions and sebaceous gland conditions. The idea is based on the relative expansion and forced separation of adjacent points on an elastic surface. Just like an expanding balloon, where the relative distance with the expansion of the universe, so do the boundaries of the pores and indeed every point on the expanding skin. Each point on the surface of the balloon is separating and increasing its distance from its neighbor. If one draws a hair follicle opening on such a surface it is clear that said hair follicles opening boundaries are increasing in size with said expansion. Since different material increase at different spatial rate with increase temperature (and increase thermal energy) the result is a disruption in the bond of a plug in the pore opening of the hair follicles and the pore walls occurs. Such result allows dislodging of the plug and enhanced drainage of the unwanted material from inside the surface of the target material or the skin to the outside.

In another embodiment, one may add a substance with high coefficient of thermal expansion to the opening of the pore. One may also try to force such a substance of high thermal expansion coefficient into the target surface opening or skin pores. Such a substance may increase and enhance the relative displacement of the pore opening walls with respect to the plugging material and debris that cause the plugging.

The embodiments are based, at least in part, on the discovery that energy can modify skin structure in a reversible way so as to mitigate sebaceous gland caused conditions as well as cure sebaceous gland disorders, e.g., eliminate, inhibit, or prevent occurrence or reoccurrence of the skin disorder. An example of such a sebaceous gland disorder is acne.

Since many undesirable skin conditions result from the blockage of the skin pores, a method for changing the skin pore size and ability to transport fluid was developed using thermal energy. Thermal energy causes material to expand. The exact extent, manner, and amount of expansion are dependent on the parameters of the energy application process. In addition, the extent of the collateral effect (e.g. collateral damage or nature of changes to the skin tissue or target material) is also dependent on parameters of energy application.

In its most general form, continuous application of large amount of energy will cause expansion of the skin or target material but said applied energy will also diffuse into the tissue and may cause unwanted damage to the dermis or deeper lying structure of the target material.

In one embodiment, thermal energy is applied substantially to the surface of the material or skin in quanta. It can also be brought about via the use energy quanta loaded onto a shuttle that carries that energy from a heat source to the target material or skin. If said energy quanta is unloaded in a rapid manner, (as would be the case for example, when a heated metal body contact the surface of the skin) its excess energy would rapidly flow into the surface of the material and substantially remain there for a duration which is dependent on the thermal conductive nature of the skin or target material. This action creates a pulsed heating of the skin and has the additional advantage of predetermining the total amount of energy delivered to the skin.

With knowledge of the thermal conductivity of the skin, one can calculate what is the amount of energy that is launched into a predetermined volume and the time-dependent characteristics of such a heating process. In one embodiment, heating of the upper volume of the skin (for example, from about 5 um depth and down to about 300 um from the surface of the skin,) to a temperature of from about 30 degree centigrade and up to about 400 degrees centigrade for duration of up to about 100 ms. Such a heating range will cause sufficient thermal expansion to allow material to enhanced material flow in and out of the skin pores. The process can then be repeated by removing the energy transporter form the skin and either reloading it with energy to be delivered to the skin or target material or loading a new transporting element with energy and repeating the process.

Depending on the desired effect, the process can be repeated either in such a way that allow dissipation of the energy that was deposited in the skin by the preceding energy transporter, (i.e. so that the temperature of the skin return to its normal level and all excess energy has been dissipated) or in such a way as to build up in cumulative energy deposition so that beyond the spikes in energy build up there is also average temperature increase in of the upper layers of the skin.

Such cumulative energy built up the associated temperature increase can be useful in, for example, enhancing circulation, stimulating collagen build up, stimulating healing, enhancing activity and penetrating of drugs and substance that have beneficial effects if delivered into the skin, enhancing removal of substances that has bad influences or negative effect on the health or well-being of the skin. Such material and sebum removal can be aided by a preceding, simultaneous or following actions of vacuum pumps and suction devices.

Such deposition can be aided by a preceding, simultaneous or subsequent substance delivery action such as ultrasound, electrophoresis or any other devices or methods that allow substance to be driven or pushed into the skin. The energy quanta delivery process has the additional advantage of predetermining the collateral effects and collateral damage of the process or the device. This is the case because if no excess energy is loaded into said energy shuttle no excess damage can occur.

The embodiments disclosed herein are based, at least in part, on the thermal energy action being used to treat sebaceous gland disorders, e.g., eliminate, remove, or prevent occurrence or reoccurrence of the sebaceous gland disorder. Examples of such sebaceous gland disorders include sebaceous gland hyperplasia, acne vulgaris and acne rosacea. An example of such a sebaceous gland disorder is acne.

The methods for modifying the opening to the infundibulum comprise applying thermal energy to the opening to the infundibulum. A sufficient amount of the energy is deposited at the surface of the skin to causes an expansion of the region of the infundibulum, thereby modifying the opening to the infundibulum. In one embodiment, the opening to the infundibulum is altered such that pore pluggage will not occur, e.g., the infundibulum shape is modified temporarily or permanently such that excess sebum, oils, dirt and bacteria will not cause pore pluggage to occur, resulting in a blackhead (comedon) or white head (milium). In a embodiment, the opening to the infundibulum is opened.

Sebaceous glands are components of the pilosebaceous unit. They are located throughout the body, especially on the face and upper trunk, and produce sebum, a lipid-rich secretion that coats the hair and the epidermal surface. Sebaceous glands are involved in the pathogenesis of several diseases, the most frequent one being acne vulgaris. Acne is a disease characterized by the occlusion of follicles by plugs made out of abnormally shed keratinocytes of the infundibulum (upper portion of the hair follicle) in the setting of excess sebum production by hyperactive sebaceous glands. Various treatment modalities for acne exist that aim in modifying the rate of sebum secretion by the sebaceous glands (e.g., retinoids), inhibiting the bacterial overgrowth in the follicular duct (antibiotics), or decreasing the inflammation of acne lesions (anti-inflammatory agents). Most of these agents are not curative of acne and simply control the disease by affecting one of the aforementioned pathogenic factors. Oral retinoids are a notable exception: they are potent drugs that can achieve a significant cure rate for acne, but their side effect profile often limits their use. The treatment can permanently or temporarily (and reversibly) alter the pilosebaceous unit, rendering it no longer susceptible to pore pluggage but without the side effects associated with oral retinoids.

The term “sebaceous gland disorders” is intended to include those sebaceous gland disorders which can be treated by the delivery of thermal energy.

Thermal energy quanta can interact with the site of pore pluggage, inflammation, bacteria, viruses, etc. and promote, for example. Examples of sebaceous gland disorders, which can be treated by the methods, include sebaceous gland hyperplasia, acne vulgaris and acne rosacea. Of particular importance is treatment of acne by the method disclosed herein.

The term “pluggage” is intended to obstruction of the pores by the buildup of sebum, dirt, bacteria, mites, oils, and/or cosmetics in the pore, e.g., about the infundibulum.

The term “acne” is recognized but those skilled in the art and is intended to include acne vulgaris and acne rosacea. Acne vulgaris the most common skin disease seen in dermatologic practice which affects approximately 17 million people in the United States. Its precise cause is unknown, although abnormal keratin production with obstruction of the follicular opening, increased production of sebum (lipids secreted by the androgen-sensitive sebaceous glands), proliferation of Propionibacterium acnes (anaerobic follicular diphtheroids), follicular rupture and follicular mites (demodex) are commonly associated with acne.

Skin conditions such as acne are believed to be caused or exacerbated by excessive sebum flow produced by sebaceous glands most of which are adjacent to and discharge sebum into, hair follicles. Sebum is composed of keratin, fat, wax and cellular debris. Sebum forms a moist, oily, acidic film that is mildly antibacterial and antifungal and may to some extent protect the skin against drying. It is known that the bacteria which contribute to acne, Propionibacterium acnes or (P-acnes), grows in sebum. Significant sebum flow in humans begins at puberty. This is when acne problems generally arise.

The term “thermal interactions” (therapeutic, conditioning, or simulative) is recognized by those skilled in the art and is intended to include interactions, which are due to conversion of energy into various form of thermal energy or heat. For example, incident electromagnetic energy or light impinging upon a substance capable of absorbing such energy causes the absorbing substance to be energized and the material becomes heated. Further transmission of the energy to the target material via conduction, convection, or radioactive transfer result in the heating of the target area, preferably selectively with a significant temperature increase of such that unwanted material, e.g., tissues, oils, bacteria, viruses, dirt, etc. are removed. Preferably, the target heating is such that the surrounding tissue remains unaffected. The photothermally or thermally targeted material can also form biologically reactive products that further inhibits skin disorder or modify and condition the target material. Such thermal activation processes can involve oxidation of, for example, cell walls, extra-cellular matrix components, nuclei, etc. As a result of thermal action, the infundibulum can be temporarily or permanently reshaped. Additionally, the process can cause cell death in the sebaceous gland, thereby decreasing production of sebum.

Thermal alteration of the follicle infundibulum requires the deposition of sufficient energy to cause local heating to temperatures capable to bring about sufficient volumetric changes in the tissue. In general, these temperatures range from about 30 degree C. to about 500 degree C. for a range of expansion of the pore opening and preferably from about 50 degree C. to about 350 degree C.

The time duration of the thermal energy deposition which is sufficient to cause thermally induced changes in the blocked region of the follicular opening, can be determined by considering the basic principles of thermal diffusion. If the thermal energy is delivered within the thermal relaxation time for the target structure, heat flow from the target volume is limited during the thermal delivery time. The thermal delivery time is therefore about equal to or less than the thermal relaxation time of the given target, which measured in seconds is approximately equal to the square of the target's shortest dimension measured in millimeters.

In most skin disorder treatments that involve minimizing the effect to the non-vascular part of the skin (layers without blood vessels or capillary) the interaction should be confined to the epidermis. If the epidermal thickness to be on the order of about 100 micrometer, the thermal diffusion time is on the order of about 10 millisecond. The thermal energy delivery phase to the skin should thus be confined to less than about 10 millisecond.

As another example, the infundibulum portion of most sebaceous follicles on the face is approximately 0.3 mm in diameter and the relevant depth is also on the range of about 0.1 mm to about 0.4 mm and preferably about 0.2. mm

This corresponds approximately to a thermal relaxation time of from about 0.01 seconds to about 0.1 seconds (100 ms). In practice, the thermal diffusion into the relevant tissue depth in time duration is sufficient to achieve thermo-mechanical expansion of the skin within the heated volume. The collagen shrinkage is not contemplated as mean for achieving changes in the follicular opening to the skin as in the Anderson patent.

Although thermal confinement can achieved with laser pulse energy, for example pulses shorter than the target's thermal relaxation time, very short pulses cause unwanted mechanical injury, which can rupture the follicles. The fatty acids, sebum, and bacteria present in sebaceous follicles are extremely irritating if not contained by the follicle. In acne vulgaris, rupture of the follicle is an event, which stimulates inflammation to form a “pimple”, including accumulation of pus to form a “whitehead”. It is therefore desired to avoid rupture of the follicle or sebaceous gland.

The method for avoiding such mechanical injury is by allowing the surface of the skin to expand like a membrane or a balloon surface. A weak location at or near the skin surface in the infected area or pimple is the connection of the plug material to the wall of the follicle which. Thus, when the targeted surface is forced to expand, the expansion allows separation of the plug boundaries from the walls of the follicle opening and at least some opening between the follicle walls and the plugging material. This, in turn allows drainage of the infected interior. The expansion of the follicle opening may allow excess sebum, oils, dirt and bacteria to be expelled so that pore pluggage will not occur, avoiding such conditions as black heads (comedon) or white heads (milium).

Alternatively, a material capable of enhanced absorption of energy may be selectively deposited only at the follicular opening and be caused, after being activated through contact with hot (thermal energy loaded) material, to expand and thermo mechanically push the walls of the opening of the follicle allowing them to expand. Such thermal energy activated material that expand as a result of contact with the hot item can be, for example, animal fat or any other material that has larger volume expansion coefficient than the skin (or any target surface) itself.

The calculation for a simple model of target material water-based volume expansion and temperature increase is illustrated below.

1) Energy needed to increase the temperature of a given volume (Volume=Area* Depth) to a temperature DT is:

C=DE/DTÒDE=CDT

DE=CDT=c*Ro*Volume*DT

DE=cRoA*Depth*DT

Specific heat capacity water −4.187 kJ/kgK=C

Hence

DE=DT×4.2 KJ/(KG*K)

Volume=10 um×Cm2=1E−5×1E−4 m³

Volume=1E−9 m³

Density=Kg/m³

Mass=M=1E−9 Kg

=1E−6 Gram=ug

With DT=100 C

DE=4.18 (kJ/Kg)1E−9 Kg/K*100K=4.2E−7 KJ

Hence

DE=4.2 1E−7 KJ˜4 E−4J=0.4 mJ

Para (DT, Depth=dZ) In water

The area considered in this example is generally about 1 cm2.

DE=the energy needed to raise and area of 1 cm2 and of a depth=dZ, To Temperature DT (mJ).

Finally Additional Embodiments are Described Below:

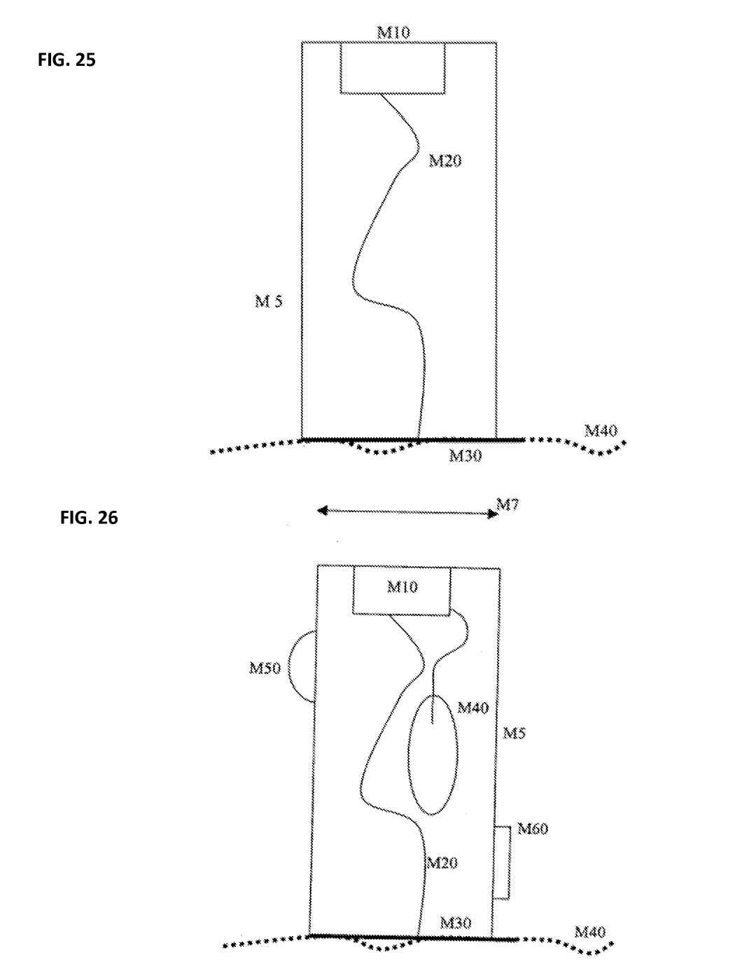

FIG. 25 shows a method for treating skin conditions including acne by means of generating heat at the surface of the skin so that skin conditions are alleviated or improved.

In one embodiment an energy source M10 is caused to willfully generate energy that is conducted by intermediate media M20 to a treating head M30 which is in contact with the skin. Such energy source can be made, for example from an electrical energy source such as a battery or an electric power supply or an electric plug. A conducting intermediate media can be made for example from electric wires and the treating head can be made, for example from an electric resistor capable of generating heat which is then conducted to the skin. The enclosure M5 may hold the entire device or the power source may be external to the container M5. If the device is designed to be handheld the enclosure M5 should be of a size that is easily held by the palm of the hand of even a petit operator. Thus the lateral dimension M7 of the enclosure M5 should be between about 1 cm and about 7 cm and preferably between about 2 cm and 4 cm. The enclosure M5 should also be ergonomically shaped for easy use and handling by the user. The device can be designed as a hand held instrument. In this case the power source M10 may be inside the enclosure or it may external to it. If the energy source M10 is electrical energy source such as a power supply or power outlet or wall plug, electrical wire may be used to bring the energy into the enclosure M5. If the energy source is an compact electrical source such as a battery it may be placed inside the enclosure M5.

FIG. 26 illustrates yet another embodiment for treating skin conditions including acne. Here a control board M40 allows the user to willfully determine the duration and amount of energy delivered to the treating head. The duration of the energy delivery time is generally designed to be between about 0.001 millisecond and about 15 seconds and preferably between about 0.1 millisecond and about 0.5 second. The amount of energy supplied by the energy source should be sufficient to raise the surface temperature between about 39° C. and about 400° C. and preferably between about 50° C. and about 300° C. FIG. 26 also shows a power control button M 60 that can be switched between the off position and different power levels, for example, low, medium and high power level. FIG. 26 also shows a fire button M50 that allows triggering of the circuit board that in turn triggers the release of energy from the energy source to the treatment head.

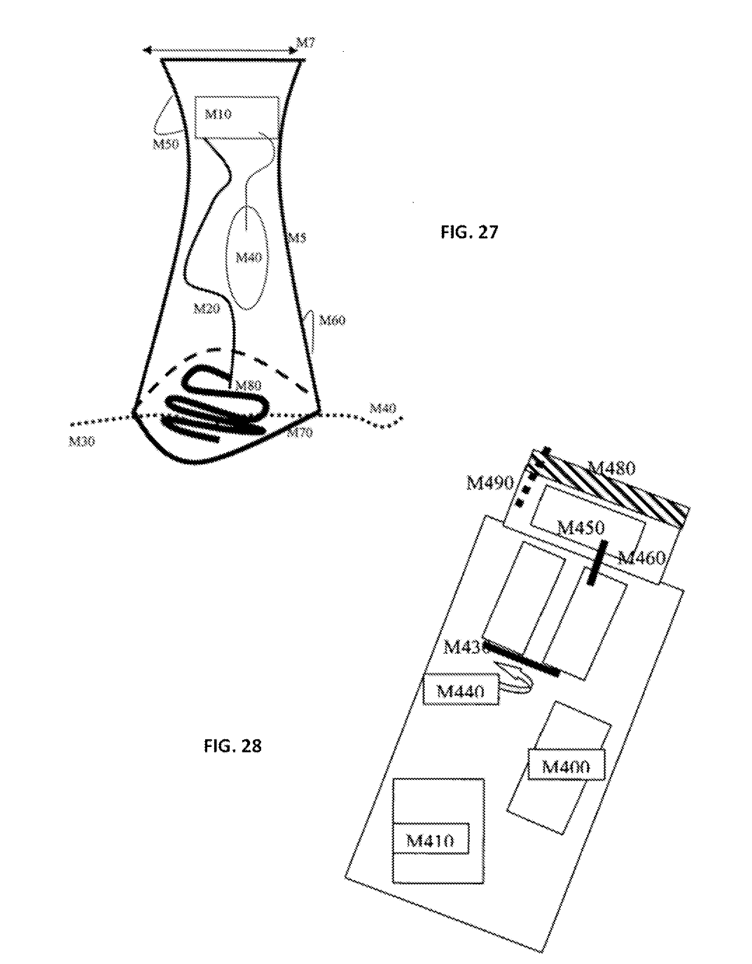

FIG. 27 shows an embodiment wherein an electric source energy M10 delivered a pre-determined amount of energy through an electric current via a wire M20 to a resistive heating element M80 or a thermoelectric cooler M80 designed to heat and or cool (by switching polarity), placed at a footplate M70 which is in contact with the target tissue and preferably skin surface. Preferably the amount of energy delivered to the skin is sufficient to cause skin expansion so that skin pores expand and allow enhanced material transport across the surface, or sterilize bacteria or unwanted organisms within the tissue, or both.

Such electrical energy source should supply energy that should be sufficient to raise the surface temperature to between about 39° C. and about 400° C. and preferably between about 50° C. and about 300° C. for time duration between about microsecond and 100 seconds and preferably between 1 millisecond and 2 seconds.

FIG. 28 shows yet another embodiment designed to minimize charge time of the plurality of lamps. Here a plurality of batteries M410 charge a polarity of capacitors M420, mounted on a rotating plate M430. When the capacitors are rotated in the direction of the arrow M440, a different capacitor is brought into electronic connection with the flash lamp M450 via the electrical contact M460. The electronic board M400 controls the process of charging and rotating the plate M430. An optional absorbing plate M480 can be brought in as an intermediate media that converts the light energy into heat and brought into contact with the skin surface. This can be accomplished, for example, by swinging the absorbing plate on an axis M490 in and out of the lamp light pass.

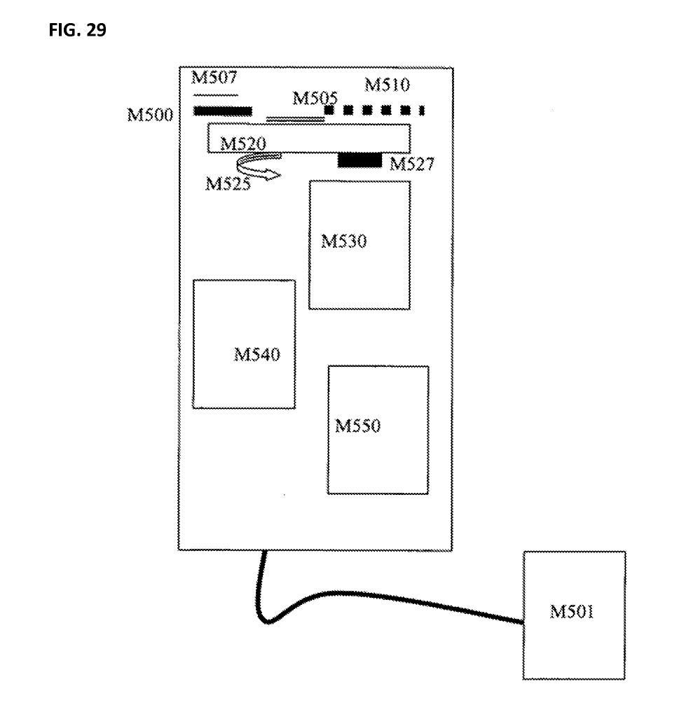

In yet another embodiment, FIG. 29 shows the device wherein, on a rotating or stationary plate M520, a plurality of treatment heads are positioned. The treatment head can be made of a flash lamp, for example a xenon flash lamp M500 with an optional absorbing or partially absorbing interacting layer M507 placed in front of said window. An electrical heater window M505 with an electric resistor for heating or cooling the surface. In this case, an exemplary thermoelectric cooler can be used for example to heat or cool the target surface to a desired temperature in order to open pores or sterilize. Additional, rapid electric heater M510 can be used with an electric pulse sufficient to heat the surface of the target skin to a desired temperature, for a desired length of time. (For example, heating to a temperature range between 40° C. and 350° C. and preferably between about 200° C. and about 330° C., for duration of from about 0.1 ms to about 10 seconds and preferably between 1 ms and 250 ms). Yet another embodiment contemplates additional treating head made of abrasive material, carrying chemical solutions, or delivering vacuum suction or a stream of abrasive particles.

These plurality of treatment heads can be, for example, mounted circularly on the rotating plate M520 and be rotated to deliver a treatment to the targeted surface in sequence or simultaneously. The rotation of the plate is indicated by the arrow M525. Such, heating, (by electrical or optical means) abrasive action, applications of chemicals, and vacuum suction are directed towards opening skin pores and opening, mitigating undesirable skin conditions and skin diseases, enhancing trans-dermal transport, and also reducing longer term skin pore sizes and enhancing the appearance of the skin. Again, the lamps, resistors, or thermo-electric coolers may be powered by capacitors M530 and an energy source M550 or an external energy source M501, and are controlled by an electronic control board M540. The electric heater, Thermo-electric cooler, rotating motor M527 and vacuum sources may be powered by a non-pulsing electric energy source M550 or M501.

The Following Embodiments are a Method for Treating a Target Surface:

The method comprises the steps of a) activating a an energy source, b) bringing an energy transporter element into contact with said heat source, c) allowing said energy transporter element to absorb some of the energy from the heat source, d) disconnecting said energy transporter and moving it into contact with target surface, e) allowing a predetermined amount energy from said energy transporter to be transferred to a target surface so that a desired effect is achieved, wherein the desired effect is a physical, chemical or biological effect, or a thermal change in the target surface characteristics, or a thermal expansion of the target surface, or a thermal expansion of the skin allowing opening of the skin pores so that said expansion allows at least some enhancement of material transport through said skin pores.

A device for thermal material conditioning comprises: a heat source elevated to the desired temperature and maintained at said desired temperature; a heat shuttle in contact with the heat source so that thermal energy can diffuse from the heat source and maintain said heat shuttle at the same temperature as the heat source; a trigger that allow an operator to willfully released from contact with the heat source and is delivered and brought into contact with the target treatment area so that thermal energy can flow from the heat shuttle to the targeted treatment material; allowing said heat shuttle to maintain contact with the targeted treatment area of the target material for a period of time sufficient to bring the target material and the heat shuttle into thermal equilibrium so that substantially no heat flow from the heat shuttle to the targeted material; and removing the heat shuttle from contact with the target surface and bringing it back into a contact with the heat source.

In the method the heat shuttle is allowed to maintain contact with the targeted material area for a period of time from about 0.1 microsecond to about 1 second. The method further comprises bringing the target material surface to a temperature of between about 45 degrees Celsius and 500 degrees Celsius. The method further comprises using the human skin as a target material. The method further comprises bringing the target material surface to a temperature that results in expansion of the skin surface. The method further comprises bringing the target material surface to a temperature that results in effective increase of pore size by at least about 1 micrometer in diameter. The method further comprises repeating all steps at a repetition rate of between about 0.1 Hz and about 1 KHz, and preferably between 0.2 Hz and 10 Hz. In the method the heat source is electrical source of energy

In the method the heat source is a thermo-electric cooler. In the method the heat shuttle is made of metal, such as a thin metal sheet of between about 1 micrometer in thickness and about 10 mm in thickness and preferably between about 70 micrometer and 200 micrometer. The target material is skin.

A device for skin conditioning comprises: a heat source; a heat shuttle in contact with said heat source; a console to contain both the heat source and the Heat shuttle; a transfer compartment capable of separating the heat shuttle from the heat source, and transferring it into contact with the target material, keeping the heat shuttle in contact with said target material for a predetermined period of time, and then removing the heat shuttle from the target material and transferring it back into contact with the heat source.

A device is capable of repeatedly and automatically heating a target material by bringing a Heat Shuttle into high temperature through by keeping the heat shuttle in contact with a heat source, moving the heat shuttle away from the heat source and into contact with a target material to be heated, maintaining contact between the heat shuttle and the target material for a predetermined length of time, removing the heat shuttle from the target material and bringing it back into contact with the heat source and repeating said steps for a predetermined period of time or a predetermined number of repetitions.

In the embodiments, the heat shuttle is kept in contact with the skin target material for a sufficiently long time to allow expansion of the skin so that at least one skin pore expands and opens enough to allow enhanced material transport through said at least one skin pore.

A device for treating material conditions comprises: a heat source, a heat shuttle in contact with said heat source, said heat shuttle comprises a body capable of loading up evenly with thermal energy and two latches.

One latch is connected to a spring which tend to propels the heat shuttle towards the target material and keeps it in contact with said target material.

The second heat latch is picked up (hooked to) by a rotating motor which propels the heat shuttle back up and brings it back into contact with the heat source.

The latch is constructed with a slop so that the rotating motor eventually slips off it allowing the now compressed spring in constant contact with latch number one to propel the heat shuttle again into the target material.

The process is repeated until the operator stops.

In this embodiment the role of the spring and the motor is reversed, i.e. the motor is the one pushing the heat shuttle into the target material and the spring tends to drive the heat shuttle away from the target material and into contact with the heat shuttle.

A device for material conditioning comprises: a magazine full of spring loaded individual heat shuttles (much as in an automatic machine gun magazine), said heat shuttle bullets comprise of at least thin aluminum floor to be loaded with heat energy and two latches, a spring pushing against one latch in order to allow it to create a good thermal contact with the heat source, a motor driving against the other latch to push the heat shuttle down away from the heat source and into contact with the target material, a remover arm pushing the spent heat shuttles (whose thermal energy was used) away from the device and disposing of them), a loader arm pushing the “bullets’ heat shuttles into place where they can be picked up by the spring loading mechanism and be pushed into contact with the heat source.

A motor is used to drive a piston up against a spring (spring loading mechanism). The spring discharge after a stop at the station that allows it to load up with thermal energy. The shuttle is thus propelled by the spring towards the target material to be treated.

The amount of heat energy that was loaded up into the shuttle is finite, so the amount of heat or thermal energy that is discharged into the target material is finite as well.

Acne Contact Device for Home Use and Thermal Skin Conditioning for Home Use

Additional Embodiment are as Follows:

A method for Material Conditioning comprising: a) A heat source brought to a desired temperature and maintained at that temperature; b) A Heat Shuttle (HS) maintained at the source temperature through thermal contact with the Heat Source; c) Means to willfully trigger said heat shuttle (HS) motion so it is released from thermal contact with said heat source and is brought into thermal contact with the targeted treatment area; d) Allowing said heat shuttle to maintain contact with the treatment area for a period of time sufficiently long to transfer sufficient thermal energy to the targeted region to cause thermal expansion of the treated area and bring about the desired effects including the treatment of skin conditions; e) Removing the HS from contact with the targeted area and bringing it back into thermal contact with the heat source

In the method the period of contact between the heat shuttle and the treatment area is from about 0.1 ms to about 1 second and preferably from about 1 ms to about 100 ms (In water-like material such a period of 100 ms will allow thermal energy to diffuse to roughly a depth of penetration of about 300 um).

The method further comprises repeating all steps at the repetition rate of between 0.1 Hz and 1 KHz and preferably at a repletion rate of between 0.2 Hz and 10 Hz.

In the method the heat source is powered by electrical heater driven by electrical energy. In the method the heat source is a thermo-electric cooling device (TEC) or Paltrier cooling device.

In the method the heat shuttle is made of metal of sufficient contact area with the target material to allow reasonable work rate and preferably a contact area with the target material of between about 0.2 cm2 and 4 cm2. In the method the heat shuttle is made of metal of sufficient volume and heat capacity to allow the heat shuttle to carry thermal energy sufficient to raise the temperature of the upper layers of the skin to cause the desired effect and in particular to improve or cure undesired skin conditions. In the method the Heat Shuttle (HS) is made of thermally conducting material in the form of a sheet with a thickness of between about one micrometer and about one millimeter in thickness and preferably between 70 micrometer and 200 micrometer.

A device for material conditioning comprises: a heat source, a heat shuttle in contact with said heat source, a console to contain both the heat source and the heat shuttle (HS) and to ensure that neither is in thermal contact with the target treatment area during at least part of the device operation time, a transfer element capable of separating the heat shuttle from the heat source and bringing it into contact with the target material keeping the heat shuttle, keeping the heat shuttle in contact with said target material for a predetermined period of time then removing the HS from the targeted material and bringing the HS back into thermal contact with the heat source. The device further comprises keeping the HS in contact with the target material for a sufficiently long time to allow thermal expansion of the target material.

The device further comprises a pump to lower the pressure within the device chamber and create a tighter seal to the skin. This will allow: better contact with the skin, removal of debris from the skin and pores, and reduction of the amount of air within the chamber in order to minimize heat conduction and heat removal from the HS during it passage from the heat source to the targeted skin.

In the devices, the heat shuttle can be coated with drug or any other substance that is desirable to deliver into the target surface. A drug or any other substance can be applied to the same area of the skin before, during, or after the action of the heat shuttle.

In the device, a container and dispenser containing and dispensing a drug or any other substance that one wishes to deliver into the target surface is attached to the heat shuttle apparatus and delivers a desirable substance before, during or after the action and passage of the heat shuttle.

A therapeutic treatment device comprises: an incoherent electromagnetic energy source operable to provide a pulsed energy output from a plurality of energy sources having a spectrum of frequencies including a frequency bandwidth capable of being absorbed by an intermediate substance; a housing with an opening, said light source being disposed in said housing, and said housing being suitable for being disposed adjacent to the intermediate substance; a variable pulse-width pulse forming circuit electrically connected to said light source; a reflector mounted within said housing and proximate said light source, directing its energy towards said absorbing intermediate substance whose absorbing characteristics range from zero (completely transmitting) to infinity (completely absorbing), wherein the fluence is less than 2 J/cm2, preferably less than 1 J/cm2. The incoherent energy source is supplemented with a laser energy directed at the general vicinity of the treatment area before, during or after the application of the pulsed energy output. Substantially most of the energy of the electromagnetic source is deposited at the surface resulting in expansion of skin surface opening and discontinuities to allow at least some enhancement in the transport of material across the skin to alleviate skin conditions and ailment and to improve the look and condition of the skin.

The plurality of energy sources can be lamps with reflectors with electromagnetic energy output and at least one lamp energy is intercepted by a high absorbing film mounted proximate to the lamp opening. Said energy source can be a light source, or a flash lamp such as of the type used in digital and disposable (single use) cameras. Said energy source comprises means for providing pulses having a width in the range of between about 0.5 microseconds and 500 millisecond and an energy density of the light on the skin of more than about 0.1 J/cm2. and less than about 2 J/cm2.

Said energy source comprises means for providing a pulse in the range of about 0.1 milliseconds to 2000 milliseconds, whereby skin opening may be expended to enhance transport across the skin.

Said energy source comprises means for providing pulsed electromagnetic energy in the range of about 0.1 millisecond and about 1000 milliseconds, and providing laser CW light radiation before, during, or after said pulse radiation. Said energy source comprises means for providing pulsed electromagnetic energy in the range of about 0.1 millisecond and about 1000 milliseconds, and providing laser CW light radiation before, during, or after said pulse radiation and providing lamp radiation before, during, after, and is able to heat the dermis/epidermis junction temperature to between about 45 degree C. and 55 degree C. Said energy source comprises means for providing pulsed electromagnetic energy in the range of about 0.1 millisecond and about 1000 milliseconds, and providing lamp radiation before, during, after, and is able to heat the dermis/epidermis junction temperature to between about 45 degree C. and 55 degree C.

Said energy source comprises means for providing pulsed electromagnetic energy in the range of about 0.1 millisecond and about 1000 milliseconds, and providing lamp radiation before, during, after, and is able to heat the dermis/epidermis junction temperature so that combined with the energy deposited in the skin by pulse EM energy source, skin conditions are alleviated including the condition of acne.

Said light source comprises means for providing pulses having a width in the range of between substantially 0.05 microsecond and 1000 millisecond and an energy density of the light on the skin of less than about 10 J/cm2.

Said light source comprises means for providing pulses having a width in the range of between substantially 0.1 millisec and 600 millisec and an energy density of the light on the skin of less than about 6 J/cm2.

Said light source comprises means for providing plurality of pulses having a width in the range of between substantially 0.1 millisec and 600 millisec and an energy density of the light on the skin of more than 2.5 J/cm2.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A device for treating skin by delivering a controlled amount of thermal energy to the skin comprising:
a flash lamp for emitting optical energy;
a reflector for directing the optical energy emitted from the flash lamp;
a circuit to deliver a predetermined amount of energy to the flash lamp;
an absorbing layer being placed between the flash lamp and the skin and capable of absorbing at least some of the optical energy discharged by the flash lamp and converting it into thermal energy.

2. The device of claim 1, wherein the absorbing layer comprises a pattern of regions with higher and lower transmissions.

3. The device of claim 1, wherein the absorbing layer is supported on a transparent substrate.

4. The device of claim 1, wherein the absorbing layer is made of absorbing metal

5. The device of claim 1 wherein the absorbing layer is made of absorbing insulator.

6. The device of claim 1, further comprising a power source electrically coupled to a capacitor of the circuit, wherein the capacitor is charged by the power source and discharged by a user-operated external trigger.

7. A device for treating skin, the device comprising:
a treatment head coupled to a housing;
a window positioned on the treatment head;
a light source for transmitting a predetermined amount of energy out of the window to the skin, wherein the predetermined amount of energy is between about 0.1 J/cm2 to about 20 J/cm2;
a plurality of protruding guards, the protruding guards extending outward from the treatment head in a direction substantially perpendicular to the surface of the window, the protruding guards comprising a proximal portion secured to the treatment head and a distal portion extending away from the treatment head, wherein the protruding guards are positioned on and extending from the window, wherein the protruding guards comprise light absorbing material so that as the protruding guards are being pressed against the skin it also absorbs energy generated by the light source; and
an electronic safety circuit having an open configuration wherein activation of the light source is prevented, and further having a closed configured wherein activation of the light source is permitted, the electronic safety circuit being biased toward the open configuration, the electronic safety circuit being movable to the closed configuration responsive to pressure being applied to any of the protruding guards distal portion toward the protruding guards proximal portion.

8. The device of claim 7, further comprising:
a hair remover configured to remove hair from the target area of skin, the hair remover mounted on the treatment head, wherein the hair remover and the window are mounted on the treatment head; and
a first cleaner for cleaning the target area of the skin treated with the hair remover, wherein the first cleaner is mounted on the treatment head at a position between the hair remover and the light source.

9. The device of claim 7, wherein the hair remover comprises a blade or an adhesive tape.

10. The device of claim 7, wherein the light source comprises a flash lamp or a LED.

11. The device of claim 7, wherein two or more protruding guards are positioned around the window and the electronic safety circuit is movable to the closed configuration only when all protruding guards are simultaneously depressed.

12. A method to treat injuries, comprising:
a. Providing an energy source
b. Providing a controller
c. Adjusting the energy source parameters
d. Providing a delivery member
e. Providing A coupling member
f. Treating the targeted injured region of a tissue by delivering energy from said energy source.

13. The method of claim 12, wherein the energy source is at least one of: Laser LED, Flash lamp, Ultrasound, RF source, Electromagnetic energy source, Mechanical energy source, Electrical energy source, Magnetic energy source, X ray energy source, Chemical energy source.

14. The method of claim 12, wherein said controller include at least from the group of: Microprocessor, Laptop computer, Hardwire controller, Interactive microprocessor.

15. The method of claim 12 wherein said parameters of the energy source to be adjusted include at least one of: Duration of the energy output, Repetition rate of the energy coming out of the energy source, Total energy emitted by the energy source, Spot size of the energy at the tissue surface, Type of energy coming out of the energy source, Wavelength of the energy coming out of the energy source, Divergence or convergence of the energy from the energy source at the target surface, Location of the focus of the beam of energy emerging from the energy source.

16. The method of claim 12, wherein said delivery member is at least one of a group including: Optical fiber, Lens, Window, Window with protruding member, A mechanical compressor, A vibrating mechanical oscillator, A hummer head, A compression head with opening or light or fiber optics, A compression head with opening for cooling or heating member, A compression head with suction, A window with suction.

17. The method of claim 12, wherein the coupling member comprises one or more of the following members: A window, A lens, A plurality of micro lenses, A protruding guard, A protruding member.

18. The method of claim 12, wherein the method further comprises the step of providing and imaging or feedback member.

19. The method of claim 18, wherein said imaging or feedback member comprises one or more of the following: An ultrasound imaging system, An Optical Coherence Tomography device, A microscope, A camera, An optical imager, A LIBS analyzer, A fluorescence detection, A second harmonic generation or other nonlinear imager, CAD, or x ray imaging.

20. The method of claim 12, wherein treatment step comprises allowing the energy from the energy source to reach the targeted surface and penetrate said targeted surface.
